(19) United States
(12) Patent Application Publication
(10) Pub. No.: US 2005/0008649 A1
(43) Pub. Date: Jan. 13, 2005

(54) Chimeric molecules and methods of use

(75) Inventors: Seung-Uon Shin, Miami, FL (US); Sherie L. Morrison, Los Angeles, CA (US); Joseph D. Rosenblatt, Hollywood, FL (US)

Correspondence Address: AKERMAN SENTERFITT, P.O. BOX 3188, WEST PALM BEACH, FL, 33402-3188, US

(73) Assignee: University of Miami, Miami, FL (US)

(21) Appl. No.: 10/858,980

(22) Filed: Jun. 2, 2004

Related U.S. Application Data
(60) Provisional application No. 60/475,015, filed on Jun. 2, 2003.

(51) Int. Cl. A61K 39/395; A61K 39/00; C07K 16/44

(52) U.S. Cl. 424/178.1; 424/192.1; 530/387.3

(57) ABSTRACT

Chimeric molecules comprising endostatin and all or a portion of an Ig (Ig) molecule are used to treat tumors. A chimeric molecule, including endostatin fused to an Ig domain of an anti-HER2/neu antibody exhibited longer serum half-life and stability than native endostatin. ¹²⁵I-labeled anti-HER2/neu IgG3-endostatin chimeric molecule and anti-HER2/neu IgG3 preferentially localized to CT26-HER2 tumors. Clearance of anti-HER2/neu IgG3-endostatin was 6 fold faster than that of anti-HER2/neu IgG3 (CLss=0.374 and 0.062 ml/min/kg, respectively), however, the specific tumor radiolocalization indices of anti-HER2/neu IgG3-endostatin were greater than those of anti-HER2/neu IgG3. Anti-HER2/neu IgG3-endostatin inhibited tumor growth more effectively than endostatin alone, anti-HER2/neu IgG3 antibody, or the combination of antibody and endostatin.

### FIELD OF THE INVENTION

The invention relates to compositions and methods for targeting and modulating the activity of tumor cells. In particular, the invention relates to chimeric fusion molecules which have a tumor antigen targeting domain and an effector function domain. Furthermore, the chimeric fusion molecules have a greater serum half-life than either of the native parent molecules alone.

### BACKGROUND OF THE INVENTION

Anti-angiogenic tumor therapies have recently attracted intense interest because of their broad-spectrum action, low toxicity, and absence of drug resistance. Endostatin is a recently characterized anti-angiogenic agent. Although the mechanism of action of endostatin is not clear yet, the anti-tumor activity of endostatin may be associated with inhibiting the proliferation and migration of endothelial cells. In addition, endostatin may down-regulate VEGF expression in tumor cells.

A number of animal experiments and human clinical trials have been performed to assess the anti-tumor effect of endostatin. Systemic administration of endostatin at 10 mg/kg suppressed the growth of human renal cell cancer in a nude mouse xenograft model. In early human phase I trials, endostatin administration at high dose levels (240 mg/m²/day) in the range of active levels established in tumor xenograft studies did not show any significant detectable changes in biologic endpoints, such as urinary excretion levels of VEGF and basic FGF. However, modest clinical benefit was observed in three out of 15 patients. One patient with a pancreatic neuroendocrine tumor had a minor tumor reduction, and disease in two other patients briefly stabilized. Another human phase I trial demonstrated that endostatin was well tolerated and did not induce dose-limiting toxicity at dose-levels up to 600 mg/m²/day, but little anti-tumor activity was seen in 25 patients, even at circulating levels beyond those previously noted to be effective in mouse models. Two patients (one with sarcoma, one with melanoma) demonstrated minor and short-lived anti-tumor activity. The first two phase I clinical trials proved that endostatin is a very safe drug in a variety of dose schedules. However these results did not demonstrate substantial endostatin anti-tumor activity. The dose and schedules may have been suboptimal, and/or bulky disease in late stage patients may not be optimally responsive to recombinant human endostatin. Anti-angiogenic therapy in cancer patients may therefore require prolonged administration of recombinant protein.

Many anti-angiogenic agents, however, are unstable in vitro and in vivo. Endostatin has a short half-life in mice (T_{1/2}2=38-225 min) and only 55% of circulating endostatin is TCA precipitable at one hour. The short in vivo half-lives, and the serum instability of endostatin currently necessitate its administration by frequent or continuous injection. The instability of endostatin may minimize its clinical efficacy. While one could improve results by using continuous dosing or higher dosage levels, a theoretical risk exists that persistent, uncontrolled non-specific anti-angiogenic therapy might have deleterious side effects on normal physiologic processes such as endometrial maturation and corpus luteum formation, embryo growth, the angiogenic response to chronic ischemia in the heart and lower limbs, wound healing, and hair growth. In early trials, the nonspecific inhibition of angiogenesis using high levels of an anti-VEGF antibody (AVASTIN™) has resulted in life threatening pulmonary hemorrhage in a subset of patients.

Anti-angiogenic gene therapy has been proposed as an alternative way to continuously provide high concentrations of the anti-angiogenic factors. Gene transfection of anti-angiogenic agents using a viral vector can inhibit the growth of tumor in several mouse models. Viral vectors, however, may cause inflammation and immunological response on repeated injection, and toxicity/safety considerations may preclude their use in humans in the near future. Furthermore, use of gene-transduced hematopoietic stem cells has been ineffective in an animal model, despite sustained production of endostatin.

Several logistical disadvantages of the long-term treatment with high dosages of endostatin may be overcome if the half-life of endostatin could be extended and if endostatin could be specifically targeted to the tumor, to achieve higher local concentrations and greater specificity.

### SUMMARY

The invention relates to the development of tumor-targeting chimeric molecules comprising both (1) an anti-angiogenic agent and (2) a carrier domain such as all or a portion of an immunoglobulin (Ig) molecule. In the illustrative embodiments described below, an anti-angiogenic agent-Ig chimeric molecule that includes an Ig domain from an anti-HER2/neu antibody fused to endostatin to form anti-HER2/neu IgG3-endostatin. The latter exhibited longer serum half-life and stability than did native endostatin. In mice implanted with CT26 and CT26 expressing HER2/neu (CT26-HER2) tumors on opposite flanks, ¹²⁵I-labeled anti-HER2/neu IgG3-endostatin chimeric molecule and anti-HER2/neu IgG3 preferentially localized to CT26-HER2 tumors. The clearance of anti-HER2/neu IgG3-endostatin was 6 fold faster than that of anti-HER2/neu IgG3 (CLss=0.374 and 0.062 ml/min/kg, respectively). However, the specific tumor radiolocalization indices of anti-HER2/neu IgG3-endostatin were greater than those of anti-HER2/neu IgG3.

Equimolar administration of anti-HER2/neu IgG3-endostatin to mice bearing both CT26 and CT26-HER2 showed preferential inhibition of CT26-HER2, compared to CT26 parental tumor contralaterally implanted within the same mice. Anti-HER2/neu IgG3-endostatin inhibited more effectively than endostatin, anti-HER2/neu IgG3 antibody, or the combination of antibody and endostatin. The longer half-life and serum stability of the anti-HER2/neu IgG3-endostatin chimeric molecule coupled with ability to selectively target antigens expressed on tumors results in increased suppression of angiogenesis.

In a preferred embodiment, the invention provides a pharmaceutical composition comprising a chimeric fusion molecule, wherein the chimeric fusion molecule comprises an antigen binding domain and a therapeutic effector domain. Preferably, the pharmaceutical composition is used in treating cancer.

In another preferred embodiment, the antigen binding domain comprises an isolated antibody or fragments thereof. The isolated antibody or fragments thereof comprises immunoglobulin heavy and light chains and/or immunoglobulin variable and constant regions. Preferably, the isolated immunoglobulin variable region comprise Fab, Fab′, F(ab′)₂, and Fv fragments and/or immunoglobulin constant regions, C_{H}1, hinge, C_{H}2 and C_{H}3.

In another preferred embodiment, the isolated antibody or fragments thereof are fused to a therapeutic effector domain. In accordance with the invention, the isolated antibody is fused to the therapeutic effector domain via the immunoglobulin constant regions, C_{H}1, hinge, C_{H}2 or C_{H}3. Preferably, the isolated antibody is fused to the therapeutic effector domain via the immunoglobulin constant region, C_{H}3.

In one embodiment, the therapeutic effector domain comprises a molecule for modulating cellular activity or is cytolytic. Preferably, the therapeutic effector domain's cellular modulating activity inhibits angiogenesis. Also preferred, is that the therapeutic effector domain's cellular modulating activity modulates immune cell responses.

In one preferred embodiment, the therapeutic effector domain is endostatin, angiostatin, basement-membrane collagen-derived anti-angiogenic factors tumstatin, canstatin, or arrestin.

In another preferred embodiment, the therapeutic effector domain comprises chemokines, cytolytic molecules and/or interferon. In accordance with the invention, the cytolytic molecule is TNF and/or toxin.

In another preferred embodiment, the antibody domain binds to a tumor antigen. The tumor antigen is preferably, HER2/neu.

In another preferred embodiment, the invention provides for an isolated nucleic acid molecule encoding the chimeric molecule as described infra and nucleic acid molecules encoding the chimeric molecule.

In another preferred embodiment, the invention provides a chimeric fusion protein comprising a tumor specific antibody or fragment thereof fused to an anti-angiogenic agent. In accordance with the invention, the tumor specific antibody binds to HER2/neu and the anti-angiogenic agent is endostatin, angiostatin, basement-membrane collagen-derived anti-angiogenic factors tumstatin, canstatin, or arrestin.

In another preferred embodiment, the antibody or fragment thereof is IgG3. preferably, the IgG3 constant region (C_{H}3) is fused to endostatin.

In another preferred embodiment, the chimeric fusion protein is administered to a patient in need of such therapy and modulates the activity of the tumor.

In another preferred embodiment, the serum half-life of the chimeric fusion protein is at least about 50% greater than the half-life of the anti-HER2/neu antibody, preferably, the serum half-life of the chimeric fusion protein is at least about 80% greater than the half-life of the anti-HER2/neu antibody, preferably, the serum half-life of the chimeric fusion protein is at least about 100% greater than the half-life of the anti-HER2/neu antibody.

In another preferred embodiment, the serum half-life of the chimeric fusion protein is at least about 50% greater than the half-life of endostatin, preferably, the serum half-life of the chimeric fusion protein is at least about 80% greater than the half-life of endostatin, preferably, the serum half-life of the chimeric fusion protein is at least about 100% greater than the half-life of endostatin.

In another preferred embodiment, the chimeric fusion protein inhibits angiogenesis by at least about 10% as compared to an untreated individual, preferably, the chimeric fusion protein inhibits angiogenesis by at least about 50% as compared to an untreated individual, preferably, the chimeric fusion protein inhibits angiogenesis up to 100% as compared to an untreated individual.

In another preferred embodiment, the invention provides a method for targeting endostatin to a tumor cell in an animal subject, the method comprising the step of administering to the animal subject a composition comprising a chimeric molecule comprising an endostatin domain and an Ig domain.

In another preferred embodiment, the invention provides a method for treating a tumor in an animal subject, the method comprising the step of administering to the animal subject a composition comprising a chimeric fusion molecule composition, as described above. Preferably, the chimeric fusion molecule composition is administered with one or more therapeutic agents and/or adjuvants.

In other preferred embodiments, the therapeutic agents comprise antiangiogenic antibodies, tumor antigen specific antibodies, glycolysis inhibitor agents, anti-angiogenic agents, chemotherapeutic agents, radiotherapy, radionuclides, or drugs that ameliorate the symptoms of a patient.

In accordance with the invention, the chimeric fusion molecule composition is administered to a patient in combination with metronomic therapy. For example, administration of continuous low-doses of the chimeric fusion molecule and one or more therapeutic agents. Therapeutic agents can include, for example, chemotherapeutic agents such as, cyclophosphamide (CTX, 25 mg/kg/day, p.o.), taxanes (paclitaxel or docetaxel), busulfan, cisplatin, cyclophosphamide, methotrexate, daunorubicin, doxorubicin, melphalan, cladribine, vincristine, vinblastine, and chlorambucil.

In another preferred embodiment, the invention provides a kit comprising, a chimeric molecule comprising a domain targeting the chimeric molecule to HER2/neu tumor antigen and a domain comprising an anti-angiogenic agent. Preferably, the domain comprising the anti-angiogenic agent is endostatin of fragments thereof. Also preferred is a domain targeting the chimeric molecule to HER2/neu tumor antigen is an antibody or fragments thereof.

In accordance with the invention the antibody or fragments thereof is preferably, polyclonal or monoclonal. Further provided is a pharmaceutical composition for administering the chimeric molecule to a patient in need thereof. The chimeric fusion molecule may be lyophilized and reagents and/or pharmaceutical compositions for reconstituting and administering the lyophilized chimeric molecule are provided.

Additionally, instructions for carrying out the method for administering the chimeric molecule to a patient, are provided.

Unless otherwise defined, all technical terms used herein have the same meaning as commonly understood by one of ordinary skill in the art to which this invention belongs. Commonly understood definitions of molecular biology terms can be found in Rieger et al., Glossary of Genetics: Classical and Molecular, 5th edition, Springer-Verlag: New York, 1991; and Lewin, Genes V, Oxford University Press: New York, 1994.

Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions will control. In addition, the particular embodiments discussed below are illustrative only and not intended to be limiting.

Other aspects of the invention are described infra.

### BRIEF DESCRIPTION OF THE DRAWINGS

FIG. 1 is a schematic illustration of various anti-HER2/neu IgG3-endostatin fusion proteins within the invention.

FIG. 2A-C shows the results of serum clearance and stability in mice bearing CT26-HER2/neu tumors. Serum clearance (A) and serum TCA precipitability (B) of [1251] labeled anti-HER2/neu IgG3-C_{H}3-Endo (filled square), anti-dansyl IgG3 (open circle), anti-HER1/neu IgG3 (filled circle) and endostatin (open square) were measured. Measurements of anti-HER/neu IgG3 and anti-HER/neu IgG3-C_{H}3-Endo were made 96 hours after intravenous injection and those of endostatin 60 min. Data are mean±S.E. (n=3, BALB/c mice). Serum samples of [¹²⁵I] labeled proteins were analyzed by SDS-PAGE (C). At 15 min-1 hr 1.5 ul of anti-HER2/neu IgG3-C_{H}3-Endo, anti-HER2/neu IgG3, and anti-dansyl IgG3 was analyzed while 3 ul of serum was analyzed at 3-96 hr. In case of endostatin, 1.5 ul of serum at 15 sec-i min and 3 ul of serum at 35-60 min were resolved. Each iodinated initial protein (I, 0.35 ul) was used as a control for its own serum samples. [1251] labeled anti-HER2/neu IgG3 was used as a control (II).

FIG. 3A-B shows the targeting of anti-HER2/neu IgG3 (A) and anti-HER2/neu IgG3-C_{H}3-Endo (B) to CT26-HER2/neu tumors, CT-26 tumors, or other organs in a BALB/c mice. Specific tumor targeting is expressed as the percent of the injected dose per gram of tissues.

FIG. 4. Anti-tumor activity of anti-HER2/neu IgG3-C_{H}3-Endo, anti-HER2/neu IgG3 and endostatin. BALB/c mice (n=5 per group) were s.c. injected with CT26-HER2/neu (1×10⁶ cells per mouse), followed on day 7 by equimolar injections every other day (arrow, 5 times) of anti-HER2/neu IgG3-C_{H}3-Endo (20 ug/injection, closed circle), anti-HER2/neu IgG3 (17 ug/injection, open square), anti-dansyl IgG3 (17 ug/injection, open triangle), or endostatin (8 ug/injection, closed square, 40 ug/injection, closed triangle). PBS, phosphate buffered saline (control, open circle). Asterisk marks indicate that the tumor growth following anti-HER2/neu IgG3-C_{H}3-Endo injection is significantly delayed compared to that of PBS (p<0.05, Student t test).

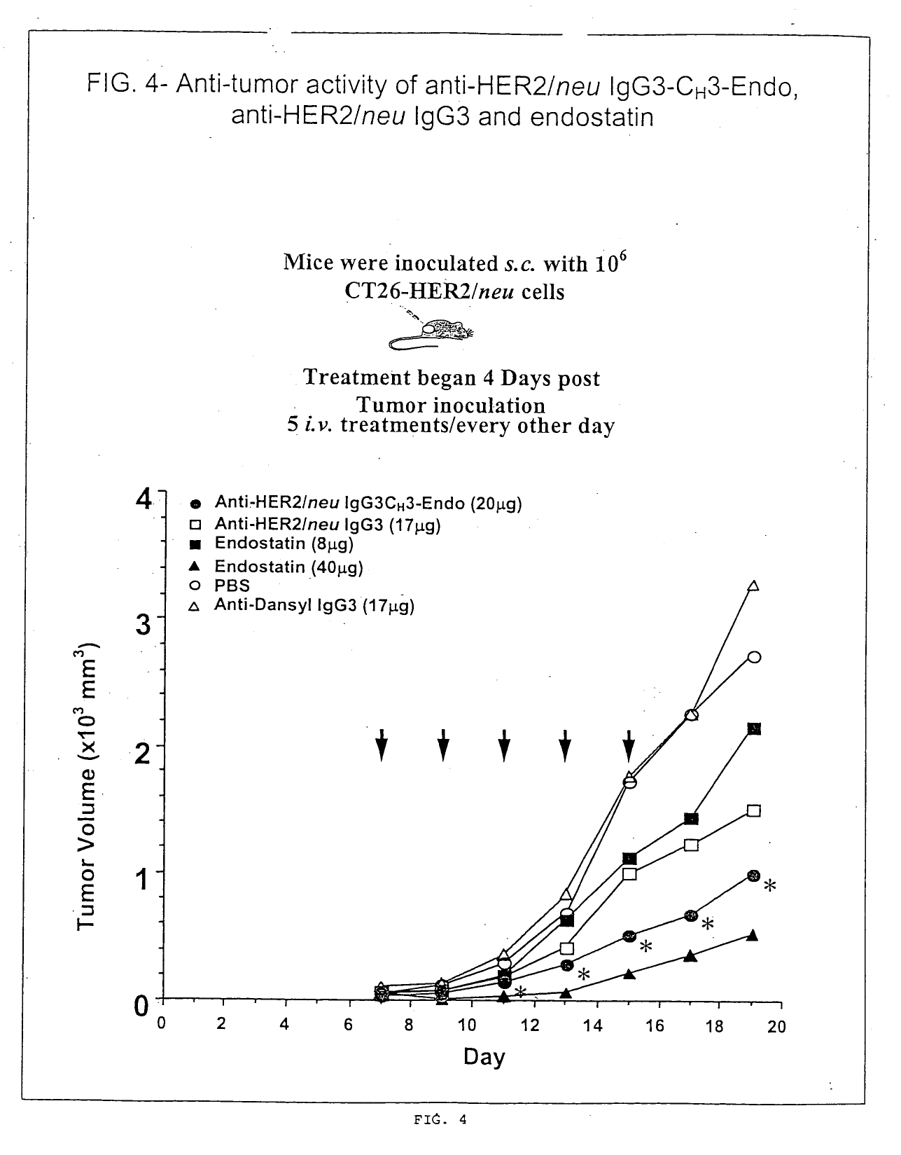

FIG. 5. Anti-tumor activity of anti-HER2/neu IgG3-C_{H}3-Endo, anti-HER2/neu IgG3, endostatin, and combination of anti-HER2/neu IgG3 and endostatin in a BALB/c mice (n=8 per group) bearing both CT26-HER2/neu tumors and contralaterally implanted CT-26 tumors. On day 7, equimolar proteins were injected every other day (arrow, 7 times).

FIG. 6A-D shows a schematic illustration of a chimeric fusion molecule and a SDS-PAGE analysis of anti-HER2/neu IgG3-endostatin fusion protein. A schematic diagram of the secreted H2L2 forms of anti-HER2/neu IgG3-endostatin fusion protein is shown (A). The secreted IgG3-endostatin fusion protein (1) biosynthetically labeled with [³⁵S] methionine was immunoprecipitated with rabbit anti-human IgG and a 10% suspension of staphylococcal protein A, and analyzed under non-reducing (B) and reducing (C) conditions. Endostatin fusion protein purified by protein A affinity chromatography was analyzed under non-reducing conditions (D). Anti-HER2/neu IgG3 (2), anti-dansyl IgG (3), and endostatin (4) are included for comparison.

FIG. 7 is a graph showing inhibition of the angiogenic response mediated by VEGF/bFGF. Purified anti-HER2/neu IgG3-endostatin preparation #1 (open circle) and preparation #2 (closed circle) were added to an aliquot of Vitrogen supplemented with a combination of VEGF and bFGF, and the mixture was placed on a nylon mesh. The impregnated mesh were placed on the chick embryo and incubated. New vessel growth was visualized with fluorescein isothiocyanate dextran and measured by fluorescent intensity. Anti-HER2/neu IgG3 (closed triangle) and endostatin (open triangle) are included for comparison. Positive control group (closed square) contains VEGF/bFGF alone, but negative control group (open square) contains only vehicle. Data are mean±SEM (n=5).

FIG. 8A-C shows serum clearance and stability in mice bearing CT26-HER2 tumors. Serum clearance (A) and serum TCA precipitability (B) of [¹²⁵I] labeled anti-HER2/neu IgG3-endostatin (filled circle), anti-dansyl IgG3 (open square), anti-HER1/neu IgG3 (filled square) and endostatin (open circle) were measured. Measurements of anti-HER/neu IgG3 and anti-HER/neu IgG3-endostatin were made 96 hours after intravenous injection and those of endostatin 60 min. Data are mean±SEM (n=3, BALB/c mice). Serum samples of [¹²⁵I] labeled proteins were analyzed by SDS-PAGE (C). At 15 min-1 hr 1.5 μl of anti-HER2/neu IgG3-endostatin, anti-HER2/neu IgG3, and anti-dansyl IgG3 was analyzed while 3 μl of serum was analyzed at 3-96 hr. In case of endostatin, 1.5 μl of serum at 15 sec-1 min and 3 μl of serum at 5-60 min were resolved. Each iodinated initial protein (Int, 0.35 μl) was used as a control for its own serum samples. [¹²⁵I] labeled anti-HER2/neu IgG3 (IgG3) was used as a control.

FIG. 9A-D shows the targeting of anti-HER2/neu IgG3 and anti-HER2/neu IgG3-endostatin to CT26-HER2 tumors, CT-26 tumors, or other organs in BALB/c mice. A: Two groups of BALB/c mice (12 mice per group) were injected s.c. with 106 single-cell suspensions of either CT26-HER2 (closed histogram) or CT-26 (open histogram). When the tumors were about 5 mm in diameter, [¹²⁵I] labeled proteins (1; anti-dansyl IgG3, 2; anti-HER2/neu IgG3, 3; anti-HER2/neu IgG3-endostatin, 4; endostatin) were injected into four groups of mice (n=3 per protein) through the tail. Specific tumor targeting is expressed as the radiolocalization index (the % ID/g in tumor divided by the % ID/g in blood). B-D: CT26 and CT26-HER2 were contralaterally implanted within the same mice (n=3 per group) and indicated iodinated proteins (C: anti-HER2/neu IgG3, D: anti-HER2/neu IgG3-endostatin) injected. Following injection the indicated tissues were harvested and % ID/g measured as outlined in the Methods. Specific tumor targeting is expressed as the percent of the injected dose per gram of tissues. Data are mean±SEM.

FIG. 10A-C shows the anti-tumor activity of anti-HER2/neu IgG3-endostatin fusion protein in a syngeneic mouse model. BALB/c mice (n=8 per group) were s.c. implanted contralaterally with CT26 and CT26-HER2 (1×10⁶ cells per mouse), followed on day 7 by equimolar injections every other day (arrow, 7 times) of anti-HER2/neu IgG3-endostatin, anti-HER2/neu IgG3, endostatin, and combination of anti-HER2/neu IgG3 and endostatin. Data are mean±SEM.

FIG. 11 is a graph showing anti-tumor activity of anti-HER2/neu IgG3-endostatin in SCID mouse model bearing human breast cancer SK-BR-3. SCID mice were were s.c. implanted with SK-BR-3 (1×10⁶ cells per mouse). On day 15, equimolar proteins of anti-HER2/neu IgG3-endostatin, anti-HER2/neu IgG3, endostatin, and combination of anti-HER2/neu IgG3 and endostatin were injected every other day (arrow, 10 times). Data are mean±SEM.

FIG. 12 shows the immunohistochemical staining of blood vessels in CT26 and CT26-HER2 tumors. Cryosections of CT26 and CT26-HER2 tumors with/without treatments were stained with anti-CD31 antibody or anti-HER2/neu antigen. CT26 tumor: A-C and G-I, CT26-HER2 tumor: D-F and J-L; no treatment (PBS): A-F, treatment with anti-HER2/neu IgG3-endostatin. Images are magnified 100× for A, D, G, and J, and the others were magnified 400×.

FIG. 13A-E shows the analysis of vessel morphology. A-D: Visualization of blood vessel formation in CT26 and CT26-HER2 tumors. Tumor sections were prepared from (A) CT26 and (B) CT26-HER2 tumors without treatments (PBS), or (C) CT26 and (D) CT26-HER2 tumors with treatments of anti-HER2/neu IgG3-endostatin. Each cryosection was stained with rat anti-mouse CD31 and anti-rat IgG-Alexa 594 (red fluorescence). 14-21 digital images of the magnification with 400× were obtained per section, and the above images are composite figures. FIG. 13E: Quantification of blood vessel area in CT26 and CT26-HER2 tumors. The composed images have been analyzed using NIH ImageJ v1.31 by color image to form a binary image to measure blood vessel density. Blood vessel area (pixel2) was then computed. Data are mean±SEM.

### DETAILED DESCRIPTION

The invention provides methods and compositions for targeting a chimeric molecule containing both (1) anti-angiogenic agent and (2) a carrier domain such as all or a portion of an Ig molecule to a tumor. The below described preferred embodiments illustrate adaptations of these compositions and methods. Nonetheless, from the description of these embodiments, other aspects of the invention can be made and/or practiced based on the description provided below.

Definitions

Prior to setting forth the invention, definitions of certain terms which are used in this disclosure are set forth below:

As used herein, the singular forms “a”, “an” and “the” include plural referents unless the context clearly dictates otherwise.

As used herein, “antibody” refers to single chain, two-chain, and multi-chain proteins and glycoproteins belonging to the classes of polyclonal, monoclonal, chimeric, and hetero immunoglobulins (monoclonal antibodies being preferred); it also includes synthetic and genetically engineered variants of these immunoglobulins. “Antibody fragment” includes Fab, Fab′, F(ab′)₂, and Fv fragments, as well as any portion of an antibody having specificity toward a desired target epitope or epitopes.

As used herein, the term “immunoglobulin” refers to a protein consisting of one or more polypeptides substantially encoded by immunoglobulin genes. The recognized immunoglobulin genes include the κ, λ, α, γ (IgG1, IgG2, IgG3, IgG4), δ, ε and μ constant region genes, as well as the myriad immunoglobulin variable region genes. Full-length immunoglobulin “light chains” (about 25 KDa or 214 amino acids) are encoded by a variable region gene at the NH₂-terminus (about 110 amino acids) and a kappa or lambda constant region gene at the COOH—terminus. Full-length immunoglobulin “heavy chains” (about 50 KDa or 446 amino acids), are similarly encoded by a variable region gene (about 116 amino acids) and one of the other aforementioned constant region genes, e.g., gamma (encoding about 330 amino acids). One form of immunoglobulin constitutes the basic structural unit of an antibody. This form is a tetramer and consists of two identical pairs of immunoglobulin chains, each pair having one light and one heavy chain. In each pair, the light and heavy chain variable regions are together responsible for binding to an antigen, and the constant regions are responsible for the antibody effector functions. In addition to antibodies, immunoglobulins may exist in a variety of other forms including, for example, Fv, Fab, and F(ab′)₂, as well as bifunctional hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., “Immunology”, Benjamin, N.Y., 2nd ed. (1984), and Hunkapiller and Hood, Nature, 323, 15-16 (1986), which are incorporated herein by reference).

An immunoglobulin light or heavy chain variable region consists of a “framework” region interrupted by three hypervariable regions, also called CDR's. The extent of the framework region and CDR's have been precisely defined (see, “Sequences of Proteins of Immunological Interest,” E. Kabat et al., U.S. Department of Health and Human Services, (1983); which is incorporated herein by reference). The sequences of the framework regions of different light or heavy chains are relatively conserved within a species. As used herein, a “human framework region” is a framework region that is substantially identical (about 85% or more, usually 90-95% or more) to the framework region of a naturally occurring human immunoglobulin. The framework region of an antibody, that is the combined framework regions of the constituent light and heavy chains, serves to position and align the CDR's. The CDR's are primarily responsible for binding to an epitope of an antigen.

As used herein, “humanized antibody” refers to an antibody derived from a non-human antibody, typically murine, that retains or substantially retains the antigen-binding properties of the parent antibody but which is less immunogenic in humans. This may be achieved by various methods including (a) grafting only the non-human CDRs onto human framework and constant regions with or without retention of critical framework residues, or (b) transplanting the entire non-human variable domains, but “cloaking” them with a human-like section by replacement of surface residues. Such methods as are useful in practicing the present invention include those disclosed in Jones et al., Morrison et al., Proc. Nat'l Acad. Sci. USA, 81:6851-6855 (1984); Morrison and Oi, Adv. Immunol., 44:65-92 (1988); Verhoeyen et al., Science, 239:1534-1536 (1988); Padlan, Mol. Immunol., 28:489-498 (1991); Padlan, Mol. Immunol., 31(3):169-217 (1994).

As used herein, “Complementarity Determining Region” (CDR) refers to amino acid sequences which together define the binding affinity and specificity of the natural Fv region of a native immunoglobulin binding site as delineated by Kabat et al. (1991).

As used herein, “Framework Region” (FR) refers to amino acid sequences interposed between CDRs. These portions of the antibody serve to hold the CDRs in an appropriate orientation for antigen binding. In the antibodies and antibody fragments of the present invention, comprise their fully human native amino acid sequences and/or comprise amino acid sequence modifications necessary to retain or increase binding affinity and/or binding specificity.

As used herein, “constant region” refers to the portion of the antibody molecule which confers effector functions. Preferred constant regions are gamma 1 (IgG1), gamma 3 (IgG3) and gamma 4 (IgG4). More preferred is a constant region of the gamma 3 (IgG3) isotype. The light chain constant region can be of the kappa or lambda type, preferably of the kappa type.

As used herein “chimeric molecule” comprises antibody sequences and a molecule genetically fused to the antibody fragment. For example, a chimeric molecule comprises endostatin genetically fused to an anti-HER2/neu IgG3 heavy chain at the end of C_{H}3, and expressed with an anti-HER2/neu K light chain.

Substantially homologous immunoglobulin sequences are those which exhibit at least about 85% homology, usually at least about 90%, and preferably at least about 95% homology with a reference immunoglobulin protein.

As used herein, “therapeutic effector domain” refers to any molecule that modulates a cellular activity or is cytolytic. For example, a cytokine such as IL-2 modulates T-cell activity; endostatin modulates cellular activity by down-regulating VEGF expression in tumor cells. A modulatory polypeptide or a cytolytic polypeptide is fused to at least one of the first or second polypeptides or the peptide linker. It is preferred that the modulatory polypeptide is anti-angiogenic, such as for example, endostatin. However, the invention is not limited to endostatin. Other examples include, but not limited to, chemokines, angioarrestin, angiostatin (plasminogen fragment), anti-angiogenic antithrombin III, cartilage-derived inhibitor (CDI), CD59 complement fragment, fibronectin fragment, gro-beta, heparinases, heparin hexasaccharide fragment, human chorionic gonadotropin (hCG), interferon alpha/beta/gamma, interferon inducible protein (IP-10), interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-b), vasculostatin, vasostatin (calreticulin fragment).

As used herein, “immunogenicity” refers to a measure of the ability of a targeting protein or therapeutic moiety to elicit an immune response (humoral or cellular) when administered to a recipient. The present invention is concerned with the immunogenicity of the subject humanized antibodies or fragments thereof.

The term “polyclonal” refers to antibodies that are heterogeneous populations of antibody molecules derived from the sera of animals immunized with an antigen or an antigenic functional derivative thereof. For the production of polyclonal antibodies, various host animals may be immunized by injection with the antigen. Various adjuvants may be used to increase the immunological response, depending on the host species.

“Monoclonal antibodies” are substantially homogenous populations of antibodies to a particular antigen. They may be obtained by any technique which provides for the production of antibody molecules by continuous cell lines in culture. Monoclonal antibodies may be obtained by methods known to those skilled in the art. See, for example, Kohler, et al., Nature 256:495-497, 1975, and U.S. Pat. No. 4,376,110.

As used herein, an “antigenic determinant” is the portion of an antigen molecule that determines the specificity of the antigen-antibody reaction. An “epitope” refers to an antigenic determinant of a polypeptide. An epitope can comprise as few as 3 amino acids in a spatial conformation which is unique to the epitope. Generally an epitope consists of at least 6 such amino acids, and more usually at least 8-10 such amino acids. Methods for determining the amino acids which make up an epitope include x-ray crystallography, 2-dimensional nuclear magnetic resonance, and epitope mapping e.g. the Pepscan method described by H. Mario Geysen et al. 1984. Proc. Natl. Acad. Sci. U.S.A. 81:3998-4002; PCT Publication No. WO 84/03564; and PCT Publication No. WO 84/03506.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to marker “X” from specific species such as rat, mouse, or human can be selected to obtain only those polyclonal antibodies that are specifically immunoreactive with marker “X” and not with other proteins, except for polymorphic variants and alleles of marker “X”. This selection may be achieved by subtracting out antibodies that cross-react with marker “X” molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

As used herein, “humanized antibody of reduced immunogenicity” refers to a humanized antibody exhibiting reduced immunogenicity relative to the parent antibody. Preferably the humanized antibody will exhibit the same or substantially the same antigen-binding affinity and avidity as the parent antibody. Preferably, the affinity of the antibody will at least about 10% of that of the parent antibody. More preferably, the affinity will be at least about 50%, greater than the affinity of the parent antibody. More preferably the affinity will be at least about 100%, 200%, or 500% that of the parent antibody. Methods for assaying antigen-binding affinity are well known in the art and include half-maximal binding assays, competition assays, and Scatchard analysis. Suitable antigen binding assays are described in this application.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the term “safe and effective amount” or “therapeutic amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. For example, an amount effective to delay the growth of or to cause a cancer, either a sarcoma or lymphoma, or to shrink the cancer or prevent metastasis. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

As used herein, a “pharmaceutical salt” include, but are not limited to, mineral or organic acid salts of basic residues such as amines; alkali or organic salts of acidic residues such as carboxylic acids. Preferably the salts are made using an organic or inorganic acid. These preferred acid salts are chlorides, bromides, sulfates, nitrates, phosphates, sulfonates, formates, tartrates, maleates, malates, citrates, benzoates, salicylates, ascorbates, and the like. The most preferred salt is the hydrochloride salt.

As used herein, “cancer” refers to all types of cancer or neoplasm or malignant tumors found in mammals, including, but not limited to: leukemias, lymphomas, melanomas, carcinomas and sarcomas. Examples of cancers are cancer of the brain, breast, pancreas, cervix, colon, head and neck, kidney, lung, non-small cell lung, melanoma, mesothelioma, ovary, sarcoma, stomach, uterus and Medulloblastoma.

Additional cancers which can be treated the chimeric fusion molecule according to the invention include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

“Sample” is used herein in its broadest sense. A sample comprising polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

“Treatment” is an intervention performed with the intention of preventing the development or altering the pathology or symptoms of a disorder. Accordingly, “treatment” refers to both therapeutic treatment and prophylactic or preventative measures. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy. As used herein, “ameliorated” or “treatment” refers to a symptom which is approaches a normalized value (for example a value obtained in a healthy patient or individual), e.g., is less than 50% different from a normalized value, preferably is less than about 25% different from a normalized value, more preferably, is less than 10% different from a normalized value, and still more preferably, is not significantly different from a normalized value as determined using routine statistical tests.

The “treatment of cancer or tumor cells”, refers to an amount of chimeric fusion molecule, described throughout the specification and in the Examples which follow, capable of invoking one or more of the following effects: (1) inhibition, to some extent, of tumor growth, including, (i) slowing down (ii) inhibiting angiogenesis and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

As used herein, “an ameliorated symptom” or “treated symptom” refers to a symptom which approaches a normalized value, e.g., is less than 50% different from a normalized value, preferably is less than about 25% different from a normalized value, more preferably, is less than 10% different from a normalized value, and still more preferably, is not significantly different from a normalized value as determined using routine statistical tests.

As used herein, “metronomic” therapy refers to the administration of continuous low-doses of a therapeutic agent and/or chimeric fusion molecule described herein.)

“Cells of the immune system” or “immune cells” as used herein, is meant to include any cells of the immune system that may be assayed, including, but not limited to, B lymphocytes, also called B cells, T lymphocytes, also called T cells, natural killer (NK) cells, natural killer T (NK) cells, lymphokine-activated killer (LAK) cells, monocytes, macrophages, neutrophils, granulocytes, mast cells, platelets, Langerhans cells, stem cells, dendritic cells, peripheral blood mononuclear cells, tumor-infiltrating (TIL) cells, gene modified immune cells including hybridomas, drug modified immune cells, and derivatives, precursors or progenitors of the above cell types.

“Immune effector cells” refers to cells capable of binding an antigen and which mediate an immune response selective for the antigen. These cells include, but are not limited to, T cells (T lymphocytes), B cells (B lymphocytes), monocytes, macrophages, natural killer (NKT) cells and cytotoxic T lymphocytes (CTLs), for example CTL lines, CTL clones, and CTLs from tumor, inflammatory, or other infiltrates.

“Immune related molecules” refers to any molecule identified in any immune cell, whether in a resting (“non-stimulated”) or activated state, and includes any receptor, ligand, cell surface molecules, nucleic acid molecules, polypeptides, variants and fragments thereof.

A “chemokine” is a small cytokine involved in the migration and activation of cells, including phagocytes and lymphocytes, and plays a role in inflammatory responses.

A “cytokine” is a protein made by a cell that affect the behavior of other cells through a “cytokine receptor” on the surface of the cells the cytokine effects. Cytokines manufactured by lymphocytes are sometimes termed “lymphokines.” Cytokines are also characterized as Type I (e.g. IL-2 and IFN-γ) and Type II (e.g. IL-4 and IL-10).

By the term “modulate,” it is meant that any of the mentioned activities, are, e.g., increased, enhanced, increased, augmented, agonized (acts as an agonist), promoted, decreased, reduced, suppressed blocked, or antagonized (acts as an antagonist). Modulation can increase activity more than 1-fold, 2-fold, 3-fold, 5-fold, 10-fold, 100-fold, etc., over baseline values. Modulation can also decrease its activity below baseline values.

An “epitope”, as used herein, is a portion of a polypeptide that is recognized (i.e., specifically bound) by a B-cell and/or T-cell surface antigen receptor. Epitopes may generally be identified using well known techniques, such as those summarized in Paul, Fundamental Immunology, 3rd ed., 243-247 (Raven Press, 1993) and references cited therein. Such techniques include screening polypeptides derived from the native polypeptide for the ability to react with antigen-specific antisera and/or T-cell lines or clones. An epitope of a polypeptide is a portion that reacts with such antisera and/or T-cells at a level that is similar to the reactivity of the full length polypeptide (e.g., in an ELISA and/or T-cell reactivity assay). Such screens may generally be performed using methods well known to those of ordinary skill in the art, such as those described in Harlow and Lane, Antibodies: A Laboratory Manual, Cold Spring Harbor Laboratory, 1988. B-cell and T-cell epitopes may also be predicted via computer analysis.

“Immunoassay” is an assay that uses an antibody to specifically bind an antigen (e.g., a marker). The immunoassay is characterized by the use of specific binding properties of a particular antibody to isolate, target, and/or quantify the antigen.

“Activity”, “activation” or “augmentation” is the ability of immune cells to respond and exhibit, on a measurable level, an immune function. Measuring the degree of activation refers to a quantitative assessment of the capacity of immune cells to express enhanced activity when further stimulated as a result of prior activation. The enhanced capacity may result from biochemical changes occurring during the activation process that allow the immune cells to be stimulated to activity in response to low doses of stimulants.

“Immune cell activity” as used herein refers to the activation of any immune cell. Activity that may be measured include, but is not limited to, (1) cell proliferation by measuring the DNA replication; (2) enhanced cytokine production, including specific measurements for cytokines, such as IFN-γ, GM-CSF, or TNF-α; (3) cell mediated target killing or lysis; (4) cell differentiation; (5) immunoglobulin production; (6) phenotypic changes; (7) production of chemotactic factors or chemotaxis, meaning the ability to respond to a chemotactin with chemotaxis; (8) immunosuppression, by inhibition of the activity of some other immune cell type; and, (9) apoptosis, which refers to fragmentation of activated immune cells under certain circumstances, as an indication of abnormal activation.

The term “DNA construct” and “vector” are used herein to mean a purified or isolated polynucleotide that has been artificially designed and which comprises at least two nucleotide sequences that are not found as contiguous nucleotide sequences in their natural environment.

As used herein, the term “administering a molecule to a cell” (e.g., an expression vector, nucleic acid, a angiogenic factor, a delivery vehicle, agent, and the like) refers to transducing, transfecting, microinjecting, electroporating, or shooting, the cell with the molecule. In some aspects, molecules are introduced into a target cell by contacting the target cell with a delivery cell (e.g., by cell fusion or by lysing the delivery cell when it is in proximity to the target cell).

A cell has been “transformed”, “transduced”, or “transfected” by exogenous or heterologous nucleic acids when such nucleic acids have been introduced inside the cell. Transforming DNA may or may not be integrated (covalently linked) with chromosomal DNA making up the genome of the cell. In prokaryotes, yeast, and mammalian cells for example, the transforming DNA may be maintained on an episomal element, such as a plasmid. In a eukaryotic cell, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations (e.g., at least about 10).

As used interchangeably herein, the terms “oligonucleotides”, “polynucleotides”, and “nucleic acids” include RNA, DNA, or RNA/DNA hybrid sequences of more than one nucleotide in either single chain or duplex form. The term “nucleotide” as used herein as an adjective to describe molecules comprising RNA, DNA, or RNA/DNA hybrid sequences of any length in single-stranded or duplex form. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. Although the term “nucleotide” is also used herein to encompass “modified nucleotides” which comprise at least one modifications (a) an alternative linking group, (b) an analogous form of purine, (c) an analogous form of pyrimidine, or (d) an analogous sugar, all as described herein.

As used herein, “molecule” is used generically to encompass any vector, antibody, protein, drug and the like which are used in therapy and can be detected in a patient by the methods of the invention. For example, multiple different types of nucleic acid delivery vectors encoding different types of genes which may act together to promote a therapeutic effect, or to increase the efficacy or selectivity of gene transfer and/or gene expression in a cell. The nucleic acid delivery vector may be provided as naked nucleic acids or in a delivery vehicle associated with one or more molecules for facilitating entry of a nucleic acid into a cell. Suitable delivery vehicles include, but are not limited to: liposomal formulations, polypeptides; polysaccharides; lipopolysaccharides, viral formulations (e.g., including viruses, viral particles, artificial viral envelopes and the like), cell delivery vehicles, and the like.

As used herein, the term “oligonucleotide” refers to a polynucleotide formed from naturally occurring bases and pentofuranosyl groups joined by native phosphodiester bonds. This term effectively refers to naturally occurring species or synthetic species formed from naturally occurring subunits or their close homologs. The term “oligonucleotide” may also refer to moieties which function similarly to naturally occurring oligonucleotides but which have non-naturally occurring portions. Thus, oligonucleotides may have altered sugar moieties or intersugar linkages. Exemplary among these are the phosphorothioate and other sulfur-containing species which are known for use in the art. In accordance with some preferred embodiments, at least some of the phosphodiester bonds of the oligonucleotide have been substituted with a structure which functions to enhance the ability of the compositions to penetrate into the region of cells where the RNA or DNA whose activity to be modulated is located. It is preferred that such substitutions comprise phosphorothioate bonds, methyl phosphonate bonds, or short chain alkyl or cycloalkyl structures. In accordance with other preferred embodiments, the phosphodiester bonds are substituted with other structures which are, at once, substantially non-ionic and non-chiral, or with structures which are chiral and enantiomerically specific. Persons of ordinary skill in the art will be able to select other linkages for use in practice of the invention.

Oligonucleotides may also include species which include at least some modified base forms. Thus, purines and pyrimidines other than those normally found in nature may be so employed. Similarly, modifications on the pentofuranosyl portion of the nucleotide subunits may also be effected, as long as the essential tenets of this invention are adhered to. Examples of such modifications are 2′-O-alkyl- and 2′-halogen-substituted nucleotides. Some specific examples of modifications at the 2′ position of sugar moieties which are useful in the present invention are OH, SH, SCH₃, F, OCH₃, OCN, O(CH₂)ₙNH₂ or O(CH₂)ₙCH₃ where n is from 1 to about 10, and other substituents having similar properties.

In a preferred embodiment, a composition is provided comprising a therapeutically effective anti-tumor molecule fused to a constant region domain of an antibody. By way of illustration, the composition comprises an anti-tumor antibody specific, for example, the HER2/neu tumor antigen, in which endostatin is fused to the C_{H}3 domain of human IgG3 antibody.

In general, the invention provides antigen-binding fusion proteins with a modulatory or cytolytic moiety which have significant serum half-life (t_{1/2}) beyond that of (either antibody) modulatory/cytolytic moiety alone. Modulatory and cytolytic antigen-binding fusion proteins have more than an antigen-binding site activity or function. A modulatory or cytolytic moiety on the fusion antigen-binding protein will impart upon the protein certain or all of the modulatory or cytolytic attributes of the fusion partner or partners.

Accordingly, the invention is directed to single-chain and multivalent modulatory and cytolytic antigen-binding fusion proteins, compositions of single-chain and multivalent modulatory and cytolytic antigen-binding fusion proteins, methods of making and purifying single-chain and multivalent modulatory and cytolytic antigen-binding fusion proteins, and uses for single-chain and multivalent modulatory and cytolytic antigen-binding fusion proteins. The invention provides a modulatory or cytolytic antigen-binding fusion protein having at least one single-chain antigen-binding protein molecule. Each single-chain antigen-binding molecule has a first polypeptide and a second polypeptide joined by a linker. Each of the polypeptides has the binding portion of the variable region of an antibody heavy or light chain.

As an illustrative example which is not meant to limit or construe the invention in any way, the following is provided.

To provide a modulatory molecule for fusion to an antibody molecule, such as for example, endostatin, the endostatin gene is first isolated and amplified. In this illustrative example, the endostatin gene originated from pFLAG-CMV-1-endostatin by PCR using primers 5′-CCCCTCGCGATATCATACTCATCAGGACTTTCAGCC-3′ (SEQ ID NO 1) and 5′-CCCCGAATTCGTTAACCTTTGGAGAAAGAGGTCATGAAGC-3′ (SEQ ID NO 2). PCR products were subcloned into, for example, p-GEM-T Easy Vector (Promega, Madison, Wis.), then sequenced for verification. The EcORV-EcOR1 fragment of the subcloned endostatin gene was ligated to the carboxyl end of the heavy chain constant domain (C_{H}3) of human IgG3 in the vector, for example, pAT135. To complete the construct, the IgG3-endostatin heavy chain constant region (AgeI-BamHI) was then joined to an anti-HER2/neu variable region of a recombinant humanized monoclonal antibody, for example, 4D5-8 (rhuMAb HER2, Herceptin; Genentech, San Francisco, Calif.) in the expression vector (pSV2-his) containing HisD gene for eukaryotic selection. The finished anti-HER2/neu heavy chain IgG3-endostatin construction vector was transfected by electroporation into, for example, Sp2/0 cells stably expressing the anti-HER2/neu K light chain in order to assemble entire anti-HER2/neu IgG3-endostatin fusion proteins. Transfected cells were selected, for example, with 5 mM histidinol and transfectomas producing the fusion proteins were identified by a enzyme-linked immunosorbent assay (ELISA) using anti-human IgG antibody coated plates and an anti-human kappa detection antibody (Sigma, Saint Louis, Mo.). The anti-HER2/neu IgG3-endostatin fusion proteins were biosynthetically labeled with [³⁵S]methionine (Amersham Biosciences, Piscataway, N.J.) and analyzed by SDS-PAGE on 5% sodium phosphate buffered polyacrylamide gels without reduction or on 12.5% Tris-glycine buffered polyacrylamide gels following treatment with 0.15 M β-mercaptoethanol at 37° C. for 30 min. The fusion protein was purified from culture supernatants using protein A immobilized on Sepharose 4B fast flow (Sigma, Saint Louis, Mo.).

To obtain active endostatin, a mouse endostatin expression vector, for example, pFLAG-CMV-1-endostatin was co-transfected with, for example, pcDNA3.1 (CLONTECH, Palo Alto, Calif.) into human embryonic kidney (HEK) 293 cells, and G418 (0.6 μg/ml)-resistant cells. Secreted endostatin was harvested from serum-free conditioned medium and purified in a heparin-Sepharose CL-6B column. Purity was assessed by Coomassie blue staining of the SDS-PAGE gels. For Western blot analysis, the endostatin fusion proteins were treated with β-mercaptoethanol, fractionated by SDS-PAGE and transferred onto a membrane. Rabbit anti-endostatin (BodyTech, Kangwon-Do, Korea) was used as the primary antibody and mouse anti-rabbit IgG conjugated with HRP (Sigma, St. Louis, Mo.) was used as the secondary antibody. Goat anti-human IgG conjugated with HRP (Sigma, Saint Louis, Mo.) was used to detect human antibody.

Methods involving conventional biological techniques are described herein. Such techniques are generally known in the art and are described in detail in methodology treatises such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992.

In another preferred embodiment, the invention provides administering the antibody-fusion molecule with a cocktail of one or more compounds such as for example, endostatin, angiogenin, angiostatin, chemokines, angioarrestin, angiostatin (plasminogen fragment), basement-membrane collagen-derived anti-angiogenic factors (tumstatin, canstatin, or arrestin), anti-angiogenic antithrombin III, cartilage-derived inhibitor (CDI), CD59 complement fragment, fibronectin fragment, gro-beta, heparinases, heparin hexasaccharide fragment, human chorionic gonadotropin (hCG), interferon alpha/beta/gamma, interferon inducible protein (IP-10), interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-b), vasculostatin, vasostatin (calreticulin fragment) and the like.

Cytolytic molecules that can be used to fuse to an antibody or fragment thereof, include, but are not limited to TNF-α, TNF-β, suitable effector genes such as those that encode a peptide toxin—such as ricin, abrin, diphtheria, gelonin, Pseudomonasexotoxin A, Crotalus durissus terrificus toxin, Crotalus adamenteus toxin, Naja naja toxin, and Naja mocambique toxin. (Hughes et al., Hum. Exp. Toxicol. 15:443, 1996; Rosenblum et al., Cancer Immunol. Immunother. 42:115, 1996; Rodriguez et al., Prostate 34:259, 1998; Mauceri et al., Cancer Res. 56:4311; 1996).

Also suitable are genes that induce or mediate apoptosis—such as the ICE-family of cysteine proteases, the Bcl-2 family of proteins, Bax, bclXs and caspases (Favrot et al., Gene Ther. 5:728, 1998; McGill et al., Front. Biosci. 2:D353, 1997; McDonnell et al., Semin. Cancer Biol. 6:53, 1995). Another potential anti-tumor agent is apoptin, a protein that induces apoptosis even where small drug chemotherapeutics fail (Pietersen et al., Adv. Exp. Med. Biol. 465:153, 2000). Koga et al. (Hu. Gene Ther. 11: 1397, 2000) propose a telomerase-specific gene therapy using the hTERT gene promoter linked to the apoptosis gene Caspase-8 (FLICE).

Also of interest are enzymes present in the lytic package that cytotoxic T lymphocytes or LAK cells deliver to their targets. Perforin, a pore-forming protein, and Fas ligand are major cytolytic molecules in these cells (Brandau et al., Clin. Cancer Res. 6:3729, 2000; Cruz et al., Br. J. Cancer 81:881, 1999). CTLs also express a family of at least 11 serine proteases termed granzymes, which have four primary substrate specificities (Kam et al., Biochim. Biophys. Acta 1477:307, 2000). Low concentrations of streptolysin 0 and pneumolysin facilitate granzyme B-dependent apoptosis (Browne et al., Mol. Cell Biol. 19:8604, 1999).

Other suitable effectors encode polypeptides having activity that is not itself toxic to a cell, but renders the cell sensitive to an otherwise nontoxic compound—either by metabolically altering the cell, or by changing a non-toxic prodrug into a lethal drug. Exemplary is thymidine kinase (tk), such as may be derived from a herpes simplex virus, and catalytically equivalent variants. The HSV tk converts the anti-herpetic agent ganciclovir (GCV) to a toxic product that interferes with DNA replication in proliferating cells.

If desired, although not required, factors may also be included, such as, but not limited to, interleukins, e.g. IL-2, IL-3, IL-6, and IL-11, as well as the other interleukins, the colony stimulating factors, such as GM-CSF, interferons, e.g. γ-interferon, erythropoietin.

In another preferred embodiment, the invention provides for antibody fusion molecules comprising a modulatory or cytotoxic molecule fused to the F_{c} region, C_{H}1, C_{H}2 and/or C_{H}3, Fab, Fab′, F(ab′)₂, single chain Fv (S_{c}Fv) and Fv fragments, as well as any portion of an antibody having specificity toward a desired target epitope or epitopes.

domains of the antibody. Also preferred are antibodies or antibody fragments or to single chain, two-chain, and multi-chain proteins and glycoproteins belonging to the classes of polyclonal, monoclonal, chimeric, bispecific and hetero immunoglobulins (monoclonal antibodies being preferred); it also includes synthetic and genetically engineered variants of these immunoglobulins.

In another preferred embodiment, carrier domains within the invention can be used to introduce an effector function to the chimeric molecule. For introducing an effector function to the chimeric molecule, the carrier domain can be a protein that has been shown to possess cytotoxic or immune response-stimulating properties. For instance, carrier domains for introducing a cytotoxic function to the chimeric molecule include a bacterial toxin, ricin, abrin, saporin, pokeweed viral protein, and constant region domains from an immunoglobulin molecule (e.g., for antibody dependent cell-mediated cytotoxicity). Chimeric molecules that contain a cytotoxic carrier domain can be used to selectively kill cells.

For introducing immune response-stimulating properties to a chimeric molecule, carrier domains within the invention include any known to activate an immune system component. For example, antibodies and antibody fragments (e.g., CH₂—CH₃) can be used as a carrier domain to engage Fc receptors or to activate complement components. A number of other immune system-activating molecules are known that might also be used as a carrier domain, e.g., microbial superantigens, adjuvant components, lipopolysaccharide (LPS), and lectins with mitogenic activity. Other carrier domains that can be used to introduce an effector function to the chimeric molecule can be identified using known methods. For instance, a molecule can be screened for suitability as a carrier domain by fusing the molecule to an anti-angiogenic agent and testing the chimeric molecule in in vitro or in vivo cell cytotoxicity and humoral response assays.

Other Tumor Antigens

In another preferred embodiment, the chimeric fusion molecules comprise a modulatory or cytolytic molecule, as described above, to an antibody or fragment thereof, specific for other tumor antigens. Many tumor antigens are well known in the art. See for example, Van den Eynde B J, van der Bruggen P. Curr Opin Immunol 1997; 9: 684-93; Houghton A N, Gold J S, Blachere N E. Curr Opin Immunol 2001; 13: 134-140; van der Bruggen P, Zhang Y, Chaux P, Stroobant V, Panichelli C, Schultz E S, Chapiro J, Van den Eynde B J, Brasseur F, Boon T. Immunol Rev 2002; 188: 51-64, which are herein incorporated by reference in their entirety. Alternatively, many antibodies directed towards tumor antigens are commercially available.

Non-limiting examples of tumor antigens, include, tumor antigens resulting from mutations, such as: alpha-actinin-4 (lung carcinoma); BCR-ABL fusion protein (b3a2) (chronic myeloid leukemia); CASP-8 (head and neck squamous cell carcinoma); beta-catenin (melanoma); Cdc27 (melanoma); CDK4 (melanoma); dek-can fusion protein (myeloid leukemia); Elongation factor 2 (lung squamous carcinoa); ETV6-AML1 fusion protein (acute lymphoblastic leukemia); LDLR-fucosyltransferaseAS fusion protein (melanoma); overexpression of HLA-A2^{d} (renal cell carcinoma); hsp70-2 (renal cell carcinoma); KIAAO205 (bladder tumor); MART2 (melanoma); MUM-1 f (melanoma); MUM-2 (melanoma); MUM-3 (melanoma); neo-PAP (melanoma); Myosin class I (melanoma); OS-9g (melanoma); pml-RARalpha fusion protein (promyelocytic leukemia); PTPRK (melanoma); K-ras (pancreatic adenocarcinoma); N-ras (melanoma). Examples of differentiation tumor antigens include, but not limited to: CEA (gut carcinoma); gp100/Pmell7 (melanoma); Kallikrein 4 (prostate); mammaglobin-A (breast cancer); Melan-A/MART-I (melanoma); PSA (prostate carcinoma); TRP-1/gp75 (melanoma); TRP-2 (melanoma); tyrosinase (melanoma). Over or under-expressed tumor antigens include but are not limited to: CPSF (ubiquitous); EphA3; G250/MN/CAIX (stomach, liver, pancreas); HER-2/neu; Intestinal carboxyl esterase (liver, intestine, kidney); alpha-foetoprotein (liver); M-CSF (liver, kidney); MUCI (glandular epithelia); p53 (ubiquitous); PRAME (testis, ovary, endometrium, adrenals); PSMA (prostate, CNS, liver); RAGE-I (retina); RU2AS (testis, kidney, bladder); survivin (ubiquitous); Telomerase (testis, thymus, bone marrow, lymph nodes); WT1 (testis, ovary, bone marrow, spleen); CA125 (ovarian).

Anti-Angiogenic Chimeric Molecules

In another preferred embodiment, the invention provides chimeric molecules that include both an anti-angiogenic agent domain and carrier domain. The anti-angiogenic agent domain reduces tumor growth (e.g., by inhibiting angiogenesis), while the carrier domain confers a functional attribute to the chimeric molecule. For instance, where the carrier domain is an Ig domain, it can function to target the chimeric molecule to a particular site (e.g., the antigen-binding portion of the antibody binds to an antigen expressed by a target cell and/or the Fc portion of the Ig domain can target the chimeric molecule to an Fc receptor-bearing cell); to increase stability of the chimeric molecule (e.g., for in vitro storage or in vivo delivery); to impart an effector function to the chimeric molecule (e.g., immune response-stimulating, cytotoxicity, etc.); or to facilitate purification of the chimeric molecule.

The experiments described below utilize endostatin as the anti-angiogenic agent domain. Nonetheless, any other substance that exerts an anti-angiogenic effect might be used as the anti-angiogenic agent, e.g., anti-angiogenic chemokines, angioarrestin, angiostatin (plasminogen fragment), basement-membrane collagen-derived anti-angiogenic factors (tumstatin, canstatin, or arrestin), anti-angiogenic antithrombin III, cartilage-derived inhibitor (CDI), CD59 complement fragment, fibronectin fragment, gro-beta, heparinases, heparin hexasaccharide fragment, human chorionic gonadotropin (hCG), interferon alpha/beta/gamma, interferon inducible protein (IP-10), interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-b), vasculostatin, vasostatin (calreticulin fragment), and other naturally occurring or man-made inhibitors of neovascularization.

The anti-angiogenic agent can be an intact molecule, a functionally fragment of the agent, or a naturally occurring or man-made mutant of the agent. For example, endostatin domains useful in the invention include any molecule derived from a native endostatin that shares a functional activity of endostatin, e.g., the ability to inhibit VEGF production or new vessel formation The endostatin domain can be a native endostatin or a fragment of a native endostatin that retains a functional activity of a native endostatin. The endostatin domain can also be a non-naturally occurring form a endostatin (e.g., a mutant form created by amino acid substitution) that retains a functional activity of a native endostatin.

The carrier domain can be any substance that imparts a function to the chimeric molecule. For example, a carrier domain can be a molecule that increases the stability of the chimeric molecule (e.g., for in vitro storage or in vivo delivery); introduces an effector function to the chimeric molecule (e.g., immune response-stimulating, cytotoxicity, etc.); or facilitates purification of the chimeric molecule. For increasing the stability of the chimeric molecule, the carrier domain can be a protein that has been shown to stabilize molecules in an in vitro storage or in vivo delivery setting. For example, carrier domains for increasing the stability of the chimeric molecule include one or more domains from an Ig molecule (e.g., a CH₂-CH₃ fragment). Other carrier domains that can be used to stabilize the chimeric molecule can be identified empirically. For instance, a molecule can be screened for suitability as a carrier domain by conjugating the molecule to anti-angiogenic agent and testing the conjugated product in in vitro or in vivo stability assays.

In another preferred embodiment, carrier domains within the invention facilitate purification of the chimeric molecule. Any molecule known to facilitate purification of a chimeric molecule can be used. Representative examples of such carrier domains include antibody fragments and affinity tags (e.g., GST, HIS, FLAG, and HA). Chimeric molecules containing an affinity tag can be purified using immunoaffinity techniques (e.g., agarose affinity gels, glutathione-agarose beads, antibodies, and nickel column chromatography). Chimeric molecules that contain an Ig domain as a carrier domain can be purified using immunoaffinity chromatography techniques known in the art (e.g., protein A or protein G chromatography).

Other carrier domains within the invention that can be used to purify the chimeric molecule can be readily identified by testing the molecules in a functional assay. For instance, a molecule can be screened for suitability as a carrier domain by fusing the molecule to an anti-angiogenic agent and testing the fusion for purity and yield in an in vitro assay. The purity of recombinant proteins can be estimated by conventional techniques, for example, SDS-PAGE followed by the staining of gels with Coomassie-Blue.

A number of other carrier domains can be used to impart an effector function to the chimeric molecule. These include other cytotoxins, drugs, detectable labels, targeting ligands, and delivery vehicles. Examples of these are described in U.S. Pat. No. 6,518,061 and U.S. published patent application number 20020159972.

A preferred carrier domain for use in the chimeric molecule is an Ig or portion of an Ig. The Ig domain might take the form of a single chain antibody (e.g., a scFV), an Fab fragment, an F(ab′)₂ fragment, an Ig heavy chain, or an Ig in which one or more of the constant regions has been removed. The Ig domain can be derived from any Ig class including IgG, IgM, IgE, IgA, IgD and any subclass thereof. In some applications, it is preferred that the Ig domain includes a large hinge region, e.g., one from IgG3.

In another preferred embodiment, the Ig domain is a minibody. A small protein scaffold called a “minibody” was designed using a part of the Ig VH domain as the template (Pessi et al., 1993). Minibodies with high affinity (dissociation constant (K_{d}) about 10⁻⁷ M) to interleukin-6 were identified by randomizing loops corresponding to CDR1 and CDR2 of VH and then selecting mutants using the phage display method (Martin et al., 1994). These experiments demonstrated that the essence of the Ab function could be transferred to a smaller system. Thus, the chimeric fusion molecule may comprise a minibody Ig domain.

Chimeric molecules can be prepared using conventional techniques in molecular biology or protein chemistry. Where the chimeric molecule is a fusion protein, molecular biology methods can be used to join two or more genes in frame into a single nucleic acid. The nucleic acid can then be expressed in an appropriate host cell under conditions in which the chimeric molecule is produced. A carrier domain might also be conjugated (e.g., covalently bonded) to an anti-angiogenic agent domain by other methods known in the art for conjugating two such molecules together. For example, the anti-angiogenic agent domain can be chemically derivatized with a carrier domain either directly or using a linker (spacer). Several methods and reagents (e.g., cross-linkers) for mediating this conjugation are known. See, e.g., catalog of Pierce Chemical Company; and Means and Feeney, Chemical Modification of Proteins, Holden-Day Inc., San Francisco, Calif. 1971; “Monoclonal Antibody-Toxin Conjugates: Aiming the Magic Bullet,” Thorpe et al., Monoclonal Antibodies in Clinical Medicine, Academic Press, pp. 168-190 (1982); Waldmann (1991) Science, 252: 1657; and U.S. Pat. Nos. 4,545,985 and 4,894,443.

An anti-angiogenic agent domain may be fused or conjugated to a carrier domain in various orientations. For example, the carrier domain may be joined to either the amino or carboxy termini of an anti-angiogenic agent domain. The anti-angiogenic agent domain may also be joined to an internal region of the carrier domain, or conversely, the carrier domain may be joined to an internal location of the anti-angiogenic agent domain.

In some circumstances, it is desirable to free the carrier domain from the anti-angiogenic agent domain when the chimeric molecule has reached its target site. Therefore, chimeric conjugates featuring linkages that are cleavable in the vicinity of the target site may be used when one of the domains is to be released at the target site. Cleaving of the linkage to release the carrier domain from the anti-angiogenic agent domain may be prompted by enzymatic activity or conditions to which the conjugate is subjected either inside the target cell or in the vicinity of the target site. When the target site is a tumor, a linker which is cleavable under conditions present at the tumor site (e.g. when exposed to tumor-associated enzymes or acidic pH) may be used. A number of different cleavable linkers are known to those of skill in the art. See, e.g., U.S. Pat. Nos. 4,618,492; 4,542,225; and 4,625,014. The mechanisms for release of an agent from these linker groups include, for example, irradiation of a photolabile bond and acid-catalyzed hydrolysis. U.S. Pat. No. 4,671,958, for example, includes a description of immunoconjugates comprising linkers which are cleaved at the target site in vivo by the proteolytic enzymes of the patient's complement system. In view of the large number of methods that have been reported for attaching a variety of radiodiagnostic compounds, radiotherapeutic compounds, drugs, toxins, and other agents to proteins one skilled in the art will be able to determine a suitable method for attaching a given carrier domain to an anti-angiogenic agent domain.

Bispecific Chimeric Molecules

In another preferred embodiment, chimeric molecules comprising a modulatory or cytolytic domain is fused to a bispecific antibody domain or fragments thereof. In one aspect of the invention, the bispecific antibody comprises two monoclonal antibodies. However, the bispecific antibody can comprise two polyclonal antibodies or an engineered bispecific antibody.

Preferably, each of the specificities of the bispecific antibody are directed to one or more tumor antigens and/or specific cell or tissue. Antibodies can be raised against any tumor antigen from a patient. Thus the targeting of the chimeric molecule can be individually tailored as the tumor displays different antigens.

Bispecific antibodies may be constructed by hybrid-hybridoma techniques, by covalently linking specific antibodies or by other approaches, like the diabody approach (Kipriyanow, Int. J. Cancer 77 (1998), 763-773). In one aspect of the invention, the bispecific antibody is a single chain antibody construct.

For tracking purposes, the bispecific antibody can be directly labeled or a second antibody specific for a region of the bispecific antibody is labeled. Detection of the localization of the chimeric molecule is preferably through cell sorting techniques such as flow cytometry. For example, wherein samples are taken at different time intervals after administration of the chimeric molecule for imaging and diagnostic purposes.

In accordance with the invention, the bispecific antibody, targets chimeric molecules to a specific location in vivo. For example, the location can be to myocardial tissues, breast, liver, spleen, ovaries, testis, hepatocyte, kidneys and the like. The bispecific antibody determines the specific antigen to which the chimeric molecule is targeted.

As described above, the specificity of the antibody domain can be directed to a specific tissue antigen wherein the tumor has been detected coupled with specificity for that particular tumor antigen. Alternatively, the bispecific antibody domain is directed to two tumor antigens that are expressed by the tumor. The bispecific domain can be fused to any modulatory or cytolytic domain discussed above.

In another embodiment of the invention, the bispecific antibody (BiAb) construct is a bispecific antibody that binds to one or more tumor antigens as a first or second antigen and a cell or tissue specific antigen a second antigen. The antibody may be covalently bound to the a modulatory or cytolytic molecule and the chimeric molecule may be constructed by chemical coupling, producing a fusion protein or a mosaic protein from the antibody and from a modified or unmodified prokaryotic or eukaryotic modulatory or cytotoxic molecule. Furthermore, the antibody may be joined to modulatory or cytotoxic molecule via multimerization domains.

In another embodiment of the invention, the chimeric polypeptide of the invention, e.g., a endostatin construct, is a fusion construct of a modified or an unmodified endostatin with a modified or an unmodified modulatory or cytotoxic molecule. The construct may be bound in vitro and/or in vivo, e.g., by a multimerization domain, to bispecific antibody domain. The chimeric molecule constructs may, inter alia, result from chemical coupling, may be recombinantly produced (as shown in the appended examples), or may be produced as a fusion protein as described above. In one aspect, the moiety specifically binds to at least one tumor antigen.

The compositions of the invention can comprise any cytotoxic agent as described infra. For example, in one aspect, the toxin may be a polypeptide toxin, e.g., a Pseudomonasexotoxin, like PE38, PE40 or PE37, or a truncated version thereof, or a ribosome inactivating protein gelonin (e.g., Boyle (1996) J. Immunol. 18:221-230), and the like. The compositions of the invention can be conjugated to any cytotoxic pharmaceuticals, e.g., radiolabeled with a cytotoxic agents, such as, e.g., 0.1311 (e.g., Shen (1997) Cancer 80(12 Suppl):2553-2557), copper-67 (e.g., Deshpande (1988) J. Nucl. Med. 29:217-225).

In one embodiment, the chimeric molecule construct is a fusion (poly)peptide or a mosaic (poly)peptide. The fusion (poly)peptide may comprise merely the domains of the constructs as described herein, as well as (a) functional fragment(s) thereof. However, it is also envisaged that the fusion (poly)peptide comprises further domains and/or functional stretches. Therefore, the fusion (poly)peptide can comprise at least one further domain, this domain being linked by covalent or non-covalent bonds. The linkage as well as the construction of such constructs, can be based on genetic fusion according to the methods described herein or known in the art (e.g., Sambrook et al., loc. cit., Ausubel, “Current Protocols in Molecular Biology”, Green Publishing Associates and Wiley Interscience, N.Y. (1989)) or can be performed by, e.g., chemical cross-linking as described in, e.g., WO 94/04686. The additional domain present in the construct may be linked by a flexible linker, such as a (poly)peptide linker, wherein the (poly)peptide linker can comprises plural, hydrophilic, peptide-bonded amino acids of a length sufficient to span the distance between the C-terminal end of the further domain and the N-terminal end of the peptide, (poly)peptide or antibody or vice versa. The linker may, inter alia, be a Glycine, a Serine and/or a Glycine/Serine linker. Additional linkers comprise oligomerization domains. Oligomerization domains can facilitate the combination of two or several antigens or fragments thereof in one functional molecule. Non-limiting examples of oligomerization domains comprise leucine zippers (like jun-fos, GCN4, E/EBP; Kostelny, J. Immunol. 148 (1992), 1547-1553; Zeng, Proc. Natl. Acad. Sci. USA 94 (1997), 3673-3678, Williams, Genes Dev. 5 (1991), 1553-1563; Suter, “Phage Display of Peptides and Proteins”, Chapter 11, (1996), Academic Press), antibody-derived oligomerization domains, like constant domains C_{H}1 and C_{L} (Mueller, FEBS Letters 422 (1998), 259-264) and/or tetramerization domains like GCN₄-LI (Zerangue, Proc. Natl. Acad. Sci. USA 97 (2000), 3591-3595).

Furthermore, the chimeric fusion construct to be used in the present invention, as described herein, may comprise at least one further domain, inter alia, domains which provide for purification means, like, e.g. histidine stretches. The further domain(s) may be linked by covalent or non-covalent bonds.

The linkage can be based on genetic fusion according to the methods known in the art and described herein or can be performed by, e.g., chemical cross-linking as described in, e.g., WO 94/04686. The additional domain present in the construct may be linked by a flexible linker, such as a polypeptide linker to one of the binding site domains; the polypeptide linker can comprise plural, hydrophilic or peptide-bonded amino acids of a length sufficient to span the distance between the C-terminal end of one of the domains and the N-terminal end of the other of the domains when the polypeptide assumes a conformation suitable for binding when disposed in aqueous solution.

Immune Activating Chimeric Fusion Molecules

It is also envisaged that the constructs disclosed for uses, compositions and methods of the present invention comprises (a) further domain(s) which may function as immunomodulators. The immunomodulators comprise, but are not limited to cytokines, lymphokines, T cell co-stimulatory ligands, etc. Preferably, the chimeric fusion molecule targets and delivers a modulatory or cytolytic molecule to the tumor cell and also recruits immune cells and/or activated immune cells to the tumor.

Adequate activation resulting in priming of naïve T-cells is critical to primary immunoresponses and depends on two signals derived from professional APCs (antigen-presenting cells) like dendritic cells. The first signal is antigen-specific and normally mediated by stimulation of the clonotypic T-cell antigen receptor (TCR) that is induced by processed antigen presented in the context of MHC class-I or MHC class-II molecules. However, this primary stimulus is insufficient to induce priming responses of naïve T-cells, and the second signal is required which is provided by an interaction of specific T-cell surface molecules binding to co-stimulatory ligand molecules on antigen presenting cells (APCs), further supporting the proliferation of primed T-cells. The term “T-cell co-stimulatory ligand” therefore denotes in the light of the present invention molecules, which are able to support priming of naïve T-cells in combination with the primary stimulus and include, but are not limited to, members of the B7 family of proteins, including B7-1 (CD80) and B7-2 (CD86), 4-1BB ligand, CD40 ligand, OX40 ligand.

The chimeric fusion molecule construct described herein may comprise further receptor or ligand function(s), and may comprise immuno-modulating effector molecule or a fragment thereof. An immuno-modulating effector molecule positively and/or negatively influences the humoral and/or cellular immune system, particularly its cellular and/or non-cellular components, its functions, and/or its interactions with other physiological systems. The immuno-modulating effector molecule may be selected from the group comprising cytokines, chemokines, macrophage migration inhibitory factor (MIF; as described, inter alia, in Bernhagen (1998), Mol Med 76(3-4); 151-61 or Metz (1997), Adv Immunol 66, 197-223), T-cell receptors and soluble MHC molecules. Such immuno-modulating effector molecules are well known in the art and are described, inter alia, in Paul, “Fundamental immunology”, Raven Press, New York (1989). In particular, known cytokines and chemokines are described in Meager, “The Molecular Biology of Cytokines” (1998), John Wiley & Sons, Ltd., Chichester, West Sussex, England; (Bacon (1998). Cytokine Growth Factor Rev 9(2):167-73; Oppenheim (1997). Clin Cancer Res 12, 2682-6; Taub, (1994) Ther. Immunol. 1(4), 229-46 or Michiel, (1992). Semin Cancer Biol 3(1), 3-15).

Immune cell activity that may be measured include, but is not limited to, (1) cell proliferation by measuring the DNA replication; (2) enhanced cytokine production, including specific measurements for cytokines, such as IFN-γ, GM-CSF, or TNF-α; (3) cell mediated target killing or lysis; (4) cell differentiation; (5) immunoglobulin production; (6) phenotypic changes; (7) production of chemotactic factors or chemotaxis, meaning the ability to respond to a chemotactin with chemotaxis; (8) immunosuppression, by inhibition of the activity of some other immune cell type; and, (9) apoptosis, which refers to fragmentation of activated immune cells under certain circumstances, as an indication of abnormal activation.

Modified Chimeric Molecules

The constructs of the present invention may comprise domains originating from one species, e.g., from mammals, such as human. However, chimeric and/or humanized constructs are also envisaged and within the scope of the present invention.

Furthermore, the polynucleotide/nucleic acid molecules of the invention may comprise, for example, thioester bonds and/or nucleotide analogues. The modifications may be useful for the stabilization of the nucleic acid molecule, e.g., against endo- and/or exonucleases in the cell. These nucleic acid molecules may be transcribed by an appropriate vector containing a chimeric gene which allows for the transcription of the nucleic acid molecule in the cell. The polynucleotide/nucleic acid molecules of the invention may be a recombinantly produced chimeric nucleic acid molecule comprising any of the aforementioned nucleic acid molecules either alone or in combination. The polynucleotide may be, e.g., DNA, cDNA, RNA or synthetically produced DNA or RNA or a recombinantly produced chimeric nucleic acid molecule comprising any of those polynucleotides either alone or in combination. The polynucleotide can be part of a vector, e.g., an expression vector, including, e.g., recombinant viruses. The vectors may comprise further genes, such as marker genes, that allow for the selection of the vector in a suitable host cell and under suitable conditions.

In one aspect, the polynucleotides of the invention are operatively linked to expression control sequences allowing expression in prokaryotic or eukaryotic cells. Expression of the polynucleotide comprises transcription of the polynucleotide into a translatable mRNA. Regulatory elements ensuring expression in cells, including eukaryotic cells, such as mammalian cells, are well known to those skilled in the art. They usually comprise regulatory sequences ensuring initiation of transcription, and, optionally, poly-A signals ensuring termination of transcription and stabilization of the transcript. Additional regulatory elements may include transcriptional as well as translational enhancers, and/or naturally-associated or heterologous promoter regions. Exemplary regulatory elements permitting expression in prokaryotic host cells comprise, e.g., the PL, lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. The nucleic acids of the invention can also comprise, in addition to elements responsible for the initiation of transcription, other elements, such regulatory elements and transcription termination signals, such as the SV40-poly-A site or the tk-poly-A site (termination sequences are typically downstream of the polynucleotide coding sequence). Furthermore, depending on the expression system used, nucleic acid sequences encoding leader sequences capable of directing the polypeptide to a cellular compartment, or secreting it into the medium, may be added to the coding sequence of the polynucleotide of the invention; such leader sequences are well known in the art. The leader sequence(s) is (are) assembled in appropriate phase with translation, initiation and termination sequences. In one aspect, the leader sequence is capable of directing secretion of translated chimeric protein, or a portion thereof, into the periplasmic space or extracellular medium. Optionally, the heterologous sequence can encode a fusion protein including an N-terminal identification peptide imparting desired characteristics, e.g., stabilization or simplified purification of expressed recombinant product; see supra. In this context, suitable expression vectors are known in the art such as Okayama-Berg cDNA expression vector pcDV1 (Pharmacia), pCDM8, pRc/CMV, pcDNA1, pcDNA3 (In-vitrogene), or pSPORT1 (GIBCO BRL). Expression control sequences can be eukaryotic promoter systems in vectors capable of transforming or transfecting eukaryotic host cells; control sequences for prokaryotic hosts may also be used. Once the vector has been incorporated into the appropriate host, the host can be maintained under conditions suitable for high level expression of the nucleotide sequences; and, as desired, the collection and purification of the polypeptide of the invention may follow; see, e.g., the appended examples.

As described above, the polynucleotide of the invention can be used alone or as part of a vector (e.g., an expression vector or a recombinant virus), or in cells, to express the chimeric fusion molecules of the invention. The polynucleotides or vectors containing the DNA sequence(s) encoding any one of the chimeric fusion molecules of the invention can be introduced into the cells, which in turn produce the polypeptide of interest.

The present invention is directed to vectors, e.g., plasmids, cosmids, viruses and bacteriophages, or any expression system used conventionally in genetic engineering, that comprise a polynucleotide encoding a chimeric fusion molecule of the invention. The vector can be an expression vector and/or a gene transfer or targeting vector. Expression vectors derived from viruses such as retroviruses, vaccinia virus, adeno-associated virus, herpes viruses, or bovine papilloma virus, may be used for delivery of the polynucleotides or vectors of the invention into targeted cell populations. Methods which are well known to those skilled in the art can be used to construct recombinant vectors; see, for example, the techniques described in Sambrook, Molecular Cloning: A Laboratory Manual, Cold Spring Harbor Laboratory (1989) N.Y. and Ausubel, Current Protocols in Molecular Biology, Green Publishing Associates and Wiley Interscience, N.Y. (1989). Alternatively, the polynucleotides and vectors of the invention can be reconstituted into liposomes for delivery to target cells. The vectors containing the polynucleotides of the invention can be transferred into the host cell by well-known methods, which vary depending on the type of cellular host. For example, calcium chloride transfection is commonly utilized for prokaryotic cells, whereas calcium phosphate treatment or electroporation may be used for other cellular hosts; see Sambrook, supra.

Once expressed, the chimeric fusion molecules of the present invention can be purified according to standard procedures of the art, including ammonium sulfate precipitation, affinity columns, column chromatography, gel electrophoresis and the like; see, Scopes, “Protein Purification”, Springer-Verlag, N.Y. (1982). In alternative aspects, the invention is directed to substantially pure chimeric polypeptides of at least about 90% to about 95% homogeneity; between about 95% to 98% homogeneity; and about 98% to about 99% or more homogeneity; these “substantially pure” polypeptides can be used in the preparation of pharmaceuticals. Once purified, partially or to a homogeneity as desired, the polypeptides may then be used therapeutically (including extracorporeally) or in developing and performing assay procedures.

In a still further embodiment, the present invention relates to a cell containing the polynucleotide or vector of the invention, or to a host cell transformed with a polynucleotide or vector of the invention. In alternative aspects, the host/cell is a eukaryotic cell, such as a mammalian cell, particularly if therapeutic uses of the polypeptide are envisaged. Of course, yeast and prokaryotic, e.g., bacterial cells, may serve as well, in particular, if the produced polypeptide is used for non-pharmaceutical purposes, e.g., as in diagnostic tests or kits or in screening methods.

The polynucleotide or vector of the invention that is present in the host cell may either be integrated into the genome of the host cell or it may be maintained extrachromosomally, e.g., as an episome.

The term “prokaryotic” is meant to include all bacteria that can be transformed or transfected with a DNA or RNA molecules for the expression of a polypeptide of the invention. Prokaryotic hosts may include gram negative as well as gram positive bacteria such as, for example, E. coli, S. typhimurium, Serratia marcescens and Bacillus subtilis. The term “eukaryotic” is meant to include yeast, higher plant, insect and mammalian cells. Depending upon the host employed in a recombinant production procedure, the chimeric fusion molecules of the present invention may be glycosylated or may be non-glycosylated. Chimeric fusion molecules of the invention may also include an initial methionine amino acid residue. A polynucleotide coding for a polypeptide of the invention can be used to transform or transfect the host using any of the techniques commonly known to those of ordinary skill in the art.

In one aspect, the nucleic acids encoding the chimeric polypeptide of the invention (including those sequences in vectors, e.g., plasmid or virus) further comprise, genetically fused thereto, sequences encoding an epitope tag, e.g., an N-terminal FLAG-tag and/or a C-terminal His-tag. In one aspect, the length of the FLAG-tag is about 4 to 8 amino acids; or, is about 8 amino acids in length. Methods for preparing fused, operably linked genes and expressing them in, e.g., mammalian cells and bacteria are well-known in the art (Sambrook, Molecular Cloning: A Laboratory Manual, Cold Spring Harbor Laboratory, Cold Spring Harbor, N.Y., 1989). The genetic constructs and methods described therein can be utilized for expression of the polypeptide of the invention in eukaryotic or prokaryotic hosts. In general, expression vectors containing promoter sequences which facilitate the efficient transcription of the inserted polynucleotide are used in connection with the host. The expression vector typically contains an origin of replication, a promoter, and a terminator, as well as specific genes which are capable of providing phenotypic selection of the transformed cells. Furthermore, transgenic non-human animals, such as mammals (e.g., mice, goats), comprising nucleic acids or cells of the invention may be used for the large scale production of the chimeric polypeptides of the invention.

In a further embodiment, the invention is directed to a process for the preparation of a polypeptide of the invention comprising cultivating a (host) cell of the invention under conditions suitable for the expression of the chimeric fusion molecule construct and isolating the polypeptide from the cell or the culture medium. The transformed hosts can be grown in fermentors and cultured according to techniques known in the art to achieve optimal cell growth. The produced constructs of the invention can then be isolated from the growth medium, cellular lysates, or cellular membrane fractions. The isolation and purification of the expressed polypeptides of the invention (e.g., microbially expressed) may be by any conventional means such as, e.g., preparative chromatographic separations and immunological separations, such as those involving the use of monoclonal or polyclonal antibodies directed against, e.g., a tag of the polypeptide of the invention or as described in the appended examples.

Depending on the host cell, renaturation techniques may be required to attain proper conformation. If necessary, point substitutions seeking to optimize binding may be made in the DNA using conventional cassette mutagenesis or other protein engineering methodology such as is disclosed herein. Preparation of the polypeptides of the invention may also be dependent on knowledge of the amino acid sequence (or corresponding DNA or RNA sequence) of bioactive proteins such as enzymes, toxins, growth factors, cell differentiation factors, receptors, anti-metabolites, hormones or various cytokines or lymphokines. Such sequences are reported in the literature and available through computerized data banks. The present invention further relates to a chimeric polypeptide, encoded by a polynucleotide of the invention or produced by the method described hereinabove.

Compositions

Additionally, the present invention provides for compositions comprising the polynucleotide, the vector, the host cell, and a chimeric fusion molecule, as described herein.

The term “composition”, in context of this invention, comprises at least one polynucleotide, vector, host cell, chimeric polypeptide of the invention, as described herein. The composition, optionally, further comprises other molecules, either alone or in combination, such as molecules which are capable of modulating and/or interfering with the immune system. The composition may be in solid, liquid or gaseous form and may be, inter alia, in a form of a powder(s), a tablet(s), a solution(s) or an aerosol(s). In alternative embodiments, the composition comprises at least two, at least three, at least four, or more than four, compounds of the invention. The composition can be a pharmaceutical composition further comprising, optionally, a pharmaceutically acceptable carrier, diluent and/or excipient.

Examples of suitable pharmaceutical carriers are well known in the art and include phosphate buffered saline solutions, water, emulsions, such as oil/water emulsions, various types of wetting agents, sterile solutions, etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical, intra-articular (including into or near the joint space) or intradermal administration. The dosage regiment can be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. Generally, the regimen as a regular administration of the pharmaceutical composition should be in the range of about 1 μg to 10 mg units per day. If the regimen is a continuous infusion, it can also be in the range of about 1 μg to 10 mg units per kilogram of body weight per minute, respectively. An alternative dosage for continuous infusion may be in the range of about 0.01 μg to 10 mg units per kilogram of body weight per hour. Other exemplary dosages are recited herein below. Progress can be monitored by periodic assessment.

The compositions of the invention may be administered locally or systematically. Administration can be parenterally, e.g., intravenously; and, by external administration. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like. Preservatives and other additives may also be present such as, for example, antimicrobials, anti-oxidants, chelating agents, and inert gases and the like. In addition, the pharmaceutical composition of the present invention may comprise proteinaceous carriers, like, e.g., serum albumin or immunoglobulin, including those of human origin. Furthermore, it is envisaged that the pharmaceutical composition of the invention may comprise further biologically active agents, depending on the intended use of the pharmaceutical composition. Such agents might be drugs acting on the immunological system, drugs used in tumor treatment.

Humanized Antibodies

In an preferred embodiment, antibodies of the invention comprise humanized antibodies. Humanized antibodies are antibodies whose light and heavy chain genes have been constructed, typically by genetic engineering, from immunoglobulin variable and constant region genes belonging to different species. For example, the variable segments of the genes from a mouse monoclonal antibody may be joined to human constant segments, such as gamma 1 and gamma 3. A typical therapeutic chimeric antibody is thus a hybrid protein composed of the variable or antigen-binding domain from a mouse antibody and the constant or effector domain from a human antibody, although other mammalian species may be used.

As used herein, the term “humanized” immunoglobulin refers to an immunoglobulin comprising a human framework region and one or more CDR's from a non-human (usually a mouse or rat) immunoglobulin. The non-human immunoglobulin providing the CDR's is called the “donor” and the human immunoglobulin providing the framework is called the “acceptor.” Constant regions need not be present, but if they are, they must be substantially identical to human immunoglobulin constant regions, i.e., at least about 85-90%, preferably about 95% or more identical. Hence, all parts of a humanized immunoglobulin, except possibly the CDR's, are substantially identical to corresponding parts of natural human immunoglobulin sequences. A “humanized antibody” is an antibody comprising a humanized light chain and a humanized heavy chain immunoglobulin, e.g., the entire variable region of a chimeric antibody is non-human. One says that the donor antibody has been “humanized”, by the process of “humanization”, because the resultant humanized antibody is expected to bind to the same antigen as the donor antibody that provides the CDR's.

It is understood that the humanized antibodies may have additional conservative amino acid substitutions which have substantially no effect on antigen binding or other immunoglobulin functions. By conservative substitutions are intended combinations such as gly, ala; val, ile, leu; asp, glu; asn, gln; ser, thr; lys, arg; and phe, tyr.

Humanized immunoglobulins, including humanized antibodies, have been constructed by means of genetic engineering. Most humanized immunoglobulins that have been previously described have comprised a framework that is identical to the framework of a particular human immunoglobulin chain, the acceptor, and three CDR's from a non-human donor immunoglobulin chain.

A principle is that as acceptor, a framework is used from a particular human immunoglobulin that is unusually homologous to the donor immunoglobulin to be humanized, or use a consensus framework from many human antibodies. For example, comparison of the sequence of a mouse heavy (or light) chain variable region against human heavy (or light) variable regions in a data bank (for example, the National Biomedical Research Foundation Protein Identification Resource) shows that the extent of homology to different human regions varies greatly, typically from about 40% to about 60-70%. By choosing as the acceptor immunoglobulin one of the human heavy (respectively light) chain variable regions that is most homologous to the heavy (respectively light) chain variable region of the donor immunoglobulin, fewer amino acids will be changed in going from the donor immunoglobulin to the humanized immunoglobulin. Hence, and again without intending to be bound by theory, it is believed that there is a smaller chance of changing an amino acid near the CDR's that distorts their conformation. Moreover, the precise overall shape of a humanized antibody comprising the humanized immunoglobulin chain may more closely resemble the shape of the donor antibody, also reducing the chance of distorting the CDR's.

Typically, one of the 3-5 most homologous heavy chain variable region sequences in a representative collection of at least about 10 to 20 distinct human heavy chains will be chosen as acceptor to provide the heavy chain framework, and similarly for the light chain. Preferably, one of the 1-3 most homologous variable regions will be used. The selected acceptor immunoglobulin chain will most preferably have at least about 65% homology in the framework region to the donor immunoglobulin.

In many cases, it may be considered preferable to use light and heavy chains from the same human antibody as acceptor sequences, to be sure the humanized light and heavy chains will make favorable contacts with each other. Regardless of how the acceptor immunoglobulin is chosen, higher affinity may be achieved by selecting a small number of amino acids in the framework of the humanized immunoglobulin chain to be the same as the amino acids at those positions in the donor rather than in the acceptor.

Humanized antibodies generally have advantages over mouse or in some cases chimeric antibodies for use in human therapy: because the effector portion is human, it may interact better with the other parts of the human immune system (e.g., destroy the target cells more efficiently by complement-dependent cytotoxicity (CDC) or antibody-dependent cellular cytotoxicity (ADCC)); the human immune system should not recognize the framework or constant region of the humanized antibody as foreign, and therefore the antibody response against such an antibody should be less than against a totally foreign mouse antibody or a partially foreign chimeric antibody.

Antibodies can also be genetically engineered. Particularly preferred are humanized immunoglobulins that are produced by expressing recombinant DNA segments encoding the heavy and light chain CDR's from a donor immunoglobulin capable of binding to a desired antigen, such as the tumor antigens e.g. HER2, attached to DNA segments encoding acceptor human framework regions.

The DNA segments typically further include an expression control DNA sequence operably linked to the humanized immunoglobulin coding sequences, including naturally-associated or heterologous promoter regions. Preferably, the expression control sequences will be eukaryotic promoter systems in vectors capable of transforming or transfecting eukaryotic host cells, but control sequences for prokaryotic hosts may also be used. Once the vector has been incorporated into the appropriate host, the host is maintained under conditions suitable for high level expression of the nucleotide sequences, and, as desired, the collection and purification of the humanized light chains, heavy chains, light/heavy chain dimers or intact antibodies, binding fragments or other immunoglobulin forms may follow (see, S. Beychok, Cells of Immunoglobulin Synthesis, Academic Press, New York, (1979), which is incorporated herein by reference).

Human constant region DNA sequences can be isolated in accordance with well known procedures from a variety of human cells, but preferably immortalized B-cells (see, Kabat op. cit. and WP87/02671). The CDR's for producing preferred immunoglobulins of the present invention will be similarly derived from monoclonal antibodies capable of binding to the predetermined antigen, such as the human T cell receptor CD3 complex, and produced by well known methods in any convenient mammalian source including, mice, rats, rabbits, or other vertebrates, capable of producing antibodies. Suitable source cells for the constant region and framework DNA sequences, and host cells for immunoglobulin expression and secretion, can be obtained from a number of sources, such as the American Type Culture Collection (“Catalogue of Cell Lines and Hybridomas,” sixth edition (1988) Rockville, Md., U.S.A., which is incorporated herein by reference).

Other “substantially homologous” modified immunoglobulins to the native sequences can be readily designed and manufactured utilizing various recombinant DNA techniques well known to those skilled in the art. For example, the framework regions can vary at the primary structure level by several amino acid substitutions, terminal and intermediate additions and deletions, and the like. Moreover, a variety of different human framework regions may be used singly or in combination as a basis for the humanized immunoglobulins of the present invention. In general, modifications of the genes may be readily accomplished by a variety of well-known techniques, such as site-directed mutagenesis (see, Gillman and Smith, Gene, 8, 81-97 (1979) and S. Roberts et al., Nature, 328, 731-734 (1987), both of which are incorporated herein by reference).

Substantially homologous immunoglobulin sequences are those which exhibit at least about 85% homology, usually at least about 90%, and preferably at least about 95% homology with a reference immunoglobulin protein.

Alternatively, polypeptide fragments comprising only a portion of the primary antibody structure may be produced, which fragments possess one or more immunoglobulin activities (e.g., complement fixation activity). These polypeptide fragments may be produced by proteolytic cleavage of intact antibodies by methods well known in the art, or by inserting stop codons at the desired locations in vectors known to those skilled in the art, using site-directed mutagenesis.

As stated previously, the DNA sequences can be expressed in hosts after the sequences have been operably linked to (i.e., positioned to ensure the functioning of) an expression control sequence. These expression vectors are typically replicable in the host organisms either as episomes or as an integral part of the host chromosomal DNA. Commonly, expression vectors contain selection markers, e.g., tetracycline or neomycin resistance, to permit detection of those cells transformed with the desired DNA sequences (see, e.g., U.S. Pat. No. 4,704,362, which is incorporated herein by reference).

E. coli is one prokaryotic host useful particularly for cloning the DNA sequences of the present invention. Other microbial hosts suitable for use include bacilli, such as Bacillus subtilus, and other enterobacteriaceae, such as Salmonella, Serratia, and various Pseudomonasspecies. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters will typically control expression, optionally with an operator sequence, and have ribosome binding site sequences and the like, for initiating and completing transcription and translation.

Other microbes, such as yeast, may also be used for expression. Saccharomyces is a preferred host, with suitable vectors having expression control sequences, such as promoters, including 3-phosphoglycerate kinase or other glycolytic enzymes, and an origin of replication, termination sequences and the like as desired.

In addition to microorganisms, mammalian tissue cell culture may also be used to express and produce the polypeptides of the present invention (see, Winnacker, “From Genes to Clones,” VCH Publishers, New York, N.Y. (1987), which is incorporated herein by reference). Eukaryotic cells are actually preferred, because a number of suitable host cell lines capable of secreting intact immunoglobulins have been developed in the art, and include the CHO cell lines, various COS cell lines, HeLa cells, preferably myeloma cell lines, etc, and transformed B-cells or hybridomas. Expression vectors for these cells can include expression control sequences, such as an origin of replication, a promoter, an enhancer (Queen et al., Immunol. Rev., 89, 49-68 (1986), which is incorporated herein by reference), and necessary processing information sites, such as ribosome binding sites, RNA splice sites, polyadenylation sites, and transcriptional terminator sequences. Preferred expression control sequences are promoters derived from immunoglobulin genes, SV40, Adenovirus, cytomegalovirus, Bovine Papilloma Virus, and the like.

The vectors containing the DNA segments of interest (e.g., the heavy and light chain encoding sequences and expression control sequences) can be transferred into the host cell by well-known methods, which vary depending on the type of cellular host. For example, calcium chloride transfection is commonly utilized for prokaryotic cells, whereas calcium phosphate treatment or electroporation may be used for other cellular hosts. (See, generally, Maniatis et al., Molecular Cloning: A Laboratory Manual, Cold Spring Harbor Press, (1982), which is incorporated herein by reference.)

Once expressed, the whole antibodies, their dimers, individual light and heavy chains, or other immunoglobulin forms of the present invention, can be purified according to standard procedures of the art, including ammonium sulfate precipitation, affinity columns, column chromatography, gel electrophoresis and the like (see, generally, R. Scopes, “Protein Purification”, Springer-Verlag, N.Y. (1982)). Substantially pure immunoglobulins of at least about 90 to 95% homogeneity are preferred, and 98 to 99% or more homogeneity most preferred, for pharmaceutical uses. Once purified, partially or to homogeneity as desired, the polypeptides may then be used therapeutically (including extracorporeally) or in developing and performing assay procedures, immunofluorescent staining, and the like. (See, generally, Immunological Methods, Vols. I and II, Lefkovits and Pernis, eds., Academic Press, New York, N.Y. (1979 and 1981)).

In general, the subject humanized antibodies are produced by obtaining nucleic acid sequences encoding the variable heavy and variable light sequences of an antibody which binds a tumor antigen, preferably HER2/neu, identifying the CDRs in the variable heavy and variable light sequences, and grafting such CDR nucleic acid sequences onto human framework nucleic acid sequences.

Preferably, the selected human framework will be one that is expected to be suitable for in vivo administration, i.e., does not exhibit immunogenicity. This can be determined, e.g., by prior experience with in vivo usage of such antibodies and by studies of amino acid sequence similarities. In the latter approach, the amino acid sequences of the framework regions of the antibody to be humanized, will be compared to those of known human framework regions, and human framework regions used for CDR grafting will be selected which comprise a size and sequence most similar to that of the parent antibody, e.g., a murine antibody which binds HER2/neu. Numerous human framework regions have been isolated and their sequences reported in the literature. See, e.g., Kabat et al., (id.). This enhances the likelihood that the resultant CDR-grafted “humanized” antibody, which contains the CDRs of the parent (e.g., murine) antibody grafted onto the selected human framework regions will significantly retain the antigen binding structure and thus the binding affinity of the parent antibody.

Methods for cloning nucleic acid sequences encoding immunoglobulins are well known in the art and are described in detail in the Examples which follow. Such methods will generally involve the amplification of the immunoglobulin sequences to be cloned using appropriate primers by polymerase chain reaction (PCR). Primers suitable for amplifying immunoglobulin nucleic acid sequences, and specifically murine variable heavy and variable light sequences have been reported in the literature. After such immunoglobulin sequences have been cloned, they will be sequenced by methods well known in the art. This will be effected in order to identify the variable heavy and variable light sequences, and more specifically the portions thereof which constitute the CDRs and FRs. This can be effected by well known methods.

Once the CDRs and FRs of the cloned antibody sequences which are to be humanized have been identified, the amino acid sequences encoding CDRs are then identified (deduced based on the nucleic acid sequences and the genetic code and by comparison to previous antibody sequences) and the corresponding nucleic acid sequences are grafted onto selected human FRs. This may be accomplished by use of appropriate primers and linkers. Methods for selecting suitable primers and linkers to provide for ligation of desired nucleic acid sequences is well within the purview of the ordinary artisan and include those disclosed in U.S. Pat. No. 4,816,397 to Boss et al. and U.S. Pat. No. 5,225,539 to Winter et al.

After the CDRs are grafted onto selected human FRs, the resultant “humanized” variable heavy and variable light sequences will then be expressed to produce a humanized chimeric fusion molecule which binds, for example, HER2/neu. The humanized variable heavy and/or variable light sequences will be expressed as a fusion protein so that an intact chimeric fusion molecule which binds, for example, HER2/neu is produced.

In another preferred embodiment, the variable heavy and light sequences can also be expressed in the absence of constant sequences to produce a humanized Fv chimeric fusion molecule which binds, for example, HER2/neu. However, fusion of human constant sequences to the humanized variable region(s) is potentially desirable because the resultant humanized antibody which binds, for example, HER2/neu will then possess human effector functions such as complement-dependent cytotoxicity (CDC) and antibody-dependent cellular cytotoxicity (ADCC) activity.

The following references are representative of methods and vectors suitable for expression of recombinant immunoglobulins which may be utilized in carrying out the present invention. Weidle et al., Gene, 51:21-29 (1987); Dorai et al., J. Immunol., 13(12):4232-4241 (1987); De Waele et al., Eur. J. Biochem., 176:287-295 (1988); Colcher et al., Cancer Res., 49:1738-1745 (1989); Wood et al., J. Immunol., 145(a):3011-3016 (1990); Bulens et al., Eur. J. Biochem., 195:235-242 (1991); Beggington et al., Biol. Technology, 10:169 (1992); King et al., Biochem. J., 281:317-323 (1992); Page et al., Biol. Technology, 9:64 (1991); King et al., Biochem. J., 290:723-729 (1993); Chaudary et al., Nature, 339:394-397 (1989); Jones et al., Nature, 321:522-525 (1986); Morrison and Oi, Adv. Immunol, 44:65-92 (1988); Benhar et al., Proc. Natl. Acad. Sci. USA, 91:12051-12055 (1994); Singer et al., J. Immunol., 150:2844-2857 (1993); Cooto et al., Hybridoma, 13(3):215-219 (1994); Queen et al., Proc. Natl. Acad. Sci. USA, 86:10029-10033 (1989); Caron et al., Cancer Res., 32:6761-6767 (1992); Cotoma et al., J. Immunol. Meth., 152:89-109 (1992). Moreover, vectors suitable for expression of recombinant antibodies are commercially available. The vector may, e.g., be a bare nucleic acid segment, a carrier-associated nucleic acid segment, a nucleoprotein, a plasmid, a virus, a viroid, or a transposable element.

Host cells known to be capable of expressing functional immunoglobulins include, e.g.: mammalian cells such as Chinese Hamster Ovary (CHO) cells; COS cells; myeloma cells, such as NSO and SP2/0 cells; bacteria such as Escherichia coli; yeast cells such as Saccharomyces cerevisiae; and other host cells. SP2/0 cells are one of the preferred types of host cells useful in the present invention.

After expression, the antigen binding affinity of the resulting humanized antibody will be assayed by known methods, e.g., Scatchard analysis. In a particularly preferred embodiment, the antigen-binding affinity of the humanized antibody will be at least 50% of that of the parent antibody, e.g., anti-HER2/neu, more preferably, the affinity of the humanized antibody will be at least about 75% of that of the parent antibody, more preferably, the affinity of the humanized antibody will be at least about 100%, 150%, 200% or 500% of that of the parent antibody.

In some instances, humanized antibodies produced by grafting CDRs (from an antibody which binds, for example, a tumor antigen such as, for example, HER/neu) onto selected human framework regions may provide humanized antibodies having the desired affinity to HER2/neu. However, it may be necessary or desirable to further modify specific residues of the selected human framework in order to enhance antigen binding. This may occur because it is believed that some framework residues are essential to or at least affect antigen binding. Preferably, those framework residues of the parent (e.g., murine) antibody which maintain or affect combining-site structures will be retained. These residues may be identified by X-ray crystallography of the parent antibody or Fab fragment, thereby identifying the three-dimensional structure of the antigen-binding site. Also, framework residues involved in antigen binding may potentially be identified based on previously reported humanized murine antibody sequences. Thus, it may be beneficial to retain such framework residues or others from the parent murine antibody to optimize, for example, HER2/neu binding. Preferably, such methodology will confer a “human-like” character to the resultant humanized antibody thus rendering it less immunogenic while retaining the interior and contacting residues which affect antigen-binding.

The present invention further embraces variants and equivalents which are substantially homologous to the humanized antibodies and antibody fragments set forth herein. These may contain, e.g., conservative substitution mutations, i.e. the substitution of one or more amino acids by similar amino acids. For example, conservative substitution refers to the substitution of an amino acid with another within the same general class, e.g., one acidic amino acid with another acidic amino acid, one basic amino acid with another basic amino acid, or one neutral amino acid by another neutral amino acid. What is intended by a conservative amino acid substitution is well known in the art.

Methods of Delivering a Chimeric Molecule to a Cell

The invention also provides a method of delivering an anti-angiogenic agent-carrier chimeric molecule to a cell. The chimeric molecules of the invention can be delivered to a cell by any known method. For example, a composition containing the chimeric molecule can be added to cells suspended in medium. Alternatively, a chimeric molecule can be administered to an animal (e.g., by a parenteral route) having a cell expressing a receptor that binds the chimeric molecule so that the chimeric molecule binds to the cell in situ. For example, the chimeric molecules of this invention that feature an Ig domain that is specific for HER2/neu are particularly well suited as targeting moieties for binding tumor cells that overexpress HER2/neu, e.g., breast cancer and ovarian cancer cells.

Administration of Compositions to Animals

For targeting a tumor cell in situ, the compositions described above may be administered to animals including human beings in any suitable formulation. For example, compositions for targeting a tumor cell may be formulated in pharmaceutically acceptable carriers or diluents such as physiological saline or a buffered salt solution. Suitable carriers and diluents can be selected on the basis of mode and route of administration and standard pharmaceutical practice. A description of exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

The compositions of the invention may be administered to animals by any conventional technique. The compositions may be administered directly to a target site by, for example, surgical delivery to an internal or external target site, or by catheter to a site accessible by a blood vessel. Other methods of delivery, e.g., liposomal delivery or diffusion from a device impregnated with the composition, are known in the art. The compositions may be administered in a single bolus, multiple injections, or by continuous infusion (e.g., intravenously). For parenteral administration, the compositions are preferably formulated in a sterilized pyrogen-free form.

Formulations

While it is possible for an antibody or fragment thereof to be administered alone, it is preferable to present it as a pharmaceutical formulation. The active ingredient may comprise, for topical administration, from 0.001% to 10% w/w, e.g., from 1% to 2% by weight of the formulation, although it may comprise as much as 10% w/w but preferably not in excess of 5% w/w and more preferably from 0.1% to 1% w/w of the formulation. The topical formulations of the present invention, comprise an active ingredient together with one or more acceptable carrier(s) therefor and optionally any other therapeutic ingredients(s). The carrier(s) must be “acceptable” in the sense of being compatible with the other ingredients of the formulation and not deleterious to the recipient thereof.

Formulations suitable for topical administration include liquid or semi-liquid preparations suitable for penetration through the skin to the site of where treatment is required, such as liniments, lotions, creams, ointments or pastes, and drops suitable for administration to the eye, ear, or nose. Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified and sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogels. The formulation may incorporate any suitable surface active agent such as an anionic, cationic or non-ionic surface active such as sorbitan esters or polyoxyethylene derivatives thereof. Suspending agents such as natural gums, cellulose derivatives or inorganic materials such as silicaceous silicas, and other ingredients such as lanolin, may also be included.

Kits

Kits according to the present invention include frozen or lyophilized humanized antibodies or humanized antibody fragments to be reconstituted, respectively, by thawing (optionally followed by further dilution) or by suspension in a (preferably buffered) liquid vehicle. The kits may also include buffer and/or excipient solutions (in liquid or frozen form)—or buffer and/or excipient powder preparations to be reconstituted with water—for the purpose of mixing with the humanized antibodies or humanized antibody fragments to produce a formulation suitable for administration. Thus, preferably the kits containing the humanized antibodies or humanized antibody fragments are frozen, lyophilized, pre-diluted, or pre-mixed at such a concentration that the addition of a predetermined amount of heat, of water, or of a solution provided in the kit will result in a formulation of sufficient concentration and pH as to be effective for in vivo or in vitro use in the treatment or diagnosis of cancer. Preferably, such a kit will also comprise instructions for reconstituting and using the humanized antibody or humanized antibody fragment composition to treat or detect cancer. The kit may also comprise two or more component parts for the reconstituted active composition. For example, a second component part—in addition to the humanized antibodies or humanized antibody fragments—may be bifunctional chelant, bifunctional chelate, or a therapeutic agent such as a radionuclide, which when mixed with the humanized antibodies or humanized antibody fragments forms a conjugated system therewith. The above-noted buffers, excipients, and other component parts can be sold separately or together with the kit.

It will be recognized by one of skill in the art that the optimal quantity and spacing of individual dosages of a humanized antibody or humanized antibody fragment of the invention will be determined by the nature and extent of the condition being treated, the form, route and site of administration, and the particular animal being treated, and that such optima can be determined by conventional techniques. It will also be appreciated by one of skill in the art that the optimal course of treatment, i.e., the number of doses of an antibody or fragment thereof of the invention given per day for a defined number of days, can be ascertained by those skilled in the art using conventional course of treatment determination tests.

Anti-Cancer and Chimeric Fusion Molecule Cocktails

The subject chimeric fusion molecules, including the humanized chimeric fusion molecules may also be administered in combination with other anti-cancer agents, e.g., other antibodies or drugs. Also, the subject chimeric molecules or fragments may be directly or indirectly attached to effector having therapeutic activity. Suitable effector moieties include by way of example cytokines (IL-2, TNF, interferons, colony stimulating factors, IL-1, etc.), cytotoxins (Pseudomonasexotoxin, ricin, abrin, etc.), radionuclides, such as ⁹⁰Y, ¹³¹I, ¹¹¹In, 125I, among others, drugs (methotrexate, daunorubicin, doxorubicin, etc.), immunomodulators, therapeutic enzymes (e.g., beta-galactosidase), anti-proliferative agents, etc. The attachment of antibodies to desired effectors is well known. See, e.g., U.S. Pat. No. 5,435,990 to Cheng et al. Moreover, bifunctional linkers for facilitating such attachment are well known and widely available. Also, chelators (chelants and chelates) providing for attachment of radionuclides are well known and available.

The subject chimeric fusion molecules may be used alone or in combination with other antibodies, e.g. anti-HER2/neu.

Without further elaboration, it is believed that one skilled in the art can, using the preceding description, utilize the present invention to its fullest extent. The invention will be further clarified by a consideration of the following examples, which are intended to be purely exemplary of the present invention are thus to be construed as merely illustrative examples and not limitations of the scope of the present invention in any way.

### EXAMPLES

The present invention is further illustrated by the following specific examples. The examples are provided for illustration only and are not to be construed as limiting the scope or content of the invention in any way.

Materials & Methods

Cell Lines and Animals

CT26, CT26-HER2, human embryonic kidney (HEK) 293, and transfected Sp2/0 cells were cultured in Iscove's Modified Dulbecco's Medium (IMDM; Cellgro, Mediatech, Inc., Herndon, Va.) with 5% calf serum (GIBCO, Invitrogen Corp. Carlsbad, Calif.). Female BALB/c mice (4-6 weeks) and SCID mice (4-6 weeks) were purchased from the Jackson Laboratory (Bar Harbor, Me.). Animal care and use procedures were performed in accordance with standards described in the National Institutes of Health Guide for Care and Use of Laboratory Animals.

Construction, Expression, and Characterization of Anti-HER2/neu IgG3-Endostatin Fusion Protein

Experimental murine endostatin gene originated from pFLAG-CMV-1-endostatin by PCR using primers 5′-CCCCTCGCGATATCATACTCATCAGGACTTTCAGCC-3′ (SEQ ID NO 1) and 5′-CCCCGAATTCGTTAACCTTTGGAGAAAGAGGTCATGAAGC-3′ (SEQ ID NO 2). PCR products were subcloned into p-GEM-T Easy Vector (Promega, Madison, Wis.), then sequenced for verification. The EcORV-EcOR1 fragment of the subcloned endostatin gene was ligated to the carboxyl end of the heavy chain constant domain (C_{H}3) of human IgG3 in the vector pAT135, as previously described (Shin S U et al., J Immunol. 1997;158(10):4797-804.). To complete the construct, the IgG3-endostatin heavy chain constant region (Agel-BamHI) was then joined to an anti-HER2/neu variable region of a recombinant humanized monoclonal antibody 4D5-8 (rhuMAb HER2, Herceptin; Genentech, San Francisco, Calif.) in the expression vector (pSV2-his) containing HisD gene for eukaryotic selection (Challita-Eid P M. et al., J Immunol 1998;160(7):3419-26; Coloma M J. et al, J. Immunol. Methods 1992;152:89-104). The finished anti-HER2/neu heavy chain IgG3-endostatin construction vector was transfected by electroporation into Sp2/0 cells stably expressing the anti-HER2/neu K light chain in order to assemble entire anti-HER2/neu IgG3-endostatin fusion proteins. Transfected cells were selected with 5 mM histidinol and transfectomas producing the fusion proteins were identified by a enzyme-linked immunosorbent assay (ELISA) using anti-human IgG antibody coated plates and an anti-human kappa detection antibody (Sigma, Saint Louis, Mo.). The anti-HER2/neu IgG3-endostatin fusion proteins were biosynthetically labeled with [³⁵S]methionine (Amersham Biosciences, Piscataway, N.J.) and analyzed by SDS-PAGE on 5% sodium phosphate buffered polyacrylamide gels without reduction or on 12.5% Tris-glycine buffered polyacrylamide gels following treatment with 0.15 M β-mercaptoethanol at 37° C. for 30 min. The fusion protein was purified from culture supernatants using protein A immobilized on Sepharose 4B fast flow (Sigma, Saint Louis, Mo.).

To obtain active endostatin, a mouse endostatin expression vector (pFLAG-CMV-1-endostatin) was co-transfected with pcDNA3.1 (CLONTECH, Palo Alto, Calif.) into human embryonic kidney (HEK) 293 cells, and G418 (0.6 μg/ml)-resistant cells selected. Secreted endostatin was harvested from serum-free conditioned medium and purified in a heparin-Sepharose CL-6B column. Purity was assessed by Coomassie blue staining of the SDS-PAGE gels. For Western blot analysis, the endostatin fusion proteins were treated with β-mercaptoethanol, fractionated by SDS-PAGE and transferred onto a membrane. Rabbit anti-endostatin (BodyTech, Kangwon-Do, Korea) was used as the primary antibody and mouse anti-rabbit IgG conjugated with HRP (Sigma, St. Louis, Mo.) was used as the secondary antibody. Goat anti-human IgG conjugated with HRP (Sigma, Saint Louis, Mo.) was used to detect human antibody.

Chorioallantoic Membrane (CAM) Assay

The ability of anti-HER2/neu IgG3-endostatin to block VEGF/bFGF-induced angiogenesis was tested by CAM assay, which employed Leghorn chicken embryos (Charles River SPAFAS, Wilmington, Mass.) at 12-14 days of embryonic development. Vitrogen gel pellets (Collagen Biomaterials, Palo Alto, Calif.) were supplemented with (a) vehicle (0.1% DMSO) in PBS alone (negative control); (b) VEGF/bFGF (100 ng and 50 ng/pellet, respectively; positive control); or (c) VEGF/bFGF and either of anti-HER2/neu IgG3, anti-HER2/neu IgG3-endostatin, or endostatin, at various concentrations (0.5-10 μg/pellet) and were allowed to polymerize at 37° C. for 2 h. Pellets were then placed on a nylon mesh (pore size 250 μm; Tetko Inc., USA) and polymerized mesh was placed onto the outer region of the chorioallantoic membrane of the embryo and incubated for 24 hours as described (Iruela-Arispe M L, et al., Thromb. Haemost. 1997;78(1):672-7; Iruela-Arispe M L, et al., Circulation 1999; 100(13):1423-31.). To visualize vessels, 400 μl of fluorescein isothiocyanate-dextran (100 μg/ml, Sigma, USA) was injected in the chick embryo blood stream. After 5-10 min of incubation, the chick embryo was topically fixed with 3.7% formaldehyde for 5 min. The implanted mesh was then dissected and mounted on slides. Fluorescence intensity was analyzed with a computer-assisted image program (NIH Image 1.59).

Pharmacokinetic and Biodistribution of Anti-HER2/neu IgG3-Endostatin

Anti-HER2/neu IgG3 (100 μg), anti-HER2/neu IgG3-endostatin (100 μg), anti-dansyl IgG3 (100 μg), and endostatin (100 μg) were iodinated with 0.5 mCi of [¹²⁵,] (Amersham Biosciences, Piscataway, N.J.) by the chloramine T method (Pardridge W M, et al., Proc. Natl. Acad. Sci. USA 1995;92:5592.). BALB/c mice (4-6 weeks of age) were injected s.c. with either 1×10⁶ CT26-HER2 or CT26 cells or left uninjected. Groups of three mice with either CT26-HER2 or CT26 tumors or no tumor were injected i.v. with either 32 μCi of [¹²⁵I]-anti-HER2/neu IgG3, 30 μCi of [¹²⁵I]-anti-HER2/neu IgG3-endostatin, 32 μCi of [¹²⁵I]-anti-dansyl IgG3, or 24 μCi of [¹²⁵I]-endostatin. Blood samples were serially obtained at various intervals ranging from 15 min to 96 hours from the retro-orbital plexus of mice injected with either the anti-HER2/neu IgG3, anti-HER2/neu IgG3-endostatin, or anti-dansyl IgG3. Mice injected with endostatin alone were bled within 15 second to 60 min after the i.v. injection. The TCA precipitable radioactivity in each blood sample was measured in a γ-counter. The pharmacokinetic parameters were calculated by fitting plasma radioactivity data to a bi-exponential equation as described previously (Shin S U, et al., J Immunol. 1997; 158(10):4797-804., Yoshikawa T et al., J Pharmacol. Exp. Ther. 1992;263:897; Penichet M L. et al., J Immunol. 1999;163(8):4421-6.).
Cp(t)=Aₗe^{-K}1^{t}+A₂e^{-K}2^{t}

The equation was fit to plasma data using derivative free nonlinear regression analysis (PARBMDP, Biomedical Computer P series Program developed at UCLA Health Sciences Computing Facilities). Data were weighed using weight=1/(concentration)², where concentration was either count per minute (cpm) per microliter (μl) or % ID (percentage of injected dose) per milliliter. Area under the plasma concentration curve (AUC) and mean residence time (MRT) were calculated from the slopes and intercept of the bi-exponential equation. The volume of distribution (V_{D}) of the antibodies was determined from the ratio of disintegrations per minute per gram of organ divided by disintegrations per minute per microliters of corresponding plasma at each time after injection. The organ permeability-surface area product (Ki) of the antibodies was calculated from,
Ki=[V_{D}−V₀]Cp(T)/AUC(t)
where Cp(T) is the terminal plasma concentration and V₀ is the organ plasma volume. The organ delivery of the antibodies was determined from,
%ID/g=Ki×AUC(t)
where Ki and AUC(t) correspond to the 1, 48, or 96 hour time period after injection.

The pharmacokinetic parameters were calculated by fitting plasma TCA-precipitable radioactivity data to a bi-exponential equation as described previously (Shin S U, et al., J. Immunol. 1997; 158(10):4797-804., Yoshikawa T et al., J Pharmacol. Exp. Ther. 1992;263:897; Penichet M L. et al., J Immunol. 1999;163(8):4421-6; Gibaldi M. et al., Pharmacokinetics, Marcel Dekker, Inc., New York. 1982; Pardridge W M. et al., J. Pharm. Sci. 1995;84:943-8.). Plasma clearance, the initial plasma volume, systemic volume of distribution, steady state area under the plasma concentration curve (AUC_{0-∞}), and mean residence time were also determined.

Following the pharmacokinetic experiments, mice were exsanguinated by perfusion with 20 ml PBS for measurements of the tissue distribution of ¹²⁵I-labeled antibody-endostatin fusion protein. The heart, lung, liver, spleen, kidney, muscle, and tumor were removed, weighed, γ-counted and the percent of injected dose per gram of tissue calculated. Specific tumor targeting is expressed as the radiolocalization index (the % ID/g in tumor divided by the % ID/g in blood).

To determine the preferential distribution and localization of the ¹²⁵I-labeled proteins in mice simultaneously implanted with CT26 and CT26-HER2 tumors on opposite flanks, groups of three mice were injected i.v. with either 5 μCi [¹²⁵I]-anti-HER2/neu IgG3-endostatin fusion protein or 5 μCi [¹²⁵I]-anti-HER2/neu IgG3. The animals were sacrificed at different times (6, 24, and 96 hours) after injection of labeled protein and organs (e.g., lung, liver, kidney, spleen, muscle, CT26 tumor, CT26-HER2 tumor, blood, and urine) were isolated after perfusion of the mouse with PBS, weighed, and counted in a gamma scintillation counter. The percentage of injected dose per gram (% ID/g) for each organ was determined as above.

In Vivo Anti-Tumor Effects.

The in vivo anti-tumor efficacy of anti-HER2/neu IgG3-endostatin was examined using a CT26-HER2 BALB/c syngeneic mouse model and a SK-BR-3 human breast xenograft SCID mouse model.

To determine targeting and efficacy of anti-HER2/neu IgG3-endostatin, BALB/c (8/group, 4-6 weeks of age) mice were injected s.c. in the right flank with 1×10⁶ CT26-HER2 cells and control CT26 cells injected on the left flank. On day seven, mice (8 mice/group) were injected i.v. with the anti-HER2/neu IgG3-endostatin fusion proteins (42 μg/injection, 2×10⁻¹⁰ mole, equimolar to 8 μg of endostatin), anti-HER2/neu IgG3 alone (34 μg/injection, 2×10⁻¹⁰ mole), endostatin alone (8 μg/injection, 4×10⁻¹⁰ mole), the combination of anti-HER2/neu IgG3 (34 μg) and endostatin (8 μg), or PBS as a control. All mice received seven treatments at 2-day intervals. Tumor size and growth rates were recorded and calculated using the following equation:
Tumor Volume (mm³)=4/3×3.14×{(Long axis+Short axis)/4}³

Human breast cancer SK-BR-3 xenografts in SCID mice was also used to evaluate anti-tumor activity of anti-HER2/neu IgG3-endostatin fusion protein. SK-BR-3 (1×10⁶ cells per mouse) was implanted on the flank of SCID mice. On day 15, mice (8 mice/group) were injected i.v. with the anti-HER2/neu IgG3-endostatin fusion proteins (42 μg), anti-HER2/neu IgG3 (34 μg), the combination of anti-HER2/neu IgG3 (34 μg) and endostatin (8 μg), or endostatin (8 μg). This treatment was repeated every other day. Visible tumors were measured using a caliper and the tumor growth rate analyzed as described above.

Immunohistochemistry and Image Analysis of Blood Vessel Formation

Mice were killed at the end of the experiments. Tumors were placed in OCT Compound (Tissue-Tek, Elkhart, IN) and snap frozen in isopentane chilled with liquid nitrogen. Frozen tumors were stored at −80° C. until further use. For conventional immunohistochemistry, five-μm tissue sections were cut using a cryostat (Shandon, Pittsburgh, Pa.) and placed on positively charged slides (Fisher Scientific, Pittsburgh, Pa.). Tumor sections were air-dried and fixed with 4% paraformaldehyde for 10 min. To analyze the microvessel formation in tumors, sections were stained with a rat anti-mouse platelet-endothelial cell adhesion molecule 1 (PECAM-1; CD31) MAb (PharMingen, San Diego, Calif.) and subsequently with the ABC (Vector Lab, Burlingame, Calif.) method. HER2/neu expression on tumors has been examined with staining tumor sections with anti-HER2/neu IgG3-endostatin fusion antibody. All sections were counter-stained with hematoxylin (Sigma, St. Louis, Mo.). Positively stained vascular endothelial cells (brown) were visualized and imaged using a digital camera attached to a Zeiss microscope (Carl Zeiss, Thornwood, N.Y.).

For confocal microscopic analysis, thirty-μm cryosections were cut and stained with a rat anti-mouse CD31 Mab. Blood vessels have been visualized with anti-rat IgG-Alexa 594 (Molecular Probes, Eugene, Oreg.) and a coverslip was placed on top of the piece of sections with anti-fade mounting media (Vectorshield: Vector Lab, Burlingame, Calif.). These fluorescent blood vessels were then viewed via LSM5 confocal microscope (Carl Zeiss, Thornwood, N.Y.), and 14-21 digital images were obtained per section. These digital images have been composed as one image per each section to measure blood vessel density, and blood vessel area (pixel²) was then computed from the composite images and averaged to measure blood vessel density per tumor. Images were analyzed using NIH ImageJ v1.31 software by color image to form a binary image of the tumor blood vessels.

Statistical Analysis.

Antiangiogenic activity, pharmacokinetics, biodistribution, and tumor growth are presented as the mean±SEM. Two-sided Student's t test was used to determine the significance of differences between two group means. Differences were considered statistically significant at P<0.05. All statistical tests were two-sided.

Focus Formation Assay:

Focus formation assay is used to determine whether anti-HER2/neu antibody-endostatin fusions protein will exert antiproliferative effects on tumor bearing HER2/neu antigens. In vitro SK-BR-3, BT474, MCF7-HER2 (positive tumor cells) and MCF7 (negative tumor cell) are treated with different concentrations of anti-HER2/neu antibody-endostatin fusion proteins (0.1, 1, 10 μg/ml). One thousand of tumor cells are plated in 60-mm dishes in 1.5 ml of medium containing 0.33% agar, which are overlaid onto solidified 0.5% agar medium. The medium used for soft agar assays is DMEM containing 10% fetal calf serum, and contains the endostatin fusion proteins. The soft agar plates are fed with 0.5 ml of medium every 5-7 days, and after 14 days, the cells will be stained overnight (at 37° C. and 5% CO₂) with the vital dye p-iodonitrotetrazolium violet (Sigma), and counted. The resulting foci are stained with crystal violet and counted.

MTT Assay:

If tumor cells do not grow properly on soft agar assays, MTT assay are used to evaluate the antiproliferative effect of anti-HER2/neu antibody-endostatin fusion proteins on tumor expressing HER2/neu. Tumor cells such as SK-BR-3, BT474, MCF7 and MCF7-HER2, CT26 and CT-HER2/neu, or EMT6 and EMT6-HER2/neu are treated with different concentrations (0.1, 1, 10 μg/ml) of anti-HER2/neu antibody-endostatin fusion proteins or controls. Briefly, tumor cells will be plated out at 2-5×10³ cells/well on 96 well plates and allowed to adhere overnight. The following day tumor cells are treated with various concentrations of endostatin fusion proteins or control, and incubated for a further 48-72 hours. To determine cell growth, 20 pl of 10 mg/ml MTT (3-(4,4-dimethylthiazol-2-yl)-2,5-diphenyl tetrazolium bromide; Sigma) is added to each well and the plates are incubated at 37° C. in 5% CO₂ for a further three hours. The supernatant is removed and the formed crystals dissolved in 200 μl dimethyl sulphoxide. The plates are then quantitated by determining their absorbance at 595 to 600 nm in a microplate reader. Growth inhibition is calculated by expressing the differences in optical densities between treatment wells and control wells as a percentage of the control. Each assay is performed in triplicate.

Effect on VEGF Secretion:

The following cell lines are tested for effects of anti-HER2/neu IgG3 control and/or endostatin, and to testify informative cell lines that respond to endostatin, anti-HER2/neu antibody, or both proteins. To investigate effect of anti-HER2/neu antibody-endostatin fusion proteins on VEGF family expression, tumor cells such as SK-BR-3, BT474, MCF7 and MCF7-HER2, CT26 and CT-HER2/neu, or EMT6 and EMT6-HER2/neu are treated with anti-HER2/neu antibody-endostatin fusion proteins. 5×10⁵ tumor cells/well are seeded in 24-well plates (Falcon). Cells are allowed to adhere overnight, and then treated with different concentrations (0.1, 1, 10, 100 μg/ml) of endostatin fusion proteins, endostatin, or antibody. Cells are removed by centrifugation at different time points (24, 48, 96 hours), and the supernatants filtered using a 0.22-μm pore size filter. Secreted VEGF levels are analyzed by a sandwich ELISA (R&D Systems, Minneapolis, Minn., USA) that detects all VEGF spliced forms. Human recombinant VEGF165 (R&D Systems, Minneapolis, Minn., USA) serves as the standard.

Endothelial Cell Proliferation Assay:

The antiproliferative effect of anti-HER2/neu antibody-endostatin fusion proteins are tested using C-PAE cells. The cells are plated in 24-well fibronectin (10 μg/ml)-coated plates at 12,500 cells/well in 0.5 ml of DMEM containing 2% FBS. After a 24-h incubation at 37° C., the medium is replaced with fresh DMEM and 2% FBS containing 3 ng/ml of bFGF (R & D systems, Minneapolis, Minn., USA) with or without endostatin fusion proteins and endostatin (1, 10, or 100 μg/ml). The cells are pulsed with 1 μCi of [³H]thymidine for 24 h. Medium is aspirated, cells are washed three times with PBS, and then solubilized by addition of 1.5 N NaOH (100 μl/well) and incubated at 37° C. for 30 min. Cell-associated radioactivity is determined with a liquid scintillation counter.

Migration Assay:

To determine the ability of anti-HER2/neu antibody-endostatin fusion proteins to block migration of human endothelial cells (ECV304) toward bFGF, a migration assay is performed using 12-well Boyden chemotaxis chambers (Neuro Probe, Inc.) with a polycarbonate membrane (25×80-mm, PVD free, 8-μm pores; Poretics Corp., Livermore, Calif.). The nonspecific binding of growth factor to the chambers is prevented by coating the chambers with a solution containing 0.5% gelatin, 1 mM CaCl₂, and 150 mM NaCl at 37° C. overnight. ECV304 cells are grown in 10% FBS containing 5 ng/ml 1,1′-dioctadecyl-3,3,3′,3′-tetramethylindocarbocyanine perchlorate (DiIC18; Molecular Probes, Eugene, Oreg.) overnight and washed with PBS containing 0.5% BSA. After trypsinization, the cells are counted and diluted to 300,000 cells/ml in medium containing 0.5% FBS. The lower chamber is filled with medium containing 25 ng/ml bFGF. The upper chamber is seeded with 15,000 cells/well with different concentrations of endostatin fusion protein (1, 10,100 μg/ml). Cells will be allowed to migrate for 4 h at 37° C. At that time, the cells on the upper surface of the membrane are removed with a cell scraper, and the (migrated) cells on the lower surface are fixed in 3% formaldehyde and washed in PBS. Images of the fixed membrane is obtained using fluorescence microscopy at 550 nM with a digital camera, and the number of cells on each membrane is determined.

In Vitro Matrigel Assay:

Capillary tube formation assay in Matrigel is a useful in vitro assay to determine the branching morphogenesis of endothelial cells, which is a complex developmental program that regulates the formation of the blood vessels. Matrigel (Becton Dickinson, Franklin Lakes, N.J.) is used to coat a 24-well plate at 4° C. and allowed to polymerize at 37° C. for 30 min. HUVECs are seeded (5×10⁴ cells/well) on Matrigel-coated plates. Cells are incubated with VEGF (15 ng/ml) with or without endostatin fusion proteins or endostatin (1, 10, 100 μg/ml) in endothelial cell basal medium containing 2% FBS. After cells are incubated for 24 hrs or 96 hrs at 37° C., capillary tube formation is examined visually under a phase-contrast microscope and photographed. The intact tube number in six random views of ×100 magnification is counted.

Apoptotic Activities of Anti-HER2/neu Antibody-Endostatin Fusion Protein:

To analyze the mechanism of endostatin fusion protein action on endothelial cells and nonendothelial cells, C-PAE cells or HUVECs are tested for apoptosis by measuring annexin V-FITC staining with FACS and terminal deoxynucleotidyl transferase-mediated dUTP nick-end labeling assay (TUNEL staining). As addition of endostatin leads to a reduction in the antiapoptotic proteins Bcl-2 and Bcl-XL, expression of these antiapoptotic proteins in the presence of anti-HER2/neu antibody-endostatin fusion proteins are monitored by Western blot analysis.

Annexin V-FITC Staining Assay:

Annexin V, a calcium-dependent phospholipid-binding protein with a high affinity for phosphatidylserine (PS) is used to detect early stage apoptosis. C-PAE cells or HUVECs (2×10⁵) are plated onto a fibronectin-coated 6-well plate in DMEM containing 2% FCS and 3 ng/ml b-FGF. Different concentrations (1-100 μg/ml) of antibody-endostatin fusion proteins, control antibodies, or endostatin is added to each well, and cells will be harvested and processed 18 h after treatment. For the time course study, 10 μg/ml antibody-endostatin fusion proteins, control antibodies, or endostatin are added and cells are processed after 3, 4, 6, 12, and 18 h. Human recombinant TNF-α (40 ng/ml) is used as a positive control. The cells are washed in PBS and resuspended in binding buffer (10 mM HEPES/NaOH, pH 7.4, 140 mM NaCl, 2.5 mM CaCl₂). Annexin V-FITC is added to a final concentration of 100 ng/ml, and the cells are incubated in the dark for 10 min, then washed again in PBS and resuspended in 300 μl of binding buffer. 10 μl of propidium iodide (PI) is added to each sample before flow cytometric analysis. The cells are analyzed using a Becton Dickinson FACStar plus flow cytometer. In each sample, a minimum of 10,000 cells are counted and stored in listmode. Data analysis is performed with standard Cell Quest software (Becton-Dickinson).

Microscopic Detection of TUNEL Staining:

C-PAE cells or HUVECs are seeded at a density of 5,000 cells/well on fibronectin-coated (10 μg/ml) Lab-Tek chamber slides and grown in 0.4 ml of DMEM with 10% FCS. After 2 days, the old medium is aspirated and fresh DMEM with 2% FCS is added, and the cells are starved overnight. The following day, 0.36 ml of new medium (with 2% FCS) containing 3 ng/ml b-FGF are added along with antibody-endostatin fusion proteins, control antibodies, or endostatin (10 μg/ml) or TNF-α (20 ng/ml). For control samples, fresh medium (2% FCS) containing bFGF (3 ng/ml) is added. Following induction (24 h), the slides are washed twice with PBS, and subsequently fixed in fresh 4% formaldehyde/PBS at 4° C. for 25 min. The slides are washed in PBS and the cells permeabilized in 0.2% Triton X-100/PBS for 5 min on ice, then washed with fresh PBS twice for 5 min each at room temperature, and the TUNEL assay is performed as described in the ApoAlert DNA fragmentation assay kit (CLONTECH), except that the final concentration of propidium iodide (Sigma) used is 1 μg/ml. After the assay, a drop of anti-fade solution is added, and the treated portion of the slide is covered with a glass coverslip with the edges sealed with clear nail polish. Slides are viewed immediately under a fluorescent microscope using a dual filter set for green (520 nm) and red fluorescence (>620 nm). The images are captured using a digital camera. Images are imported into NHImage 1.59, and measurements of fluorescence intensity are obtained as positive pixels. For all samples except the positive control (TNF-α: 5 fields), 15 random fields are chosen, and the number of green and red cells per field are counted.

Western Blotting Analysis of Expression of Antiapoptotic Proteins, Bcl-2 or Bcl-XL:

C-PAE cells and HUVECs (1×10⁶) are seeded in 10-cm Petri dishes precoated with fibronectin (10 μg/ml) in the presence of 2% FCS containing 3 ng/ml b-FGF. Antibody-endostatin fusion proteins, control antibodies, or endostatin are added at 10 μg/ml, and cells are harvested at 12, 24, and 48 h after treatment. Cells are washed three times in PBS buffer, pH 7.4, and the cells are resuspended in 1 ml of 1×EBC buffer (50 mM Tris-HCl, pH 8.0, 120 mM NaCl, 1% Nonidet P-40) containing freshly added complete protease inhibitor tablet (Boehringer Mannheim), 100 μg/ml Pefabloc, 1 μg/ml pepstatin. The protein concentration in whole cell lysate is measured by the bicinchoninic acid (BCA) method (Pierce). 30 μg of whole cell extract is loaded onto a 4-15% gradient polyacrylamide gel. Transfer is performed using a semi-dry transblot apparatus (Bio-Rad). The membrane is blocked in wash buffer (1×Tris-buffered saline) with 5% nonfat dry milk and incubated at 37° C. for 1 h. Goat antibody against human Bcl-2 and mouse polyclonal antibody against Bax and Bcl-XL (Santa Cruz Biotechnology, Santa Cruz, Calif.) are used as primary antibodies. Polyclonal anti-actin antibody (Sigma) is used to normalize for protein loading. Secondary antibodies are anti-goat, mouse and rabbit immunoglobulin conjugated to HRP (Amersham Pharmacia Biotech). The immunoreactivity is detected with an enhanced chemiluminescence reagent (Pierce). Images are scanned using a flat bed scanner and quantitated by the NIH image 1.59 software. Normalization is calculated by dividing the Bcl-2 signal by that of actin within each experiment.

In Vivo Evaluation of the Antiangiogenic Properties of Anti-HER2/neu Antibody-Endostatin:

For in vivo antiangiogenesis, we test for effects of the fusion in Matrigel angiogenesis model in mice using VEGGF or VEGF with endostatin fusion proteins or endostatin alone. BALB/c mice (6-8 wk, n=3) are subcutaneously injected with 0.5 ml Matrigel (9-10 mg/ml) containing 150 ng/ml VEGF, with or without endostatin fusion proteins, antibody or endostatin (1 to 100 μg/ml), near the abdominal midline by using a 26-gauge needle. One week after Matrigel injection, mice are sacrificed, and the Matrigel plug, along with overlying skin and peritoneal membrane, is removed and fixed in 4% buffered formaldehyde in PBS. Plugs are embedded in paraffin, sectioned, and stained by incubation overnight at 4° C. with antibodies (DAKO Corporation, Carpenteria, Calif.) for endothelium-specific antigens such as PECAM (CD31) and proliferation cell nuclear antigen (PCNA) or Ki67 (MIB-1) to access endothelial cell proliferation. Thereafter, sections are treated with biotinylated antibody (ABC kit) for 40-45 min at room temperature, followed by a 45-min incubation with the avidin-biotin-peroxidase complex (ABC kit). The antigen-antibody complex is visualized by incubation with freshly prepared 3,3′-diaminobenzidine (DAB kit, Vector Laboratories). Sections are counterstained with hematoxylin-eosin. Ten fields are randomly selected using a microscope at 400× magnification, and photographed using a digital camera. The number of PECAM-1-positive, PCNA-positive, Ki67-positive cells are counted.

Antiangiogenic Activity of Anti-HER2/neu Antibody-Endostatins in Tumors by Immunohistochemical Staining:

BALB/c or BALB/c BCDM are injected s.c. in the flank region with 106 EMT6-HER2/neu or CT26-HER2/neu cells on the right flank and control 106 EMT6 or CT26 on the left flank, respectively. In addition, SCID mice are injected s.c. in the flank region with 106 MCF7-HER2/neu, SK-BR-3, or BT474 cells on the right flank and control 106 MCF7 on the left flank.

On the seventh day, mice are injected i.v. with the antibody-endostatin fusion proteins (42 μg), equimolar control antibodies (34 μg) or equimolar endostatin (8 μg). This treatment is repeated 7 times every other day. Visible tumors, along with overlying skin and surrounding tissue, are removed at various time points (2 days, 8 days, 16 days after treatments, 3 mice/time point), and tissue sections are immunohistochemically stained with mouse antibody specific for endothelium-specific antigens such as PECAM (CD31, DAKO Corporation, Carpenteria, Calif.), and doubly stain tissue with mouse antibody specific for proliferation cell nuclear antigen (PCNA, DAKO Corporation, Carpenteria, Calif.) or Ki67 (MIB-1, DAKO Corporation, Carpenteria, Calif.) to access endothelial cell proliferation.

Tissue sections are fixed in 4% paraformaldehyde/PBS pH 7.4, dipped in a quenching solution (3% hydrogen peroxide/60% methanal) to remove endoperoxidase activity, and then placed in 10% normal blocking serum (ABC kit, Vector Laboratories, Inc., Burlingame, Calif.) for 20 min before incubation overnight at 4° C. with mouse antibody for PECAM-1, Ki67, or PCNA, or with mouse IgG as the control. Thereafter, tissue sections are treated with biotinylated antibody (ABC kit) for 40-45 min at room temperature, followed by a 45-min incubation with the avidin-biotin-peroxidase complex (ABC kit). The antigen-antibody complex is visualized by incubation with freshly prepared 3,3′-diaminobenzidine (DAB kit, Vector Laboratories), and the tissue is counterstained with hematoxylin. Ten fields are randomly selected using a microscope at 400× magnification, and photographed using a digital camera. The number of PECAM-1-positive, PCNA-positive, Ki67-positive cells are counted.

Antiangiogenic Activity of Anti-HER2/neu Antibody-Endostatin on VEGF Expression and Neovascularization in Tumors:

Anti-tumor activity of endostatin is associated with a down-regulation of VEGF expression. The experiment shown above is repeated in human breast cancer xenografts (SK-BR-3, BT474, MCF7 and MCF7-HER2) in SCID mice (n=3) to evaluate antiangiogenic activity of the antibody-endostatin fusion proteins (42 μg), equimolar control antibodies (34 μg) or equimolar endostatin (8 μg). This treatment is repeated 7 times every other day. Visible tumors, along with overlying skin and surrounding tissue, are removed at various time points (2 days, 8 days, 16 days after treatments, 3 mice/time point). Tissues are stained for endothelial cell proliferation using PCNA or Ki67 as described above, and the tissue sections are also stained with specific antibodies (VEGF-A, Neomarker; VEGF-C and VEGF-D, Santa Cruz Biotechnology, Santa Cruz, Calif.) for VEGF family.

Serum is collected at various times (before treatment, 2 days, 8 days, 16 days after treatments) for VEGF ELISA (R&D Systems, MN) to measure antiangiogenic abilities of endostatin fusion proteins as described above. In disease states, VEGF can be detected in various tumor cells and five different VEGF isoforms, with 121, 145, 165, 189, and 206 amino acids, can be generated as a result of alternative splicing from the single VEGF gene. These isoforms differ in their molecular mass and in their biological properties, such as their ability to bind to heparin or heparan-sulphate proteoglycans and to different VEGF receptors (VEGFRs). The splice forms VEGF₁₂₁, VEGF₁₄₅, and VEGF₁₆₅ are secreted, whereas VEGF₁₈₉ is tightly bound to cell surface heparan-sulphate and VEGF₂₀₆ is an integral membrane protein. In contrast to the other forms, VEGF₁₂₁, does not bind to heparin or extracellular matrix proteoglycans. The signalling tyrosine kinase receptors VEGFR-1 (flt-1, fms-like tyrosine kinase 1) bind VEGF₁₂₁ and VEGF₁₆₅, and VEGFR-2 (KDR, kinase domain region/flk-1, fetal liver kinase 1) additionally VEGF₁₄₅ (apart from certain VEGF-related peptides). mRNA expression of VEGF isoforms is determined in excised tumors by RT-PCR. For RT-PCR, frozen samples (1 g) are crushed in an achate mortar under liquid nitrogen; RNA is isolated by the phenol-guanidinium thiocyanate method and purified by isopropanol and repeated ethanol precipitation; and contaminating DNA is destroyed by digestion with RNase-free DNase 1 (20 min at 25° C.). After inactivation of the DNase (15 min at 65° C.), cDNA is generated with 1 μl (20 pmol) of oligo (dT)15 primer (Amersham) and 0.8 μl of superscript RNase H-reverse transcriptase (Gibco) for 60 min at 37° C. For PCR, 4 μl of cDNA is incubated with 30.5 μl of water, 4 μl of 25 mM MgCl₂, I PI of dNTP, 5 μl of 10×PCR buffer, 0.5 μl (2.5 U) of platinum Taq DNA polymerase (Gibco), and the following primers (2.5 μl each containing 10 pmol): non-selective for all VEGF splice variants 5′-ATG-GCA-GAA-GGG-CAG-CAT-3′ (sense) and 5′-TTG-GTG-AGG-TTT-GAT-CCG-CAT-CAT3′ (antisense) yielding a 255 bp fragment (40 cycles, annealing temperature 55° C.); selective for VEGF splice variants 5′-CCA-TGA-ACT-TTC-TGC-TGT-CTT-3′ (sense) and 5′-TCG-ATC-GTT-CTG-TAT-CAG-TCT-3′ (antisense) yielding a different fragment size for each variant (40 cycles, annealing temperature 55° C.). With selective primers, the 526 bp product corresponds to VEGF₁₂₁, the 598 bp product to VEGF₁₄₅, the 658 bp product to VEGF₁₆₅, the 730 bp product corresponds to VEGF₁₈₉, and the 781 bp product corresponds to VEGF₂₀₆.

### Example 1

### Chimeric Molecules

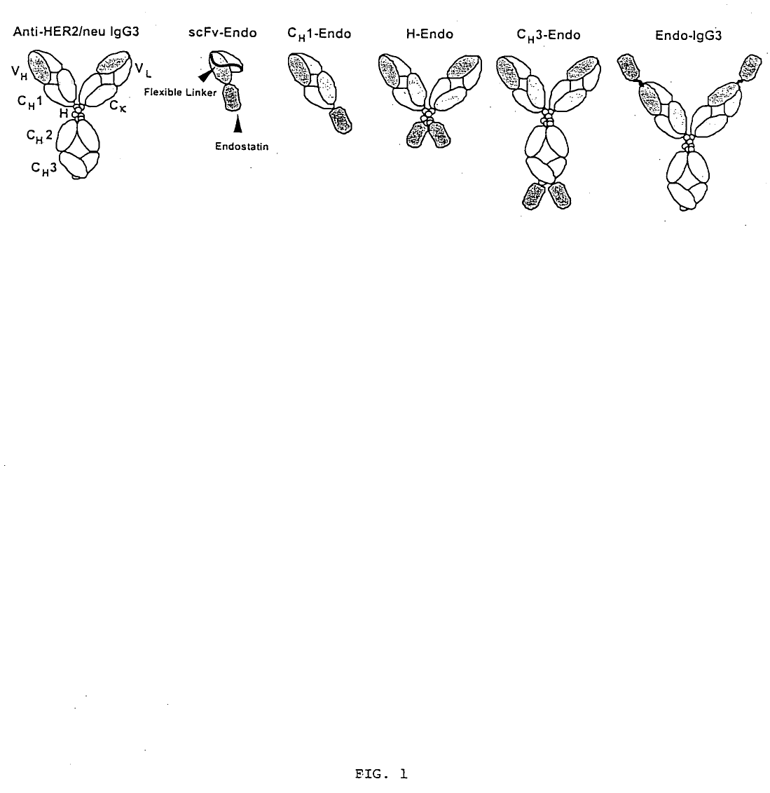

Referring to FIG. 1 several Ig-endostatin chimeric molecules are illustrated. These include anti-HER2/neu scFv-Endo, anti-HER2/neu IgG3-C_{H}1-Endo, anti-HER2/neu IgG3-H-Endo, and Endo-anti-HER2/neu IgG3 fusion proteins. In one method to produce antibody fusion proteins, vectors that contain unique restriction sites at the 3′ end of the C_{H}1 exon, immediately after the hinge, or at the 3′ end of the C_{H}3 exon as well as on the variable domains of both human kappa light chain and IgG3 heavy chain IgG3 are used. Using these constructs, as illustrated in FIG. 1, endostatin can be joined to anti-HER2/neu after C_{H}1 of anti-HER2/neu IgG3; and endostatin of Endo-IgG3 can be joined at the amino-terminus of the variable region with flexible linker (Gly₄-Ser)₃. The Fv genes of anti-HER2/neu heavy (FvH) and light chain (FvL) variable region genes can be cloned by PCR, and the cloned Fv gene fragments joined with a flexible linker (Gly₄-Ser)₃. Endostatin is joined at the 3′ end of the FvL-(Gly₄-Ser)₃-FvH gene to form scFv-Endo. The constructed fusion genes can be expressed in myeloma cell line SP2/0. For example, transfectomas expressing anti-HER2/neu IgG3-C_{H}3-Endo fusion, endostatin, anti-HER2/neu IgG3, and anti-dansyl IgG3 of unrelated control specificity have been generated. To purify the fusion proteins produced by the host cells, the proteins are isolated from culture medium through protein A affinity chromatogaraphy for C_{H}3-Endo and Endo-IgG3, or using heparin affinity chromatography (which binds to the endostatin moiety) for scFv-Endo, C_{H}1-Endo, and H-Endo which lack a protein A-binding site. For fusion proteins containing both heavy and light chains, size and assembly into H₂L₂ form is assessed using SDS-PAGE. Western blotting analysis with rabbit anti-endostatin sera can be used to detect the attached endostatin moiety. Expected characteristics of the fusion proteins are shown below.

| | “Predicted” Properties of Fusion Proteins | | | |
|---|---|---|---|---|
| | | HER2/neu | | |
| | Tumor | Binding | Serum | Effector |
| Recombinant Proteins | Penetration | Ability | Half-Life | Function |
| IgG3 Heavy Chain | ++ | ++++ | +++ | Yes |
| Single Chain Fv-Endo | ++++ | ++ | + | No |
| (scFv-Endo) | | | | |
| IgG3-C_{H}1-Endo | +++ | +++ | ++ | No |
| (C_{H}1-Endo) | | | | |
| IgG3H-Endo | ++ | ++++ | +++ | No |
| (H-Endo) | | | | |
| IgG3-C_{H}3-Endo | ++ | ++++ | +++ | Yes |
| (C_{H}3-Endo) | | | | |
| Endo-IgG3 | ++ | ++++ | +++ | Yes |

### Example 2

### Serum Stability Studies

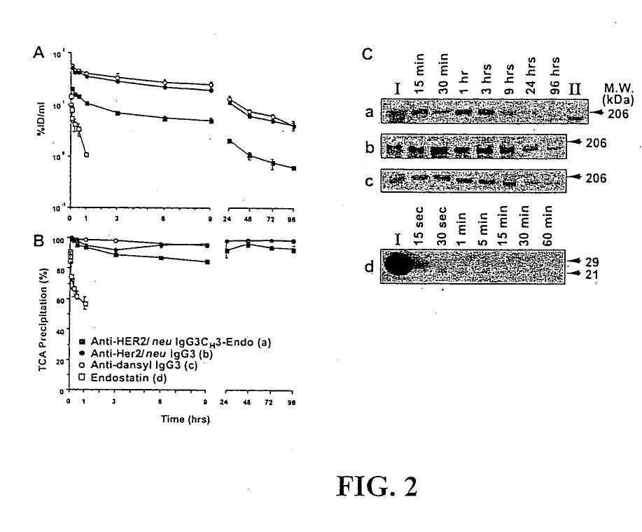

To characterize the in vivo pharmacokinetic patterns of the antibody-endostatin fusion protein, mice with/without implanted tumors (CT26 or CT26-HER2/neu) were injected intravenously with [¹²⁵I] labeled anti-HER2/neu IgG3, anti-HER2/neu IgG3-C_{H}3-Endo, endostatin, and a control anti-dansyl IgG3 and clearance of endostatin on fusion measured. Referring to FIG. 2A, [¹²⁵I]-endostatin was rapidly removed from the plasma compartment in mice with/without tumors (T_{1/2}² elimination: 0.5-3.8 hrs), while the rate of removal of [¹²⁵I] labeled anti-HER2/neu IgG3-C_{H}3-Endo (T_{1/2} ²: 40.2-44.0 hrs) was similar to those of [¹²⁵I] labeled anti-HER2/neu IgG3 (T_{1/2}²: 39.9-63.8 hrs) and control anti-dansyl IgG3 (T_{1/2}²: 43.7-46.5 hrs).

To analyze the serum stability of [¹²⁵I] labeled anti-HER2/neu IgG3, anti-HER2/neu IgG3-C_{H}3-Endo, endostatin and anti-dansyl IgG3 plasma samples were TCA-precipitated and counted (FIG. 2B). 96 hours following injection approximately 90% of the anti-HER2/neu IgG3 and anti-HER2/neu IgG3-C_{H}3-Endo in serum remained intact. Endostatin was rapidly eliminated with little remaining in the circulation by 60 min. For endostatin, approximately 90% was intact 2 min after injection and only 55% of the remaining circulating endostatin remained intact at 60 min. In contrast anti-HER2/neu IgG3-C_{H}3-Endo cleared much more slowly with kinetics resembling anti-HER2/neu IgG3 and anti-dansyl IgG3. Analysis of serum samples by SDS-PAGE confirmed that the anti-HER2/neu IgG3-C_{H}3-Endo in circulation remained intact (FIG. 2C).

### Example 3

### Biolocalization Studies

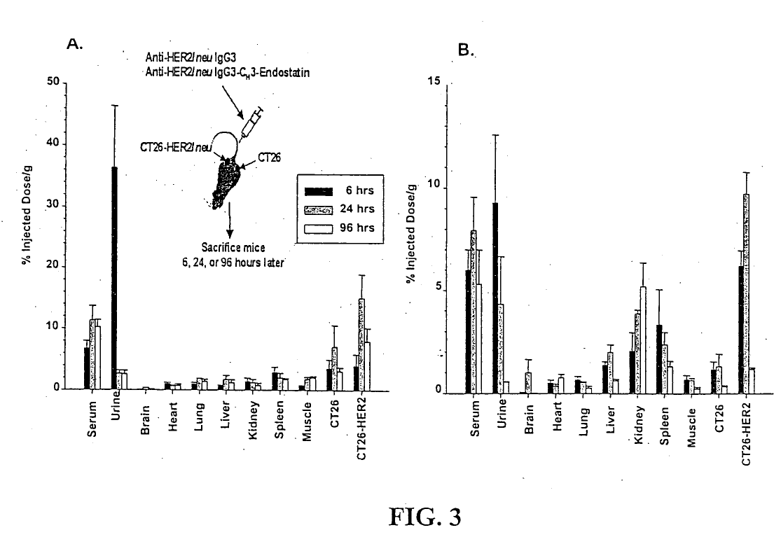

To measure biodistribution and biolocalization of the endostatin fusion protein, purified endostatin fusion protein was labeled with ¹²⁵I. Referring to FIG. 3 and the table immediately below, 96 hours following an intravenous injection into mice bearing tumors, the radiolocalization indices (the % injected dose[ID]/g in tumor divided by the % ID/g in blood) of anti-HER2/neu IgG3-C_{H}3-Endo and anti-HER2/neu IgG3 were similar. Anti-HER2/neu IgG3-C_{H}3-Endo showed a tumor/blood ratio of 3.76 for CT26-HER2/neu and a 0.50 tumor/blood ratio for CT26; whereas anti-HER2/neu IgG3 showed 2.83 and 0.47 ratios for CT26-HER2/neu and CT26, respectively. No enhanced targeting to tumors was seen for endostatin alone. Therefore, both anti-HER2/neu antibody and anti-HER2/neu antibody-endostatin fusion protein retain the ability to localize to HER2/neu bearing tumors.

In mice simultaneously implanted with CT26 and CT26 expressing HER2/neu (CT26-HER2) tumors on opposite flanks, ¹²⁵I-labeled anti-HER2/neu IgG3-endostatin fusion protein and anti-HER2/neu IgG3 preferentially localized to CT26-HER2 tumors. Specific tumor radiolocalization indices of anti-HER2/neu IgG3-endostatin were actually greater than those of anti-HER2/neu IgG3 in several separate experiments. This indicated relative localization of targeted antibody-endostatin fusions to tumor due to binding to HER2/neu target antigen.

| | Time | | CT26-HER2 | Radiolocalization |
|---|---|---|---|---|
| Treatment | (Hrs) | CT26 (% ID/g) | (% ID/g) | Indices* |
| Anti-HER2/ | 6 | 3.51 ± 1.38 | 3.94 ± 1.83 | 1.12 |
| neu IgG3 | 24 | 7.04 ± 3.48 | 14.95 ± 3.48 | 2.12 |
| | 96 | 3.03 ± 0.63 | 7.88 ± 2.18 | 2.60 |
| Anti-HER2/ | 6 | 1.16 ± 0.38 | 6.20 ± 0.76 | 5.34 |
| neu IgG3- | 24 | 1.31 ± 0.60 | 9.72 ± 1.05 | 7.42 |
| endostatin | 96 | 0.33 ± 0.05 | 1.17 ± 0.07 | 3.55 |

*Radiolocalization Indices represent the ratios of the % ID/g in CT26-HER2 divided by the % ID/g in CT26.

### Example 4

### Anti-Tumor Studies

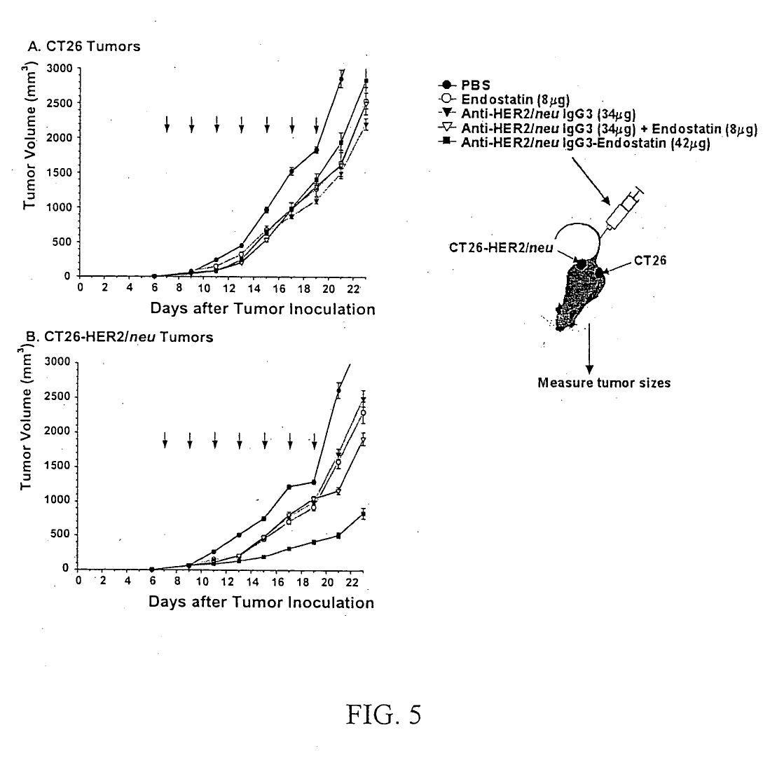

The ability of anti-HER2/neu IgG3-C_{H}3-Endo, anti-HER2/neu IgG3 and endostatin to preventing the growth of CT26 expressing HER2/neu in BALB/c mice was examined. The results of these experiments are shown in FIG. 5. BALB/c mice were subcutaneously injected with 1×10⁶ cells and tumor growth measured. On day 7, most of mice developed palpable tumors (about 5 mm in diameter) and the treatment of mice bearing tumors (n=5 per group) initiated every other day by intravenous injection (5 times) of anti-HER2/neu IgG3-C_{H}3-Endo, anti-HER2/neu IgG3, anti-dansyl IgG3, endostatin, or PBS controls. Referring to FIG. 5A, tumor growth in mice treated with anti-HER2/neu IgG3 or endostatin was reduced relative to anti-dansyl IgG3 or PBS controls. Treatment with anti-HER2/neu IgG3-C_{H}3-Endo resulted in additional reduction in tumor volume. Anti-HER2/neu IgG3-C_{H}3-Endo demonstrated significant growth inhibition (p<0.05) compared with PBS, anti-HER2/neu IgG3 or endostatin administered at two-fold molar excess relative to anti-HER2/neu IgG3-endostatin alone. A ten-fold increase in endostatin alone further increased efficacy.

Referring to FIG. 5B, in mice simultaneously implanted with CT26, and CT26-HER2/neu on opposite flanks, equimolar administration of anti-HER2/neu IgG3-endostatin to mice showed preferential inhibition of CT26-HER2/neu, compared to contralaterally implanted CT26 parental tumor. Anti-HER2/neu IgG3-endostatin inhibited more effectively than endostatin, anti-HER2/neu IgG3 antibody, or the combination of antibody and endostatin (p<0.05).

Whether anti-HER2/neu IgG3-endostatin, endostatin, anti-HER2/neu IgG3 antibody, or the combination of antibody and endostatin would inhibit the growth of the human breast cancer SK-BR-3 in SCID mice was examined. SCID mice (n=8 per group) were subcutaneously injected with 1×10⁶ cells of SK-BR-3 and tumor growth measured. By day 15, most mice developed palpable tumors (about 5 mm in diameter) and treatment was initiated every other day with intravenous injection (10 times) of anti-HER2/neu IgG3-C_{H}3-Endo, anti-HER2/neu IgG3, endostatin, or the combination of antibody and endostatin. Mice treated with anti-HER2/neu IgG3-C_{H}3-Endo, endostatin, anti-HER2/neu IgG3 antibody, or the combination of antibody and endostatin all showed inhibition of tumor growth. Administration of anti-HER2/neu IgG3-C_{H}3-Endo consistently resulted in the greater reduction of tumor volume, compared to either anti-HER2/neu antibody alone, endostatin alone, or antibody and endostatin given in combination (p<0.05).

### Example 5

### Production and Characterization of Anti-HER2/neu IgG3-Endostatin

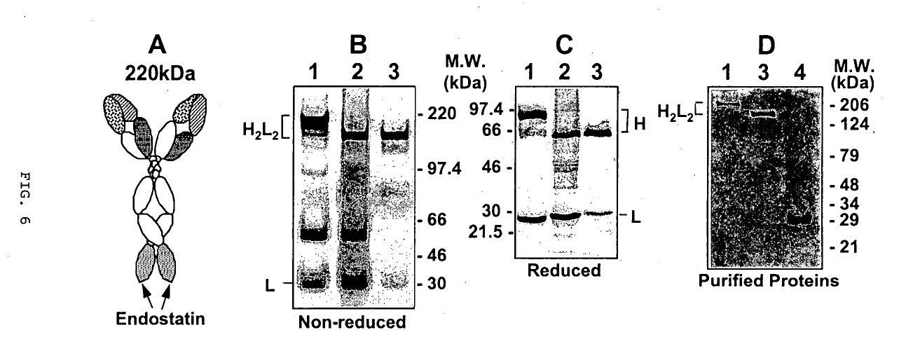

The anti-HER2/neu antibody-endostatin fusion protein of the expected molecular weight was produced and secreted from the stably transfected Sp2/0 cell lines as the fully assembled H₂L₂ form (FIG. 6). The secreted ³⁵S-methionine labeled anti-HER2/neu IgG3-endostatin has a molecular weight of approximately 220 kDa under non-reducing conditions (FIG. 6B), the size expected for a complete antibody (170 kDa) with 2 molecules of endostatin (25 kDa) attached. Following reduction (FIG. 6C), H and L chains of the expected molecular weight were observed. To confirm that the endostatin moiety was present in the anti-HER2/neu IgG3-endostatin protein, purified anti-HER2/neu IgG3-endostatin and endostatin were resolved under non-reducing conditions (FIG. 6D). Following Western blotting, anti-HER2/neu IgG3-endostatin was identified at the molecular weight of 220 kDa by both anti-human IgG or anti-endostatin antibody. Following reduction, the predominant heavy chain band from anti-HER2/neu IgG3-endostatin migrated at the expected size of 85 kDa.

### Example 6

### Antiangiogenic Activity of Anti-HER2/neu IgG3-Endostatin

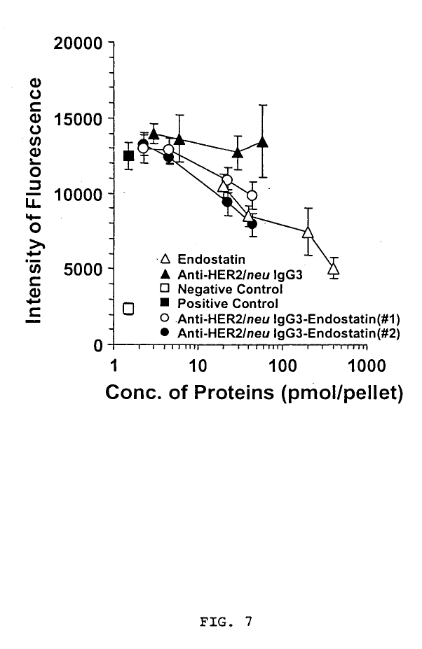

The ability of endostatin to block VEGF/bFGF-induced angiogenesis in vitro was tested using the chorioallantoic membrane (CAM) assay. Pellets containing Vitrogen and VEGF/bFGF (100 ng and 50 ng/pellet, respectively) and either anti-HER2/neu IgG3 (0.5-10 μg/pellet: 2.95-59 pmol/pellet), anti-HER2/neu IgG3-endostatin (0.5-10 μg/pellet: 2.25-45 pmol/pellet), or endostatin (0.5-10 μg/pellet: 20-400 pmol/pellet) were measured for invasion of new capillaries (FIG. 7). Two independent preparations of anti-HER2/neu antibody-endostatin fusion protein were able to suppress the angiogenic response mediated by VEGF/bFGF in a dose-dependent manner with a specific activity similar to that seen with endostatin (FIG. 7). In contrast anti-HER2/neu IgG3 showed no anti-angiogenic response (FIG. 7). Therefore genetically engineered anti-HER2/neu-IgG3-endostatin maintains the ability to inhibit the angiogenic response mediated by VEGF/bFGF.

### Example 7

### Serum Clearance and Stability of Anti-HER2/neu IgG3-Endostatin

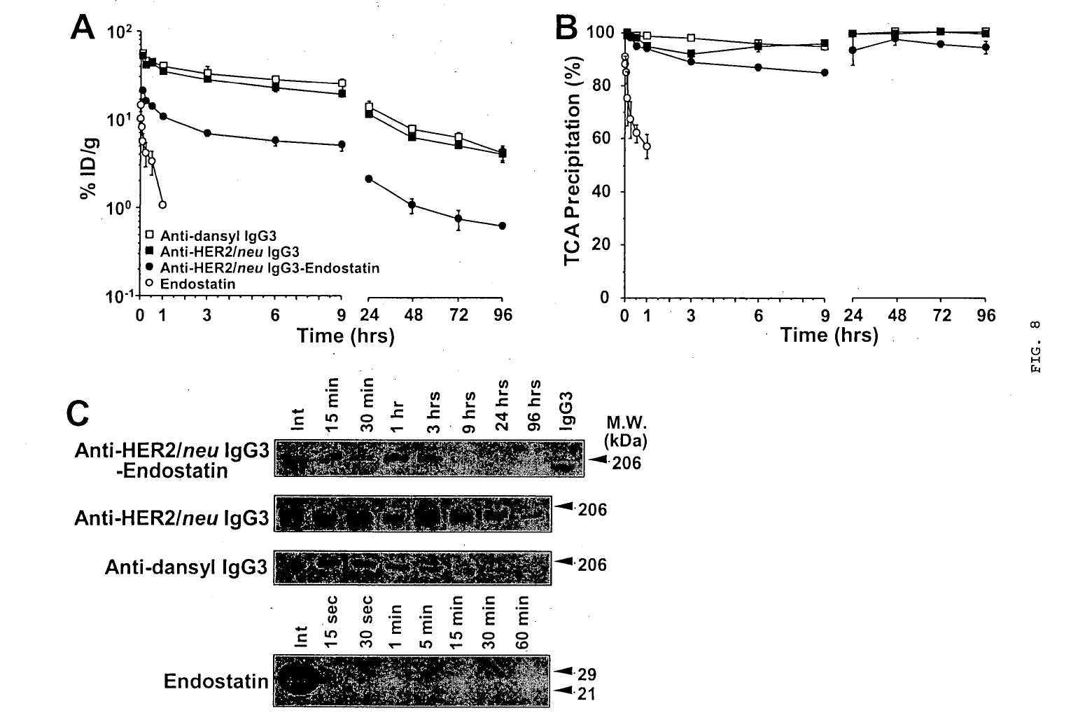

To characterize the pharmacokinetics of anti-HER2/neu IgG3-endostatin, mice with/without implanted tumors (CT26 or CT26-HER2) were injected intravenously with [1251]-anti-HER2/neu IgG3, anti-HER2/neu IgG3-endostatin, endostatin, or a control anti-dansyl IgG3 and clearance of injected radiolabeled proteins measured. Representative results from mice with implanted HER2/neu-expressing CT26 tumors are shown in FIG. 8 and the pharmacokinetic data for mice in all groups are summarized in Table 1. [¹²⁵I]-endostatin was rapidly removed from the plasma compartment in mice with or without tumors (T_{1/2}² elimination: 0.5-3.8 hrs), while the clearance rate of [¹²⁵I]-anti-HER2/neu IgG3-endostatin (T_{1/2}²: 40.2-44.0 hrs) was similar to that of [¹²⁵I]-anti-HER2/neu IgG3 (T_{1/2}²: 39.9-63.8 hrs) and anti-dansyl IgG3 (T_{1/2}²: 43.7-46.5 hrs) (FIG. 8A and Table 1). Therefore endostatin fused with antibody is cleared from the peripheral compartment much more slowly than endostatin alone.

In mice bearing CT26-HER2 tumors (Table 1), the area under the plasma concentration curve (AUC) of anti-HER2/neu IgG3-endostatin was increased by a factor of 56 (13,100% IDmin/ml vs. 233% IDmin/ml) compared to endostatin, as a consequence of both a longer half-life of elimination (69 fold increase: 2,640 min vs. 38 min) and an increased “mean residence time” (MRT) (56 fold increase: 2800 min vs. 50 min). Endostatin was very rapidly removed from serum within 30 min, principally by glomerular filtration and renal clearance, but anti-HER2/neu IgG3-endostatin demonstrated much slower clearance from serum, similar to those of anti-HER2/neu IgG3 and anti-dansyl IgG3.

To analyze the serum stability of [¹²⁵I] labeled anti-HER/neu IgG3, anti-HER2/neu IgG3-C_{H}3-endostatin, endostatin and anti-dansyl IgG3, plasma samples were TCA-precipitated and counted (FIG. 8B). 96 hours following injection approximately 90% of the anti-HER2/neu IgG3 and anti-HER2/neu IgG3-endostatin in serum remained intact. For endostatin, approximately 90% was intact 2 min after injection and only 55% of the remaining circulating endostatin remained intact at 60 min. In contrast anti-HER2/neu IgG3-endostatin cleared much more slowly with kinetics resembling anti-HER2/neu IgG3 and anti-dansyl IgG3. Analysis of serum samples by SDS-PAGE confirmed that the anti-HER/neu IgG3-endostatin in circulation remained intact (FIG. 8C). Thus, the antibody moiety of anti-HER2/neu IgG3-endostatin fusion protein renders the genetically fused endostatin much more stable in the blood stream.

### Example 8

### Biodistribution and Biolocalization of Anti-HER2/neu IgG3-Endostatin

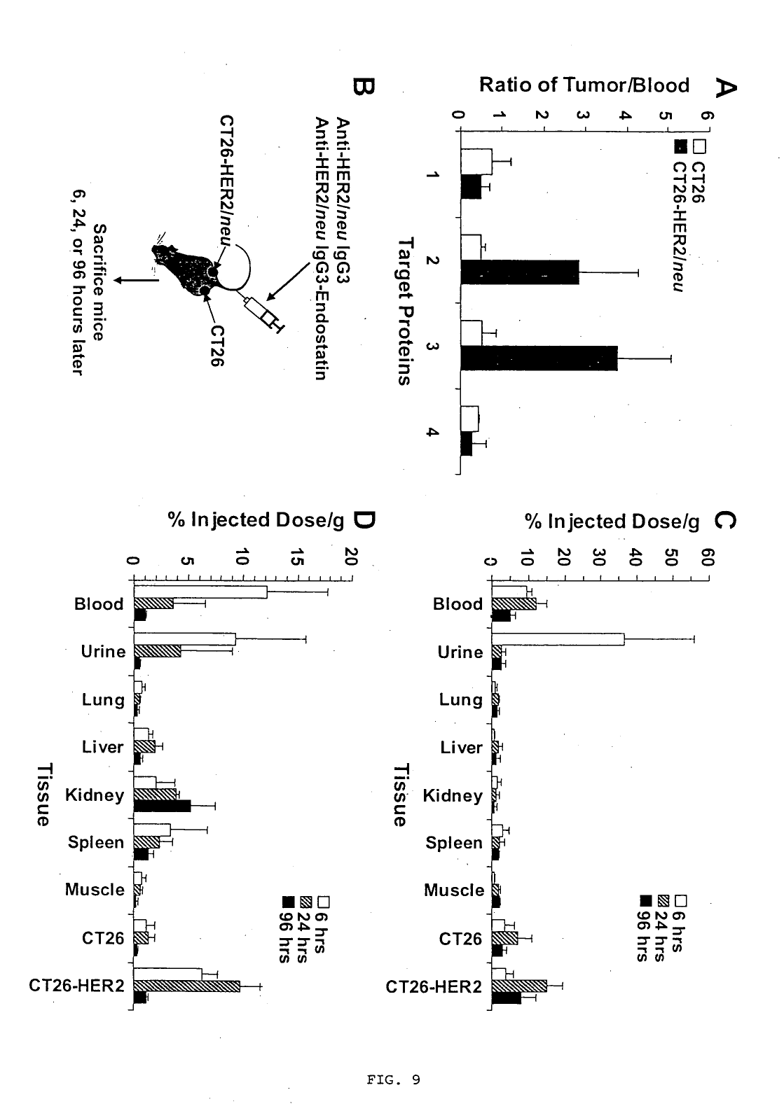

Ninety-six hours following an intravenous injection of mice bearing CT26-HER2 tumors, anti-HER2/neu IgG3 was found mainly in the tumor and blood (5.67 and 2.10% ID/g, respectively). The radiolocalization indices at 96 hours post injection (the % ID/g in tumor divided by the % ID/g in blood) of anti-HER2/neu IgG3-endostatin and anti-HER2/neu IgG3 were similar (FIG. 9A). Anti-HER2/neu IgG3-endostatin showed a tumor/blood ratio of 3.76 for CT26-HER2 and a 0.50 for CT26, whereas anti-HER2/neu IgG3 showed tumor/blood ratios of 2.83 and 0.47 for CT26-HER2 and CT26, respectively (FIG. 9A). Therefore, both anti-HER2/neu antibody and anti-HER2/neu antibody-endostatin fusion protein preferentially localized to HER2/neu expressing tumors.

To measure localization of antibody-endostatin fusion proteins to the antigenic target, mice simultaneously implanted with CT26 and CT26-HER2 tumors on opposite flanks were injected intravenously with either ¹²⁵I-labeled anti-HER2/neu IgG3-endostatin fusion protein or ¹²⁵I-labeled anti-HER2/neu antibody (FIG. 9, Table 2). The biodistribution and biolocalization of the labeled proteins was examined at different times (6, 24, and 96 hours) after injection of labeled proteins (FIG. 9B). Anti-HER2/neu IgG3-endostatin fusion protein and anti-HER2/neu IgG3 preferentially localized to CT26-HER2 tumors (FIGS. 9C and 9D). Specific tumor radiolocalization indices of anti-HER2/neu IgG3-endostatin were actually greater than those of anti-HER2/neu IgG3 (FIG. 9, Table 2). This indicates that the relative localization of targeted antibody-endostatin fusions to tumor is due to binding to the HER2/neu target antigen (Table 2).

### Example 9

### Anti-Tumor Activities of Anti-HER2/neu IgG3-Endostatin In Vivo

Murine colon adenocarcinoma CT26 cells were transduced with the gene for HER2/neu antigen as previously described. The CT26-HER2 cells have been used in these studies and proliferated at the same rate in vitro as parental CT26 cells. Preliminary experiments revealed that the CT26-HER2 tumors implanted in BALB/c mice grew at the same rate as the parental CT26 tumors (Ref. Lab Animal).

In preliminarily experiments, we studied the anti-tumor effects of anti-HER2/neu IgG3-endostatin, anti-HER2/neu IgG3 and endostatin on the growth of CT26-HER2 in BALB/c mice. Tumor growth in mice treated with anti-HER2/neu IgG3 or endostatin was reduced relative to an isotype control anti-dansyl IgG3 or PBS control. Treatment with anti-HER2/neu IgG3-endostatin resulted in additional reduction in tumor volume. Anti-HER2/neu IgG3-endostatin demonstrated significantly better growth inhibition when compared to PBS, anti-HER2/neu IgG3 or endostatin administered. Genetic fusion of endostatin to the anti-HER2/neu IgG3 initially appeared to inhibit tumor growth more efficiently than either anti-HER2/neu IgG3 or endostatin alone.

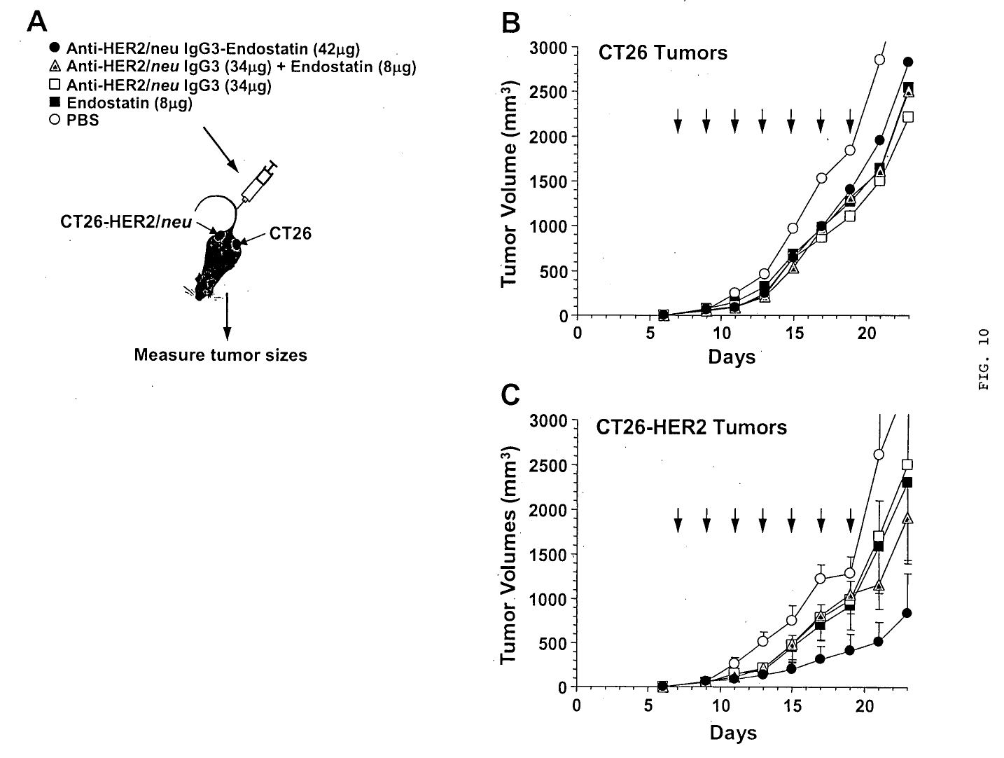

To confirm the preliminary experiments, mice were simultaneously implanted with CT26, and CT26-HER2 on opposite flanks. Administration of anti-HER2/neu IgG3-endostatin showed preferential inhibition of CT26-HER2 growth, compared to contralaterally implanted CT26 parental tumor (FIG. 10). Anti-HER2/neu IgG3-endostatin inhibited more effectively than endostatin, anti-HER2/neu IgG3 antibody, or the combination of antibody and endostatin p<0.05).

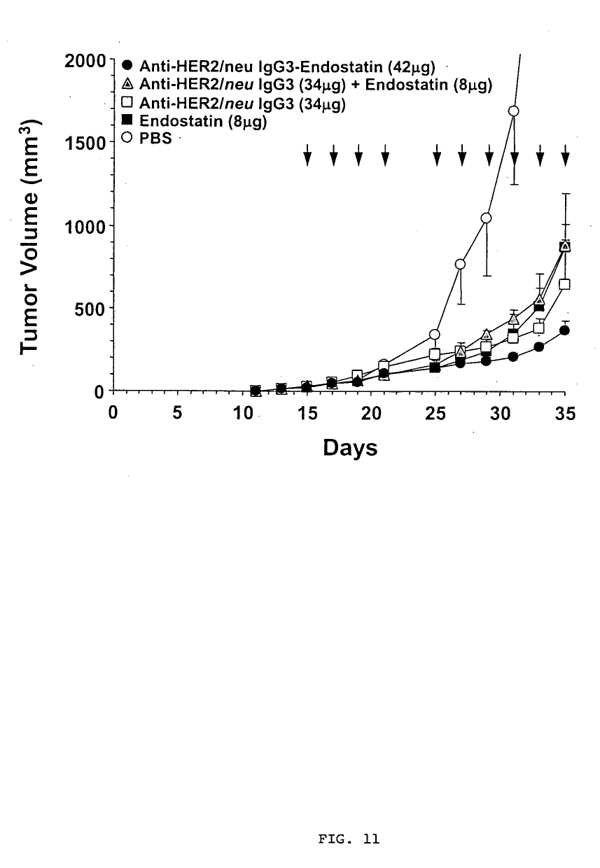

Herceptin, anti-HER2/neu IgG1, was able to inhibit the growth of SK-BR-3 breast carcinoma cells, which overexpress HER2/neu. It was next determined whether anti-HER2/neu IgG3-endostatin, endostatin, anti-HER2/neu IgG3 antibody, or both antibody and endostatin in combination would inhibit the growth of human breast cancer SK-BR-3 xenografts in SCID mice. SK-BR-3 was implanted on the flank of SCID mice. The treatment was repeated 10 times (FIG. 11). Administration of anti-HER2/neu IgG3-endostatin resulted in a greater reduction of tumor volume, compared to either anti-HER2/neu antibody alone, endostatin alone, or antibody and endostatin given in combination (p<0.05) (FIG. 11).

### Example 10

### Blood Vessel Formation in CT26-HER2 Tumors Treated with the Anti-HER2/neu IgG3-Endostatin Fusion Protein

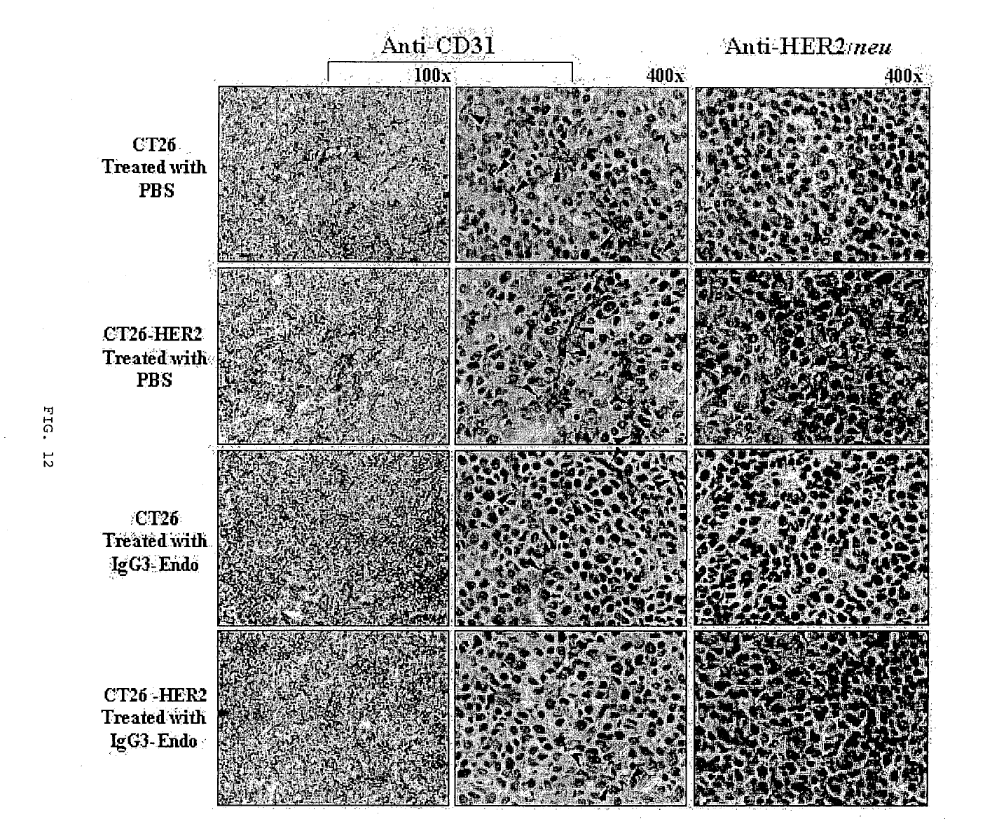

To better understand the mechanism of anti-tumor activity of the anti-HER2/neu IgG3-endostatin fusion protein, blood vessel formation in tumors was analyzed. Mice were simultaneously implanted with CT26 and CT26-HER2 tumors on opposite flanks and allowed to grow until the tumor diameter was 4-6 mm at which time the mice were intravenously treated with either anti-HER2/neu IgG3-endostatin fusion proteins or PBS. CT26-HER2 tumors grew slower in mice treated with anti-HER2/neu IgG3-endostatin compared to the others with kinetics similar to those in FIG. 10. After the fifth treatments, the tumors were removed and cryosections of tumors were immunohistochemically stained for endothelial cells with anti-PECAM-1 antibody to visualize the blood vessel formation of these tumors (FIG. 12). The parental CT26 tumor tissue and the untreated CT26-HER2 tumor tissue appeared to have more vessels than CT26-HER2 treated with endostatin fusion proteins.

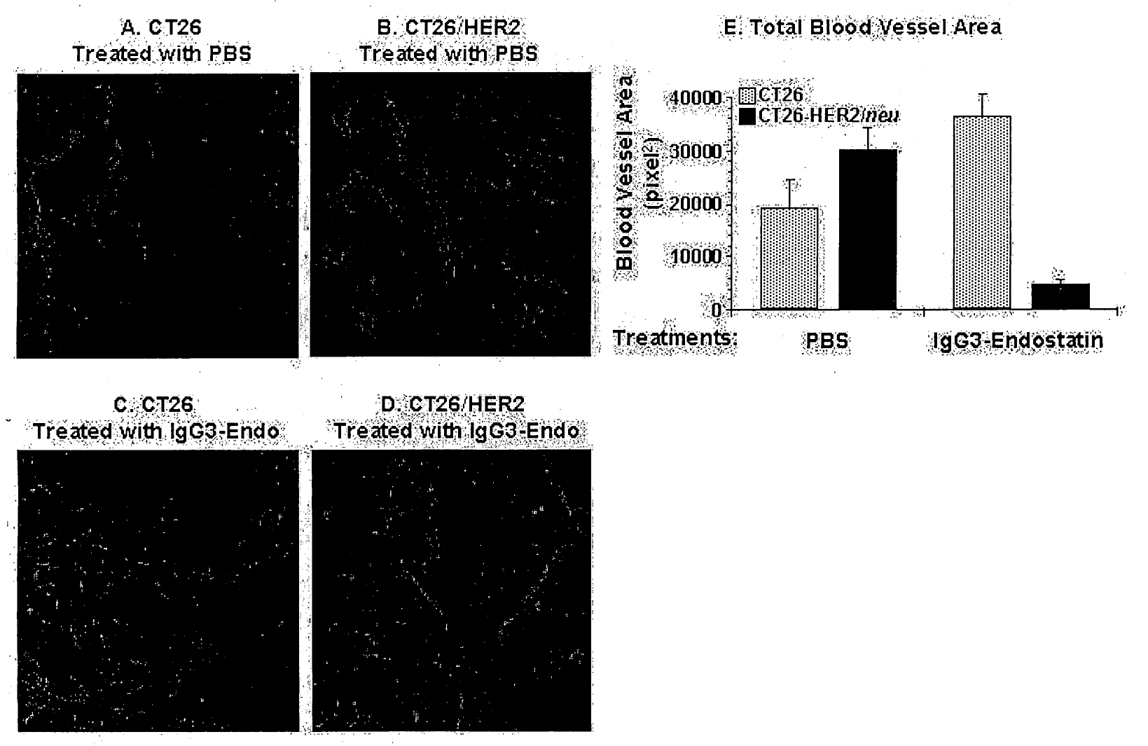

To distinguish the blood vessel formation, the tumor sections were stained with rat anti-mouse anti-PECAM antibody, detected with anti-rat IgG-Alexa 594, and then analyzed through confocal microscope (FIG. 13). Confocal microscopic analysis for these tumors revealed striking differences in the vasculature between CT26-HER2 tumors treated with anti-HER2/neu IgG3-endostatin and the others, which may explain the altered tumor growth observed in FIG. 10. Images composed of 14-21 digital microscopic images showed that blood vessels in the parental CT26 tumors with/without endostatin fusion treatments and in PBS-treated CT26-HER2 tumors appear more organized and branched than are the blood vessels in the CT26-HER2 tumors treated with anti-HER2/neu IgG3-endostatin (FIG. 13A-D).

The alterations in vasculature were quantified by measuring the blood vessel density. The blood vessel density was measured by determining the area that was occupied by vessels, which provides the amount of vascular area within each tumor. Using this measure, the HER2/neu expressing tumors with endostatin fusion treatments had significantly less vascular area (16%) than did the untreated CT26-HER2 tumors (FIG. 13E, Table 3).

### Example 11

### Angiogenic Effects of VEGF Ischemic/Non-Ischemic Tissues

Antiangiogenic effects of the antibody-endostatin fusion proteins will be investigated using animal hindlimb models of therapeutic angiogenesis. Rat or rabbit hindlimb ischemia models are available. The ischemic levels in the rabbit model can be manipulated as maximal, severe, or moderate ischemic conditions.

Rabbit Hindlimb Ischemia Model:

The normal distribution of arteries and capillaries 1 h after surgery (iliac tie and femoral excision), flow through the iliac and femoral arteries was eliminated indicating ischemia. Although there has been significant collateral development and return of flow to the limb, flow through the femoral artery and its associated vessels is still absent. We did not detect significant inflammation, necrosis, or tissue loss despite the severe early ischemia indicating that the muscle is significantly reperfused. The arrow indicates the position of the excised femoral artery. In contrast, the VEGF-treated limb recovered full flow to the distal branches of the femoral artery. Quantitation of these vessels from the original angiography revealed >10-fold more collateral vessels with external diameter >100 μm in the treated limbs. The generation of new vessels in the VEGF treated limbs could involve combinations of vasculogenesis, angiogenesis, and arteriogenesis.

Angiogenic Effects of VEGF in Non-Ischemia Model:

Neovascularization of non-ischemic tissues has been examined at the rat subcutaneous peritoneal fat pad and mouse ear flap. In both case sutures were tied into the tissues and 2×10⁹ pfu of Ad-CMV-VEGF or Ad-β-Gal were injected around the sutures. Tissues were analyzed after 3-weeks. New vessels were clearly visible in both tissues injected with Ad-CMV-VEGF but not with the β-gal. The VEGF injected tissues also contained a visible red blush indicative of leaky vessels. These results show that VEGF can activate angiogenesis/vasculogenesis in non-ischemic tissue.

### Example 12

### Combination Treatments with Other Antiangiogenic Strategies

PDGF Blockade: Herceptin has been approved for the treatment of advanced breast cancer and Gleevec (STI57, imatinib, Novartis Pharma AG) has been approved for chronic myelogenous leukemia and gastrointestinal stromal tumors. Imatinib disrupts the association of pericytes with neovasculature in tumors through effects on PDGFR. While endostatin inhibits early blood vessel formation, imatinib may affect maturation by effects on pericytes. We will initially treat MCF7 and MCF7-HER2 tumors subcutaneously implanted on the left and right flank, respectively, with combination of anti-HER2 IgG3-huEndo fusion proteins and imatinib. Imatinib (50 mg/kg) will be administered orally twice a day. We will examine the blood vessel formation and tumor growth in tumors as outlined supra.

VEGF Blockade:

A humanized anti-VEGF antibody (bevacizumab, Avastin™, rhuMAb-VEGF; Genentech) has been approved for use in combination with chemotherapy in a phase III trial in metastatic colon carcinoma. Avastin has been reported to have clinical benefit of 17% (complete and partial responses plus stable disease 6 months) in phase II trials in breast cancer. Avastin also has activity in renal cell carcinoma, and has been reported to augment taxane activity in a phase III breast cancer trial. Avastin binds and neutralizes all of the major isoforms of VEGF-A, decreases vascular volume, microvascular density, interstitial fluid pressure and the number of viable, circulating endothelial cells. Therefore, combining fusion proteins with Avastin may augment activity of both approaches. We will treat SK-BR-3, or MCF7 and MCF7-HER2 tumors in SCID mice in combination with Avastin (50 μg/injection) and anti-HER2 antibody-huEndo fusion proteins (10, 50, and 250 μg/injection, i.v., q.o.d.) or human endostatin, or antibody alone.

Metronomic Therapy:

Proliferating endothelial cells forming new blood vessels within tumors are sensitive to the cytotoxic effects of many chemotherapeutics. Conventional chemotherapeutic regimes with maximum tolerable doses require extended rest periods which allow repair of the endothelial compartment. However, “metronomic” therapy (i.e. administration of continuous low-doses) may sustain antiangiogenic effects. We will treat MCF7/MCF7-HER2 tumors in SCID mice in combination with various concentrations of anti-HER2 antibody-huEndo fusion proteins (10, 50, and 250 μg/injection, i.v., q.o.d.) and low dose cyclophosphamide (CTX, 25 mg/kg/day, p.o.),79-80 or alone. We will also test repeated administration of low dose taxanes (paclitaxel or docetaxel), using “metronomic” scheduling for the treatment of cancers.

TABLE 1
Pharmacokinetic parameters for [¹²⁵I] labeled proteins in mice with/without tumors

| | | Anti-Dansyl | Anti-HER2/neu | Anti-HER2/neu | |
|---|---|---|---|---|---|
| Mice | Parameter^{a} | IgG3 | IgG3 | IgG3-Endostatin | Endostatin^{b} |
| No Tumor^{c} | T_{1/2}¹ (min): Distribution | | 41.5 ± 26.6 | 28.8 ± 3.4 | 1.69 ± 0.26 |
| | T_{1/2}² (min): Elimination | | 2393 ± 1095 | 2413 ± 174 | 225 ± 116 |
| | AUC₀₋₂₈₈₀(% IDmin/ml) | | 39469 ± 6779 | 23367 ± 5221 | 195 ± 24 |
| | AUC_{0-∞}(% IDmin/ml) | | 88448 ± 35608 | 40033 ± 8119 | 1090 ± 510 |
| | MRT(Min) | | 3285 ± 1450 | 3387 ± 226 | 315 ± 165 |
| CT26 | T_{1/2}¹ (min): Distribution | 157 ± 64 | 96.6 ± 12.3 | 620 ± 508 | 1.65 ± 0.26 |
| | T_{1/2}² (min): Elimination | 2620 ± 131 | 3730 ± 226 | 2600 ± 1360 | 66.2 ± 6.2 |
| | AUC₀₋₅₇₆₀(% IDmin/ml) | 50800 ± 9920 | 69400 ± 3570 | 21500 ± 4540 | 167 ± 10 |
| | AUC_{0-∞}(% IDmin/ml) | 63800 ± 14800 | 103000 ± 5250 | 28200 ± 6540 | 322 ± 38 |
| | MRT(Min) | 3360 ± 373 | 5120 ± 277 | 3960 ± 971 | 86.3 ± 8.6 |
| CT26- | T_{1/2}¹ (min): Distribution | 944 ± 511 | 296 ± 70 | 202 ± 57 | 0.858 ± 0.392 |
| HER2/neu | T_{1/2}² (min): Elimination | 2790 ± 1330 | 3780 ± 403 | 2640 ± 239 | 37.9 ± 17.1 |
| | AUC₀₋₅₇₆₀(% IDmin/ml) | 65000 ± 7370 | 53200 ± 1420 | 11100 ± 1020 | 179 ± 40 |
| | AUC_{0-∞}(% IDmin/ml) | 87200 ± 14500 | 74600 ± 3000 | 13100 ± 1220 | 233 ± 19 |
| | MRT(Min) | 4060 ± 717 | 4580 ± 607 | 2800 ± 63.3 | 49.9 ± 20.9 |
| ^{a}For the pharmacokinetic parameters, the superscript 1 represents the distribution phase and the superscript 2 the elimination phase. AUC₀₋₅₇₆₀ and AUC_{0-∞} are the first 5760 minutes (96 hrs) and steady-state area under the plasma concentration curve respectively. MRT is the mean residence time. To calculate pharmacokinetic parameters, the plasma radioactivity results were fit to a biexponential model (endostatin, anti-HER2/neu IgG3 | | | | | |
| # and anti-HER2/neu IgG3-endostatin) with a derivative-free nonlinear regression analysis. Data are mean ± SEM (n = 3, BALB/c mice). | | | | | |

^{b}Measurements of endostatin was made 60 min after i.v. injection in the mice.
^{c}Measurements of iodine labeled proteins in mice without tumors were made 2880 minutes (48 hrs) after i.v. injection in the mice.

TABLE 2
Specific Tumor Radiolocalization Indices

| | Time | CT26 | CT26-HER2 | Radiolocalization |
|---|---|---|---|---|
| Treatment | (Hrs) | (% ID/g) | (% ID/g) | Indices^{a} |
| Anti-HER2/ | 6 | 3.51 ± 1.38 | 3.94 ± 1.83 | 1.12 |
| neu IgG3 | 24 | 7.04 ± 3.48 | 14.95 ± 3.48 | 2.12 |
| | 96 | 3.03 ± 0.63 | 7.88 ± 2.18 | 2.60 |
| Anti-HER2/ | 6 | 1.16 ± 0.38 | 6.20 ± 0.76 | 5.34 |
| neu IgG3- | 24 | 1.31 ± 0.60 | 9.72 ± 1.05 | 7.42 |
| endostatin | 96 | 0.33 ± 0.05 | 1.17 ± 0.07 | 3.55 |

^{a}Radiolocalization Indices represent the ratios of the % ID/g in CT26-HER2 divided by the % ID/g in CT26.
Data are mean ± SEM (n = 3, BALB/c mice).

TABLE 3
Comparison of the blood vessel density

| | Blood Vessel Area | Blood Vessel Density |
|---|---|---|
| Tumor/Treatment | (pixel²) | (%) |
| CT26/PBS | 19292 ± 5032 | 64 |
| CT26-HER2/PBS | 30242 ± 4317 | 100 |
| CT26/IgG3-Endo | 36326 ± 4361 | 120 |
| CT26-HER2/IgG3-Endo | 4711 ± 736 | 16 |

Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All references cited herein, are incorporated by reference in their entirety.

1. A pharmaceutical composition comprising a chimeric fusion molecule, wherein the chimeric fusion molecule comprises an antigen binding domain and a therapeutic effector domain.

2. The pharmaceutical composition of claim 1, wherein the antigen binding domain comprises an isolated antibody or fragments thereof.

3. The pharmaceutical composition of claim 2, wherein the isolated antibody or fragments thereof comprises immunoglobulin heavy and light chains.

4. The pharmaceutical composition of claim 2, wherein the isolated antibody comprises immunoglobulin variable and constant regions.

5. The pharmaceutical composition of claim 2, wherein the antibody or fragment thereof is any immunoglobulin isotype.

6. The pharmaceutical composition of claim 2, wherein the antibody or fragment thereof, is IgA, IgM, IgG, IgE, or IgD.

7. The pharmaceutical composition of claim 2, wherein the antibody or fragment thereof is IgG1, IgG2, IgG3, and IgG4.

8. The pharmaceutical composition of claim 2, wherein the antibody or fragment thereof is any single chain, two-chain, diabody, minibody, bispecific, multi-chain proteins and glycoproteins belonging to the classes of polyclonal, monoclonal, chimeric, and hetero immunoglobulins.

9. The pharmaceutical composition of claim 2, wherein the antibody or fragment thereof is synthetic and/or genetically engineered variants of any class and isotype immunoglobulins.

10. The pharmaceutical composition of claim 4, wherein the isolated immunoglobulin variable region comprise Fab, Fab′, F(ab′)₂, and Fv fragments.

11. The pharmaceutical composition of claim 4, wherein the isolated immunoglobulin regions comprise immunoglobulin constant regions, C_{H}1, hinge, C_{H}2 and C_{H}3.

12. The pharmaceutical composition of claim 2, wherein the isolated antibody or fragments thereof are fused to a therapeutic effector domain.

13. The pharmaceutical composition of claim 12, wherein the isolated antibody is fused to the therapeutic effector domain via the immunoglobulin constant regions, C_{H}1, hinge, C_{H}2 or C_{H}3.

14. The pharmaceutical composition of claim 13, wherein the isolated antibody is fused to the therapeutic effector domain via the immunoglobulin constant region, C_{H}3.

15. The pharmaceutical composition of claim 1, wherein the therapeutic effector domain comprises a molecule for modulating cellular activity and/or is cytolytic.

16. The pharmaceutical composition of claim 15, wherein the therapeutic effector domain's cellular modulating activity inhibits angiogenesis.

17. The pharmaceutical composition of claim 15, the therapeutic effector domain's cellular modulating activity modulates immune cell responses.

18. The pharmaceutical composition of claim 17, wherein the therapeutic effector domain is selected from the group consisting of endostatin, angioarrestin, angiostatin (plasminogen fragment), anti-angiogenic antithrombin III, cartilage-derived inhibitor (CDI), CD59 complement fragment, fibronectin fragment, gro-beta, heparinases, heparin hexasaccharide fragment, human chorionic gonadotropin (hCG), interferon alpha/beta/gamma, interferon inducible protein (IP-10), interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-b), vasculostatin, and vasostatin (calreticulin fragment).

19. The pharmaceutical composition of claim 18, wherein the therapeutic effector domain is endostatin, angiostatin, basement-membrane collagen-derived anti-angiogenic factors tumstatin, canstatin, or arrestin.

20. The pharmaceutical composition of claim 15, wherein the therapeutic effector domain comprises chemokines, radionuclides and/or interferon.

21. The pharmaceutical composition of claim 20, wherein the nuclides are ⁹⁰Y, ¹³¹I, ¹¹¹In, ¹²⁵I.

22. The pharmaceutical composition of claim 15, wherein the therapeutic effector domain is a cytolytic molecule.

23. The pharmaceutical composition of claim 22, wherein the cytolytic molecule is TNF, enzymes, mediators of apoptosis and/or toxin.

24. The pharmaceutical composition of claim 23, wherein the toxin is selected from the group consisting of as ricin, abrin, diphtheria, gelonin, Pseudomonasexotoxin A, Crotalus durissus terrificus toxin, Crotalus adamenteus toxin, Naja naja toxin, and Naja mocambique toxin.

25. The pharmaceutical composition of claim 23, wherein the mediators of apoptosis include ICE-family of cysteine proteases, apoptin, Bcl-2 family of proteins, Bax, bclXs and caspases.

26. The pharmaceutical composition of claim 23, wherein the enzymes are derived from cytotoxic T lymphocytes or LAK cells.

27. The pharmaceutical composition of claim 26, wherein the enzymes are perform, Fas ligand, and granzymes.

28. The pharmaceutical composition of claim 1, wherein the antibody domain binds to a tumor antigen.

29. The pharmaceutical composition of claim 28, wherein the tumor antigen is HER2/neu or EGFR.

30. An isolated nucleic acid molecule encoding the chimeric molecule of any one of claims 1 through 29.

31. A nucleic acid encoding the chimeric molecule of any one of claims 1 through 29.

32. A chimeric fusion protein comprising a tumor specific antibody or fragment thereof fused to an anti-angiogenic agent.

33. The chimeric fusion protein of claim 32, wherein the tumor specific antibody binds to HER2/neu, EGFR, alpha-actinin-4; BCR-ABL (b3a2); CASP-8; beta-catenin (melanoma); Cdc27; CDK4; dek-can fusion protein; Elongation factor 2; ETV6-AML1 fusion protein; LDLR-fucosyltransferaseAS fusion protein; hsp70-2; KIAA0205; MART2; MUM-If; MUM-2; MUM-3; neo-PAP; Myosin class I; OS-9g; pml-RARalpha fusion protein; PTPRK; K-ras; N-ras; CEA; gp100/Pmel17; Kallikrein 4; mammaglobin-A; Melan-A/MART-1; PSA; TRP-1/gp75; TRP-2; tyrosinase; CPSF; EphA3; G250/MN/CAIX; Intestinal carboxyl esterase; alpha-foetoprotein; M-CSF; MUC1; p53; PRAME; PSMA; RAGE-1; RU2AS; survivin; Telomerase; WT1; and CA125.

34. The chimeric fusion protein of claim 32, wherein the anti-angiogenic agent is endostatin and/or gleevec.

35. The chimeric fusion protein of claim 32, wherein the isolated antibody or fragments thereof comprises immunoglobulin heavy and light chains.

36. The chimeric fusion protein of claim 32, wherein the isolated antibody comprises immunoglobulin variable and constant regions.

37. The chimeric fusion protein of claim 32, wherein the antibody or fragment thereof is any immunoglobulin isotype.

38. The chimeric fusion protein of claim 32, wherein the antibody or fragment thereof, is IgA, IgM, IgG, IgE, or IgD.

39. The chimeric fusion protein of claim 36, wherein the isolated immunoglobulin variable regions comprise Fab, Fab′, F(ab′)₂, and Fv fragments.

40. The chimeric fusion protein of claim 36, wherein the isolated immunoglobulin regions comprise immunoglobulin constant regions, C_{H}1, hinge, C_{H}2 and C_{H}3.

41. The chimeric fusion protein of claim 32, wherein the isolated antibody or fragments thereof are fused to a therapeutic effector domain.

42. The chimeric fusion protein of claim 41, wherein the isolated antibody is fused to the therapeutic effector domain via the immunoglobulin constant regions, C_{H}1, hinge, C_{H}2 or C_{H}3.

43. The chimeric fusion protein of claim 32, wherein the antibody or fragment thereof is IgG1, IgG2, IgG3, and IgG4.

44. The chimeric fusion protein of claim 32, wherein the antibody or fragment thereof is any single chain, two-chain, diabody, minibody, multi-chain proteins and glycoproteins belonging to the classes of polyclonal, monoclonal, chimeric, and hetero immunoglobulins.

45. The chimeric fusion protein of claim 32, wherein the antibody or fragment thereof is synthetic and/or genetically engineered variants of any class and isotype immunoglobulins.

46. The chimeric fusion protein of claim 42, wherein the constant region (C_{H}3) is fused to endostatin, angioarrestin, angiostatin (plasminogen fragment), anti-angiogenic antithrombin III, cartilage-derived inhibitor (CDI), CD59 complement fragment, fibronectin fragment, gro-beta, heparinases, heparin hexasaccharide fragment, human chorionic gonadotropin (hCG), interferon alpha/beta/gamma, interferon inducible protein (IP-10), interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-b), vasculostatin, and vasostatin (calreticulin fragment).

47. The chimeric fusion protein of claim 32, wherein the chimeric fusion protein is administered to a patient in need of such therapy.

48. The chimeric fusion protein of claim 33, wherein the serum half-life of the chimeric fusion protein is at least about 50% greater than the half-life of the anti-HER2/neu antibody.

49. The chimeric fusion protein of claim 33, wherein the serum half-life of the chimeric fusion protein is at least about 80% greater than the half-life of the anti-HER2/neu antibody.

50. The chimeric fusion protein of claim 33, wherein the serum half-life of the chimeric fusion protein is at least about 100% greater than the half-life of the anti-HER2/neu antibody.

51. The chimeric fusion protein of claim 33, wherein the serum half-life of the chimeric fusion protein is at least about 50% greater than the half-life of endostatin.

52. The chimeric fusion protein of claim 33, wherein the serum half-life of the chimeric fusion protein is at least about 80% greater than the half-life of endostatin.

53. The chimeric fusion protein of claim 33, wherein the serum half-life of the chimeric fusion protein is at least about 100% greater than the half-life of endostatin.

54. The chimeric fusion protein of claim 33, wherein the chimeric fusion protein inhibits angiogenesis by at least about 10% as compared to an untreated individual.

55. The chimeric fusion protein of claim 33, wherein the chimeric fusion protein inhibits angiogenesis by at least about 50% as compared to an untreated individual.

56. The chimeric fusion protein of claim 33, wherein the chimeric fusion protein inhibits angiogenesis up to 100% as compared to an untreated individual.

57. A method for targeting endostatin to a tumor cell in an animal subject, the method comprising the step of administering to the animal subject a composition comprising a chimeric molecule comprising an endostatin domain and an Ig domain.

58. A method for treating a tumor in an animal subject, the method comprising the step of administering to the animal subject a chimeric molecule fusion composition, whereby, administration of the composition ameliorates the tumor in the animal subject.

59. The method of claim 58, wherein the antigen binding domain comprises an isolated antibody or fragments thereof.

60. The method of claim 58, wherein the isolated antibody or fragments thereof comprises immunoglobulin heavy and light chains.

61. The method of claim 58, wherein the isolated antibody comprises immunoglobulin variable and constant regions.

62. The method of claim 58, wherein the antibody or fragment thereof is any immunoglobulin isotype.

63. The method of claim 58, wherein the antibody or fragment thereof, is IgA, IgM, IgG, IgE, or IgD.

64. The method of claim 58, wherein the antibody or fragment thereof is IgG1, IgG2, IgG3, and IgG4.

65. The method of claim 58, wherein the antibody or fragment thereof is any single chain, two-chain, diabody, minibody, bispecific, multi-chain proteins and glycoproteins belonging to the classes of polyclonal, monoclonal, chimeric, and hetero immunoglobulins.

66. The method of claim 58, wherein the antibody or fragment thereof is synthetic and/or genetically engineered variants of any class and isotype immunoglobulins.

67. The method of claim 61, wherein the isolated immunoglobulin variable region comprise Fab, Fab′, F(ab′)₂, and Fv fragments.

68. The method of claim 61, wherein the isolated immunoglobulin regions comprise immunoglobulin constant regions, C_{H}1, hinge, C_{H}2 and C_{H}3.

69. The method of claim 68, wherein the isolated antibody or fragments thereof are fused to a therapeutic effector domain.

70. The method of claim 69, wherein the isolated antibody is fused to the therapeutic effector domain via the immunoglobulin constant regions, C_{H}1, hinge, C_{H}2 or C_{H}3.

71. The method of claim 70, wherein the isolated antibody is fused to the therapeutic effector domain via the immunoglobulin constant region, C_{H}3.

72. The method of claim 58, wherein the therapeutic effector domain comprises a molecule for modulating cellular activity or is cytolytic.

73. The method of claim 72, wherein the therapeutic effector domain's cellular modulating activity inhibits angiogenesis.

74. The method of claim 72, the therapeutic effector domain's cellular modulating activity modulates immune cell responses.

75. The method of claim 72, wherein the therapeutic effector domain is selected from the group consisting of endostatin, angioarrestin, angiostatin (plasminogen fragment), anti-angiogenic antithrombin III, cartilage-derived inhibitor (CDI), CD59 complement fragment, fibronectin fragment, gro-beta, heparinases, heparin hexasaccharide fragment, human chorionic gonadotropin (hCG), interferon alpha/beta/gamma, interferon inducible protein (IP-10), interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-b), vasculostatin, and vasostatin (calreticulin fragment).

76. The method of claim 72, wherein the therapeutic effector domain is endostatin, angiostatin, basement-membrane collagen-derived anti-angiogenic factors tumstatin, canstatin, or arrestin.

77. The method of claim 72, wherein the therapeutic effector domain comprises chemokines, radionuclides and/or interferon.

78. The method of claim 77, wherein the nuclides are ⁹⁰Y, ¹³¹I, ¹¹¹In, ¹²⁵I.

79. The method of claim 72, wherein the therapeutic effector domain is a cytolytic molecule.

80. The method of claim 79, wherein the cytolytic molecule is TNF, enzymes, mediators of apoptosis and/or toxin.

81. The method of claim 80, wherein the toxin is selected from the group consisting of as ricin, abrin, diphtheria, gelonin, Pseudomonasexotoxin A, Crotalus durissus terrificus toxin, Crotalus adamenteus toxin, Naja naja toxin, and Naja mocambique toxin.

82. The method of claim 80, wherein the mediators of apoptosis include ICE-family of cysteine proteases, apoptin, Bcl-2 family of proteins, Bax, bclXs and caspases.

83. The method of claim 80, wherein the enzymes are derived from cytotoxic T lymphocytes or LAK cells.

84. T The method of claim 80, wherein the enzymes are perforin, Fas ligand, and granzymes.

85. The method of claim 58, wherein the antibody domain binds to a tumor antigen.

86. The method of claim 85, wherein the tumor antigen is HER2/neu or EGFR.

87. The method of claim 58, wherein the chimeric fusion molecule composition is administered with one or more therapeutic agents and/or adjuvants.

88. The method of claim 87, wherein the therapeutic agents comprise antiangiogenic antibodies, tumor antigen specific antibodies, glycolysis inhibitor agents, anti-angiogenic agents, chemotherapeutic agents, radiotherapy, radionuclides, or drugs that ameliorate the symptoms of a patient.

89. The method of claim 58, wherein the chimeric fusion molecule composition is administered to a patient in combination with metronomic therapy.

90. A kit comprising:
a chimeric molecule comprising a domain targeting the chimeric molecule to HER2/neu tumor antigen and a domain comprising an anti-angiogenic agent.

91. The kit of claim 90, wherein the domain comprising the anti-angiogenic agent is endostatin of fragments thereof.

92. The kit of claim 90, wherein the domain targeting the chimeric molecule to HER2/neu tumor antigen is an antibody or fragments thereof.

93. The kit of claim 91, wherein the antibody or fragments thereof is polyclonal or monoclonal.

94. The kit of claim 90, wherein the kit further comprises a pharmaceutical composition.

95. The kit of claim 90, wherein instructions for carrying out the method are provided.


SEQUENCE LISTING (6 sequences)

SEQ ID NO: 1 - DNA, 36 bp, Mus musculus
cccctcgcgatatcatactcatcaggactttcagcc

SEQ ID NO: 2 - DNA, 40 bp, Mus musculus
ccccgaattcgttaacctttggagaaagaggtcatgaagc

SEQ ID NO: 3 - DNA, 18 bp, Homo sapiens
atggcagaagggcagcat

SEQ ID NO: 4 - DNA, 24 bp, Homo sapiens
ttggtgaggtttgatccgcatcat

SEQ ID NO: 5 - DNA, 21 bp, Homo sapiens
ccatgaactttctgctgtctt

SEQ ID NO: 6 - DNA, 21 bp, Homo sapiens
tcgatcgttctgtatcagtct